SEQUENCE LISTING (46 sequences)

SEQ ID NO: 1 - protein, 30 aa, synthetic construct
  REGION 1..30: note = Synthetic
QKGTEEFVKILRYENEVLQLEEDERFEGRV

SEQ ID NO: 2 - protein, 20 aa, synthetic construct
  REGION 1..20: note = Synthetic
FVKILRYENEVLQLEEDERF

SEQ ID NO: 3 - protein, 39 aa, synthetic construct
  REGION 1..39: note = Synthetic
FVKILRYENEVLQLEEDERGFVKILRYENEVLQLEEDER

SEQ ID NO: 4 - protein, 6 aa, synthetic construct
  REGION 1..6: note = Synthetic
LQLEED

SEQ ID NO: 5 - protein, 19 aa, synthetic construct
  REGION 1..19: note = Synthetic
LQLEEDERFGLQLEEDERF

SEQ ID NO: 6 - protein, 21 aa, synthetic construct
  REGION 1..21: note = Synthetic
CLQLEEDERFGLQLEEDERFC

SEQ ID NO: 7 - protein, 7 aa, synthetic construct
  REGION 1..7: note = Synthetic
LEEDERF

SEQ ID NO: 8 - protein, 7 aa, synthetic construct
  REGION 1..7: note = Synthetic
KILRYEN

SEQ ID NO: 9 - protein, 7 aa, synthetic construct
  REGION 1..7: note = Synthetic
YENEVLQ

SEQ ID NO: 10 - protein, 9 aa, synthetic construct
  REGION 1..9: note = Synthetic
LQLEEDERF

SEQ ID NO: 11 - protein, 218 aa, Homo sapiens
MGRLLALVVGAALVSSACGGCVEVDSETEAVYGMTFKILCISCKRRSETNAETFTEWTFRQKGTEEFVKILRYENEVLQLEEDERFEGRVVWNGSRGTKDLQDLSIFITNVTYNHSGDYECHVYRLLFFENYEHNTSVVKKIHIEVVDKANRDMASIVSEIMMYVLIVVLTIWLVAEMIYCYKKIAAATETAAQENASEYLAITSESKENCTGVQVAE

SEQ ID NO: 12 - protein, 268 aa, Homo sapiens
MGRLLALVVGAALVSSACGGCVEVDSETEAVYGMTFKILCISCKRRSETNAETFTEWTFRQKGTEEFVKILRYENEVLQLEEDERFEGRVVWNGSRGTKDLQDLSIFITNVTYNHSGDYECHVYRLLFFENYEHNTSVVKKIHIEVVDKGESGAACPFTVTHRRARWRDRWQAVDRTGWLCAWPANRPQQRAEGEGSSPSCPLQLWPLFLSSPRRGQSMPVPHRRSGYRTQLCHLCCMTSGRCLLSLSQRVVLGLPGIIIRCVSRGVV

SEQ ID NO: 13 - protein, 20 aa, synthetic construct
  REGION 1..20: note = Synthetic
FEELELLKVVNFQRDEIYER

SEQ ID NO: 14 - protein, 20 aa, synthetic construct
  REGION 1..20: note = Synthetic
FVKILRYENEVLQLEEDEDF

SEQ ID NO: 15-28 - skipped (14 entries)

SEQ ID NO: 29 - protein, 10 aa, synthetic construct
  REGION 1..10: note = Synthetic
  SITE 2..4: note = X is any amino acid
  SITE 6..7: note = X is any amino acid
  SITE 9: note = X is any amino acid
LXXXLXXLXL

SEQ ID NO: 30 - protein, 4 aa, synthetic construct
  REGION 1..4: note = Synthetic
KDEL

SEQ ID NO: 31-36 - skipped (6 entries)

SEQ ID NO: 37 - protein, 215 aa, Homo sapiens
MHRDAWLPRPAFSLTGLSLFFSLVPPGRSMEVTVPATLNVLNGSDARLPCTFNSCYTVNHKQFSLNWTYQECNNCSEEMFLQFRMKIINLKLERFQDRVEFSGNPSKYDVSVMLRNVQPEDEGIYNCYIMNPPDRHRGHGKIHLQVLMEEPPERDSTVAVIVGASVGGFLAVVILVLMVVKCVRRKKEQKLSTDDLKTEEEGKTDGEGNPDDGAK

SEQ ID NO: 38 - protein, 24 aa, Homo sapiens
WTYQECNNCSEEMFLQFRMKIINL

SEQ ID NO: 39 - protein, 215 aa, Homo sapiens
MPAFNRLFPLASLVLIYWVSVCFPVCVEVPSETEAVQGNPMKLRCISCMKREEVEATTVVEWFYRPEGGKDFLIYEYRNGHQEVESPFQGRLQWNGSKDLQDVSITVLNVTLNDSGLYTCNVSREFEFEAHRPFVKTTRLIPLRVTEEAGEDFTSVVSEIMMYILLVFLTLWLLIEMIYCYRKVSKAEEAAQENASDYLAIPSENKENSAVPVEE

SEQ ID NO: 40 - protein, 32 aa, Homo sapiens
WFYRPEGGKDFLIYEYRNGHQEVESPFQGRLQ

SEQ ID NO: 41 - protein, 228 aa, Homo sapiens
MPGAGDGGKAPARWLGTGLLGLFLLPVTLSLEVSVGKATDIYAVNGTEILLPCTFSSCFGFEDLHFRWTYNSSDAFKILIEGTVKNEKSDPKVTLKDDDRITLVGSTKEKMNNISIVLRDLEFSDTGKYTCHVKNPKENNLQHHATIFLQVVDRLEEVDNTVTLIILAVVGGVIGLLILILLIKKLIIFILKKTREKKKECLVSSSGNDNTENGLPGSKAEEKPPSKV

SEQ ID NO: 42 - protein, 35 aa, Homo sapiens
WTYNSSDAFKILIEGTVKNEKSDPKVTLKDDDRIT

SEQ ID NO: 43 - protein, 7 aa, synthetic construct
  REGION 1..7: note = Synthetic
  SITE 1: note = X is S or T
  SITE 4: note = X is any amino acid
XRRXFLK

SEQ ID NO: 44 - protein, 15 aa, synthetic construct
  REGION 1..15: note = Synthetic
GGGGGGGGGGGGGGG

SEQ ID NO: 45 - protein, 15 aa, synthetic construct
  REGION 1..15: note = Synthetic
GGGGSGGGGSGGGGS

SEQ ID NO: 46 - protein, 8 aa, synthetic construct
  REGION 1..8: note = Synthetic
GPLGIAGQ

(19) United States
(12) Patent Application Publication
(10) Pub. No.: US 2025/0304649 A1
(43) Pub. Date: Oct. 2, 2025

(54) SCN1B MIMETIC PEPTIDES AND USES THEREOF

(71) Applicant: Virginia Tech Intellectual Properties, Inc., Blacksburg, VA (US)

(72) Inventors: Zachary Williams, Blacksburg, VA (US); Linda Jane Jourdan, Blacksburg, VA (US); Daniel Hoagland, Blacksburg, VA (US); Steven Poelzing, Blacksburg, VA (US); Robert G. Gourdie, Blacksburg, VA (US)

(21) Appl. No.: 18/862,277

(22) PCT Filed: Mar. 1, 2023
(86) PCT No.: PCT/US2023/063525

§ 371 Date: Nov. 1, 2024

Related U.S. Application Data
(60) Provisional application No. 63/337,408, filed on May 2, 2022.

(51) Int. Cl. C07K 14/705; A61K 38/00

(52) U.S. Cl. CPC C07K 14/705; A61K 38/00

(57) ABSTRACT

Described in certain example embodiments herein are voltage gated sodium channel beta subunit 1 mimetic polypeptides, polynucleotides encoding the same, and formulations thereof. Described in certain example embodiments, are methods of inhibiting or otherwise modulating voltage gated sodium channel activity by administering the voltage gated sodium channel beta subunit 1 mimetic polypeptides, polynucleotides encoding the same, and/or formulations thereof to a subject in need thereof.

### CROSS-REFERENCE TO RELATED APPLICATIONS

This application claims the benefit of and priority to co-pending U.S. Provisional Patent Application No. 63/337,408, filed on May 2, 2022, entitled “SCN1B MIMETIC PEPTIDES AND USES THEREOF,” the contents of which is incorporated by reference herein in its entirety.

### STATEMENT REGARDING FEDERALLY SPONSORED RESEARCH

This invention was made with government support under Grant Nos. HL161237, HL141855, R01HL056728, and 5R01HL141855 awarded by the National Institutes of Health. The government has certain rights in the invention.

### SEQUENCE LISTING

This application contains a sequence listing filed in electronic form as an .xml file entitled VTIP-0370WP_ST26.xml, created on Mar. 1, 2023 and having a size of 37,011 bytes. The content of the sequence listing is incorporated herein in its entirety.

### TECHNICAL FIELD

The subject matter disclosed herein is generally directed to voltage-gate sodium channel subunit protein therapeutics and uses thereof.

### BACKGROUND

Voltage-gated sodium channels (VGSCs) are responsible for initiation and propagation of action potentials in excitable cells. Mutations in the genes encoding β subunits are linked to a number of diseases, including epilepsy, sudden death syndromes like SUDEP and SIDS, and cardiac arrhythmia. Currently there are no β subunit-specific therapeutics available. As such, there exists a need for therapeutics directed to voltage-gated sodium channels and associated proteins, and more particularly the β subunit.

Citation or identification of any document in this application is not an admission that such a document is available as prior art to the present invention.

### SUMMARY

Described in certain example embodiments herein are engineered polypeptides comprising or consisting of one or more βadp1 mimetic peptides (also referred to herein as SCN1B mimetic peptides) or one or more functional domains thereof, one or more derivatives thereof, and/or one or more homologues thereof.

In certain example embodiments, the one or more βadp1 mimetic peptides or one or more functional domains thereof are selected from SEQ ID NOs: 2-10, 38, 40, or 42.

In certain example embodiments, the one or more βadp1 mimetic peptides or one or more functional domains thereof do not comprise or consist of a monomeric peptide having a sequence identical to SEQ ID NO: 2.

In certain example embodiments, the engineered polypeptide comprises a homodimer dimer comprising two monomers of any one of SEQ ID NOs.: 2, 4, 7, 8, 9, 10, 38, 40, and 42.

Described in certain example embodiments herein are polynucleotides that encode any of the engineered polypeptides of any one of the preceding paragraphs and described elsewhere herein.

In certain example embodiments, the polynucleotide is DNA or RNA.

Described in certain example embodiments herein are vector or vector systems comprising a polynucleotide of any one of the preceding paragraphs and as described elsewhere herein that encodes any of the engineered polypeptides of any one of the preceding paragraphs and as described elsewhere herein.

Described in certain example embodiments herein are delivery vehicles comprising one or more engineered polypeptides of any one of the preceding paragraphs and as described elsewhere herein, the polynucleotide of any one of the preceding paragraphs and as described elsewhere herein, the vector or vector system any one of the preceding paragraphs and as described elsewhere herein, or any combination thereof.

In certain example embodiments, the delivery vehicle is a liposome, a micelle, or an exosome.

Described in certain example embodiments herein is a cell or cell population comprising and/or capable of producing one or more engineered polypeptides any one of the preceding paragraphs and as described elsewhere herein, the polynucleotide any one of the preceding paragraphs and as described elsewhere herein, the vector or vector system any one of the preceding paragraphs and as described elsewhere herein, a delivery vehicle of any one any one of the preceding paragraphs and as described elsewhere herein, or any combination thereof.

Described in certain example embodiments herein are pharmaceutical formulation comprising one or more engineered polypeptides any one of the preceding paragraphs and as described elsewhere herein, the polynucleotide any one of the preceding paragraphs and as described elsewhere herein, the vector or vector system any one of the preceding paragraphs and as described elsewhere herein, the delivery vehicle any one of the preceding paragraphs and as described elsewhere herein, the cell or cell population any one of the preceding paragraphs and as described elsewhere herein, or any combination thereof; and a pharmaceutically acceptable carrier.

Described in certain example embodiments herein are methods of treating a SCN1B disease, condition, or disorder in a subject in need thereof, the method comprising: administering, to the subject in need thereof, one or more engineered polypeptides any one of the preceding paragraphs and as described elsewhere herein, the polynucleotide any one of the preceding paragraphs and as described elsewhere herein, the vector or vector system any one of the preceding paragraphs and as described elsewhere herein, the delivery vehicle any one of the preceding paragraphs and as described elsewhere herein, the cell or cell population any one of the preceding paragraphs and as described elsewhere herein, a pharmaceutical formulation any one of the preceding paragraphs and as described elsewhere herein, or any combination thereof.

In certain example embodiments, administering occurs for a time period of up to 2 hours.

In certain example embodiments, administering occurs for a time period of 2 hours or more, optionally for 2, 4, 6, 8, 12, 24, 48, or more hours.

In certain example embodiments, the SCN1B mediated disease, condition, or disorder is a cardiac arrythmia and/or other cardiac contractility/fibrillation disorder and/or dysfunction, an epilepsy, a neurodegenerative disease (including, but not limited to, Alzheimer's Disease, dementias, and/or the like), a neuropathy, pain, cancer (including but not limited to cervical cancer, breast cancer, and prostate cancer), an Autism spectrum disorder, a mood disorder, or any combination thereof.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1 (SEQ ID NO: 1-9, 13-14) shows a table of SCN1B and exemplary mimetic peptides.

FIG. 2 shows a schematic of regulated intramembrane proteolysis of VGSC β1 subunit that shows the sequential cleavage steps of the β1 subunit. Full length β1 is 37 kD, while the first cleavage product, the CTF, is 19 kD. The ICD is the final cleavage product and translocates to the nucleus, resulting in transcriptional changes. The dashed arrow portrays a hypothetical effect of βadp1 treatment.

FIGS. 3A-3E show βadp1 binding and identification of βadp1 derived smaller peptide fragments with similar function. FIG. 3A shows a homology model of the β1/SCN1B extracellular domain based on β3 crystal structure (3), with βadp1 (stick model) docked in silico in a low-energy conformation with the adhesion surface of the β1 immunogloublin (Ig) loop. FIG. 3B shows a rendering of βadp1 (ball and stick model) docked to the predicted Ig adhesion surface of a β1 homology model. This perspective of the adhesion surface tension reveals a hydrophobic pocket, with which hydrophobic amino acids towards the C-terminus of βadp1 e.g., LQL) are predicted to interact with β1. Positive and negatively charged moieties on the adhesion surface of the β1 Ig loop are shown in red and blue (as represented in greyscale), respectively. FIG. 3C (SEQ ID NO: 4) shows a rendering of βadp1 (ball and stick model)-based sequence LQLEED (SEQ ID NO: 4) docked with β1, showing a predicted interaction like that of βadp1 between the LQL motif and the hydrophobic pocket on the adhesion surface of the β1 Ig domain. FIG. 3D (SEQ ID NO: 2, 4, 7-9) shows results of a multi-well ECIS demonstrating activity of βadp1, and short 6-8 amino acid N-terminal truncation variant peptides of βadp1, in reducing normalized resistance of monolayers in 1610 cells exogenously expressing β1/SCN1B (a marker of levels of cell adhesion mediated by β1/SCN1B) over 20 hours. As previously reported, βadp1 significantly reduces normalized resistance in these cells relative to control over this time course. Peptides towards the carboxyl terminus of βadp1, especially those incorporating hydrophobic leucine residues, show the most adhesion reducing activity. Based on the ECIS time course data, and predictions from molecular modeling of likely binding in the hydrophobic pocket β1/SCN1B Ig domain binding surface, the sequence LQLEED (SEQ ID NO: 4) was selected as a short peptide candidate drug molecule. FIG. 3E (SEQ ID NO: 7-9) shows an ECIS comparison of effects on normalized resistance in monolayers of 1610 cells exogenously expressing β1/SCN1B at 20 hours following treatment with 10 μM and 100 μM βadp1, various c-terminal fragments of βadp1, and vehicle control.

FIG. 4A-4B show that Db1-βadp1 increase resistance in 1610β1 cells over 24 hours of treatment. (FIG. 4A) Multi-well ECIS demonstrating activity of Db1-βadp1 at different concentrations of 100 μM and 50 μM compared to DMSO. FIG. 4B shows Quantification of FIG. 4A. Data has n=2 so no statistical significance can be calculated but both figures show a concentration dependent effect of Db1-βadp1 on increasing resistance.

FIGS. 5A-5E demonstrate that βadp1 and LQLEED (SEQ ID NO: 4) decrease resistance in 1610β1 cells while βS2C and βS2L increase resistance. (FIG. 5A) Multi-well ECIS demonstrating activity of βadp1 derived dimers, βS2C and βS2L compared to DMSO vehicle treated cells. Both βS2C and βS2L increase resistance compared to DMSO vehicle at 5 and 20 hours of a single treatment given at the time of plating the cells. At 40 hours βS2L continues to increase resistance while βS2C is trending towards an increase. (FIG. 5B) (SEQ ID NO: 4) Multi-well ECIS demonstrating activity of βadp1 and βadp1 derived peptide LQLEED (SEQ ID NO: 4) compared to DMSO vehicle treated cells over a longer time period than has previously been shown. Both βadp1 and LQLEED (SEQ ID NO: 4) decrease resistance compared to DMSO at 5 and 20 hours of treatment. There is a shift in the curve of βadp1 at approximately 30 hours showing increased resistance beyond this timepoint (arrow). As a result, at 40 hours there is no significant difference between βadp1 or LQLEED (SEQ ID NO: 4) compared to DMSO, suggesting a change in functional outcome of these two peptides after the 30 hour timepoint. (FIGS. 5C-5E) (SEQ ID NO: 4) Quantification of ECIS data at 5, 20, and 40 hours after treatment.

FIG. 6 shows βadp1 treatment over time results in greater β1 abundance. 1610β1 cells were plated and treated simultaneously with 50 μM Biotin-βadp1 or 0.1% DMSO on 18 mm round glass coverslips. Cells were then allowed to grow for 1-48 hours, after which they were fixed with 4% paraformaldehyde. Biotin-βadp1 was labeled with Streptavidin-Alexa647 conjugate and SCN1B labeled with Alexa 568. The top row shows an increase of Biotin-βadp1 up to 24 hours, where the maximum signal occurs mainly intracellularly. By 48 hours, the signal has largely been decreased. The middle row shows the effect of the Biotin-βadp1 treatment on β1 abundance in the 1610β1 cells. There is a steady increase over time, with the greatest effect seen at 48 hours. The bottom row shows the effect of the DMSO control treatment, with no change in β1 abundance over time. White scale bar: 10 μm

FIGS. 7A-7E demonstrate that each βadp1 derived peptide increases β1 abundance at 48 hours of treatment. FIGS. 7A-7D show 1610β1 cells treated for 48 hours with 10 μM concentrations of DMSO or βadp1 derived peptides (FIG. 7A) 0.1% DMSO, (FIG. 7B) βadp1, (FIG. 7C) LQLEED (SEQ ID NO: 4), (FIG. 7D) βS2L. Each peptide shows an increase in β1 abundance compared to DMSO treated cells. (FIG. 7E) Closeup view of indicated portion of βS2L treated cells. βS2L increased the overall β1 abundance in 1610β1 cells, but unlike the other βadp1 derived peptides, it also increase the 1 abundance at cell-cell borders (white arrows), which is an indication of how βS2L could be increasing resistance over time in ECIS experiments.

FIGS. 8A-8C show that βadp1 increases the amount of regulated intramembrane (RIP) proteolysis the β1 subunit undergoes. (FIG. 8A) Representative Western blot showing 0.1% DMSO, 50 μM βadp1, 1 μM DAPT, and 50 μM+1 μM DAPT treatments at 6 hours, 24 hours, and 48 hours of treatment. Cells were plated and treated simultaneously, and then lysate was collected at the indicated 6, 24, or 48 hours. Detection of the β1 subunit was done using a c-terminal antibody “Na Channel β1 Subunit (D4Z2N) Rabbit mAb #13950” (Cell Signaling Technologies). This allowed for labeling of both the full length β1 protein (37 kD) and the C-terminal fragment (CTF) (19 kD). Cells were treated with DAPT, an inhibitor of y-secretase which is responsible for the second cleavage step of the RIP of β1, resulting in an accumulation of the CTF. The blots show that the CTF is typically undetectable at normal exposure times unless cells are treated with DAPT. When cells are co-treated with DAPT and βadp1, there is a greater accumulation of the CTF compared to DAPT treatment alone, indicating that βadp1 is increasing the amount of RIP that β1 undergoes. (FIG. 8B) Quantification of the 37 kD full-length β1 protein at 6, 24, and 48 hours. All results are normalized to DMSO treatment at each timepoint and then shown as a ratio of DAPT+βadp1/DAPT to account for the DAPT treatment in each. There is a significant increase in full-length β1 6 hours after co-treatment, which then decreases across the 24 and 48 hour timepoints. (FIG. 8C) Quantification of the 19 kD CTF of β1. All results are normalized to DMSO treatment at each timepoint and then shown as a ratio of DAPT+βadp1/DAPT to account for the DAPT treatment in each. There is a significant increase in the CTF after co-treatment compared to DAPT across all timepoints, indicating the increased cleavage of full-length β1 into the CTF.

FIG. 9A-9H demonstrate that βadp1 and LQLEED (SEQ ID NO: 4) decrease resistance in 1610β1 cells while βS2C and βS2L increase resistance. (FIG. 9A) Multi-well ECIS demonstrating activity of βadp1 derived dimers, βS2C and βS2L compared to DMSO vehicle treated cells. Both βS2C and βS2L increase resistance compared to DMSO vehicle at 5 and 20 hours of a single treatment given at the time of plating the cells. At 40 hours βS2L continues to increase resistance while βS2C is trending towards an increase. (FIG. 9B) (SEQ ID NO: 4) Multi-well ECIS demonstrating activity of βadp1 and βadp1 derived peptide LQLEED (SEQ ID NO: 4) compared to DMSO vehicle treated cells over a longer time period than has previously been shown. Both βadp1 and LQLEED (SEQ ID NO: 4) decrease resistance compared to DMSO at 5 and 20 hours of treatment. There is a shift in the curve of βadp1 at approximately 30 hours showing increased resistance beyond this timepoint (arrow). As a result, at 40 hours there is no significant difference between βadp1 or LQLEED (SEQ ID NO: 4) compared to DMSO, suggesting a change in functional outcome of these two peptides after the 30 hour timepoint. FIG. 9C-9H show the effect of treatment with the inhibitor (FIG. 9C, 9E, 9G) as compared to the dimer (FIG. 9D, 9F, 9H) at 5, 20, and 40 hours post treatment with the inhibitor or dimer.

FIG. 10 shows βadp1 treatment over time results in greater β1 abundance. 1610β1 cells were plated and treated simultaneously with 50 μM Biotin-βadp1 or 0.1% DMSO on 18 mm round glass coverslips. Cells were then allowed to grow for 0.5-48 hours, after which they were fixed with 4% paraformaldehyde. Biotin-βadp1 was labeled with Streptavidin-Alexa647 conjugate and SCN1B labeled with Alexa 568. The top row shows an increase of Biotin-βadp1 up to 24 hours, where the maximum signal occurs mainly intracellularly. By 48 hours, the signal has largely been decreased. The middle row shows the effect of the Biotin-βadp1 treatment on β1 abundance in the 1610β1 cells. There is a steady increase over time, with the greatest effect seen at 48 hours. The bottom row shows the effect of the DMSO control treatment, with no change in β1 abundance over time. White scale bar: 10 μm

FIG. 11-shows a schematic of regulated intramembrane proteolysis of VGSC β1 subunit that shows the sequential cleavage steps of the β1 subunit. Full length β1 is 37 kD, while the first cleavage product, the CTF, is 19 kD. The ICD is the final cleavage product and translocates to the nucleus, resulting in transcriptional changes. The dashed arrow portrays a hypothetical effect of βadp1 treatment.

FIG. 12A-12C show results from cells treated with the γ-secretase inhibitor DAPT and cells co-treated with DAPT in the presence of βadp1. FIG. 12A shows Western blots from cells sampled at 6, 24 and 48 hours following initiation of treatment. FIG. 12B-12C shows quantification of immunoblots sampled at 6, 24 and 48 hours following initiation of treatment.

FIG. 13A-13C show results from cells treated with the γ-secretase inhibitor DAPT and cells co-treated with DAPT in the presence of PSL2. FIG. 12A shows Western blots from cells sampled at 6, 24 and 48 hours following initiation of treatment. FIG. 12B-12C shows quantification of immunoblots sampled at 6, 24 and 48 hours following initiation of treatment.

FIG. 14A-14H show immunoblot results from cells treated with the γ-secretase inhibitor DAPT, PSL2, βadp1, or R85D, or co-treated with DAPT and PSL2 (the dimer), βadp1, or R85D (FIG. 14A-14B) and the effect of the full length or truncated 1 (FIG. 14C-14H).

FIG. 15A-15D show results from inhibition of the RIP of $1.

The figures herein are for illustrative purposes only and are not necessarily drawn to scale.

### DETAILED DESCRIPTION OF THE EXAMPLE EMBODIMENTS

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Where a range is expressed, a further aspect includes from the one particular value and/or to the other particular value. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and subrange is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes I X, published by Jones and Bartlett, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2^{nd} ed., J. Wiley & Sons (New York, N.Y. 1994), March, Advanced Organic Chemistry Reactions, Mechanisms and Structure 4^{th} ed., John Wiley & Sons (New York, N.Y. 1992); and Marten H. Hofker and Jan van Deursen, Transgenic Mouse Methods and Protocols, 2^{nd} edition (2011).

Definitions of common terms and techniques in chemistry and organic chemistry can be found in Smith. Organic Synthesis, published by Academic Press. 2016; Tinoco et al. Physical Chemistry, 5^{th} edition (2013) published by Pearson; Brown et al., Chemistry, The Central Science 14^{th} ed. (2017), published by Pearson, Clayden et al., Organic Chemistry, 2^{nd} ed. 2012, published by Oxford University Press; Carey and Sunberg, Advanced Organic Chemistry, Part A: Structure and Mechanisms, 5^{th} ed. 2008, published by Springer; Carey and Sunberg, Advanced Organic Chemistry, Part B: Reactions and Synthesis, 5^{th} ed. 2010, published by Springer, and Vollhardt and Schore, Organic Chemistry, Structure and Function; 8^{th} ed. (2018) published by W. H. Freeman.

As used herein, the singular forms “a”, “an”, and “the” include both singular and plural referents unless the context clearly dictates otherwise.

As used herein, “about,” “approximately,” “substantially,” and the like, when used in connection with a measurable variable such as a parameter, an amount, a temporal duration, and the like, are meant to encompass variations of and from the specified value including those within experimental error (which can be determined by e.g. given data set, art accepted standard, and/or with e.g. a given confidence interval (e.g. 90%, 95%, or more confidence interval from the mean), such as variations of +/−10% or less, +/−5% or less, +/−1% or less, and +/−0.1% or less of and from the specified value, insofar such variations are appropriate to perform in the disclosed invention. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

As used herein, a “biological sample” refers to a sample obtained from, made by, secreted by, excreted by, or otherwise containing part of or from a biologic entity. A biologic sample can contain whole cells and/or live cells and/or cell debris, and/or cell products, and/or virus particles. The biological sample can contain (or be derived from) a “bodily fluid”. The biological sample can be obtained from an environment (e.g., water source, soil, air, and the like). Such samples are also referred to herein as environmental samples. As used herein “bodily fluid” refers to any non-solid excretion, secretion, or other fluid present in an organism and includes, without limitation unless otherwise specified or is apparent from the description herein, amniotic fluid, aqueous humor, vitreous humor, bile, blood or component thereof (e.g., plasma, serum, etc.), breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from an organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

As used herein, “administering” refers to any suitable administration for the agent(s) being delivered and/or subject receiving said agent(s) and can be oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, by inhalation, by catheters, stents or via an implanted reservoir or other device that administers, either actively or passively (e.g., by diffusion) a composition the perivascular space and adventitia. For example, a medical device such as a stent can contain a composition or formulation disposed on its surface, which can then dissolve or be otherwise distributed to the surrounding tissue and cells. The term “parenteral” can include subcutaneous, intravenous, intramuscular, intra-synovial, intrasternal, intrathecal, intrahepatic, intralesional, and intracranial injections or infusion techniques. Administration routes can be, for instance, auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal (dental), intracoronary, intracorporus cavernosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratym panic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, subarachnoid, subconjunctival, subcutaneous, sublingual, submucosal, topical, transdermal, transmucosal, transplacental, transtracheal, transtympanic, ureteral, urethral, and/or vaginal administration, and/or any combination of the above administration routes, which typically depends on the disease to be treated, subject being treated, and/or agent(s) being administered.

As used herein, “agent” refers to any substance, compound, molecule, and the like, which can be administered to a subject on a subject to which it is administered to. An agent can be inert. An agent can be an active agent. An agent can be a primary active agent, or in other words, the component(s) of a composition to which the whole or part of the effect of the composition is attributed. An agent can be a secondary agent, or in other words, the component(s) of a composition to which an additional part and/or other effect of the composition is attributed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment but may. Furthermore, the particular features, structures or characteristics may be combined in any suitable manner, as would be apparent to a person skilled in the art from this disclosure, in one or more embodiments. Furthermore, while some embodiments described herein include some, but not other features included in other embodiments, combinations of features of different embodiments are meant to be within the scope of the invention. For example, in the appended claims, any of the claimed embodiments can be used in any combination.

As used herein “cancer” refers to one or more types of cancer including, but not limited to, acute lymphoblastic leukemia, acute myeloid leukemia, adrenocortical carcinoma, Kaposi Sarcoma, AIDS-related lymphoma, primary central nervous system (CNS) lymphoma, anal cancer, appendix cancer, astrocytomas, atypical teratoid/Rhabdoid tumors, basal cell carcinoma of the skin, bile duct cancer, bladder cancer, bone cancer (including but not limited to Ewing Sarcoma, osteosarcomas, and malignant fibrous histiocytoma), brain tumors, breast cancer, bronchial tumors, Burkitt lymphoma, carcinoid tumor, cardiac tumors, germ cell tumors, embryonal tumors, cervical cancer, cholangiocarcinoma, chordoma, chronic lymphocytic leukemia, chronic myelogenous leukemia, chronic myeloproliferative neoplasms, colorectal cancer, craniopharyngioma, cutaneous T-Cell lymphoma, ductal carcinoma in situ, endometrial cancer, ependymoma, esophageal cancer, esthesioneuroblastoma, extracranial germ cell tumor, extragonadal germ cell tumor, eye cancer (including, but not limited to, intraocular melanoma and retinoblastoma), fallopian tube cancer, gallbladder cancer, gastric cancer, gastrointestinal carcinoid tumor, gastrointestinal stromal tumors, central nervous system germ cell tumors, extracranial germ cell tumors, extragonadal germ cell tumors, ovarian germ cell tumors, testicular cancer, gestational trophoblastic disease, Hairy cell leukemia, head and neck cancers, hepatocellular (liver) cancer, Langerhans cell histiocytosis, Hodgkin lymphoma, hypopharyngeal cancer, islet cell tumors, pancreatic neuroendocrine tumors, kidney (renal cell) cancer, laryngeal cancer, leukemia, lip cancer, oral cancer, lung cancer (non-small cell and small cell), lymphoma, melanoma, Merkel cell carcinoma, mesothelioma, metastatic squamous cell neck cancer, midline tract carcinoma with and without NUT gene changes, multiple endocrine neoplasia syndromes, multiple myeloma, plasma cell neoplasms, mycosis fungoides, myelodysplastic syndromes, myelodysplastic/myeloproliferative neoplasms, chronic myelogenous leukemia, nasal cancer, sinus cancer, non-Hodgkin lymphoma, pancreatic cancer, paraganglioma, paranasal sinus cancer, parathyroid cancer, penile cancer, pharyngeal cancer, pheochromocytoma, pituitary cancer, peritoneal cancer, prostate cancer, rectal cancer, Rhabdomyosarcoma, salivary gland cancer, uterine sarcoma, Sézary syndrome, skin cancer, small intestine cancer, large intestine cancer (colon cancer), soft tissue sarcoma, T-cell lymphoma, throat cancer, oropharyngeal cancer, nasopharyngeal cancer, hypopharyngeal cancer, thymoma, thymic carcinoma, thyroid cancer, transitional cell cancer of the renal pelvis and ureter, urethral cancer, uterine cancer, vaginal cancer, cervical cancer, vascular tumors and cancer, vulvar cancer, and Wilms Tumor.

As used herein, “cDNA” refers to a DNA sequence that is complementary to a RNA transcript in a cell. It is a man-made molecule. Typically, cDNA is made in vitro by an enzyme called reverse-transcriptase using RNA transcripts as templates.

As used herein, “chemotherapeutic agent” or “chemotherapeutic” refers to a therapeutic agent utilized to prevent or treat cancer.

As used herein, “control” can refer to an alternative subject or sample used in an experiment for comparison purpose and included to minimize or distinguish the effect of variables other than an independent variable.

As used herein with reference to the relationship between DNA, cDNA, RNA, RNA, protein/peptides, and the like “corresponding to” or “encoding” (used interchangeably herein) refers to the underlying biological relationship between these different molecules. As such, one of skill in the art would understand that operatively “corresponding to” can direct them to determine the possible underlying and/or resulting sequences of other molecules given the sequence of any other molecule which has a similar biological relationship with these molecules. For example, from a DNA sequence an RNA sequence can be determined and from an RNA sequence a cDNA sequence can be determined.

As used herein, “deoxyribonucleic acid (DNA)” and “ribonucleic acid (RNA)” can generally refer to any polyribonucleotide or polydeoxyribonucleotide (collectively polynucleotides), which may be unmodified RNA or DNA or modified RNA or DNA. RNA can be in the form of non-coding RNA such as tRNA (transfer RNA), snRNA (small nuclear RNA), IRNA (ribosomal RNA), anti-sense RNA, RNAi (RNA interference construct), siRNA (short interfering RNA), microRNA (miRNA), or ribozymes, aptamers, guide RNA (gRNA) or coding mRNA (messenger RNA).

As used herein, the terms “disease” or “disorder” are used interchangeably throughout this specification and refer to any alternation in state of the body or of some of the organs, interrupting or disturbing the performance of the functions and/or causing symptoms such as discomfort, dysfunction, distress, or even death to the person afflicted or those in contact with a person. A disease or disorder can also be related to a distemper, ailing, ailment, malady, disorder, sickness, illness, complaint, indisposition, or affliction.

As used herein, “derivative” can refer to any peptide, polypeptide, polynucleotide, having the same or a similar core structure, a functionality, and/or activity, but having at least one structural or sequential (e.g., amino acid or nucleotide) difference.

As used herein, “expression” refers to the process by which polynucleotides are transcribed into RNA transcripts. In the context of mRNA and other translated RNA species, “expression” also refers to the process or processes by which the transcribed RNA is subsequently translated into peptides, polypeptides, or proteins. In some instances, “expression” can also reflect the stability of a given RNA. For example, when one measures RNA, depending on the method of detection and/or quantification of the RNA as well as other techniques used in conjunction with RNA detection and/or quantification, it can be that increased/decreased RNA transcript levels are the result of increased/decreased transcription and/or increased/decreased stability and/or degradation of the RNA transcript. One of ordinary skill in the art will appreciate these techniques and the relation “expression” in these various contexts to the underlying biological mechanisms.

As used herein, “fragment” as used throughout this specification with reference to a peptide, polypeptide, or protein generally denotes a portion of the peptide, polypeptide, or protein, such as typically an N- and/or C-terminally truncated form of the peptide, polypeptide, or protein. Preferably, a fragment may comprise at least about 30%, e.g., at least about 50% or at least about 70%, preferably at least about 80%, e.g., at least about 85%, more preferably at least about 90%, and yet more preferably at least about 95% or even about 99% of the amino acid sequence length of said peptide, polypeptide, or protein. For example, insofar not exceeding the length of the full-length peptide, polypeptide, or protein, a fragment may include a sequence of ≥5 consecutive amino acids, or ≥10 consecutive amino acids, or ≥20 consecutive amino acids, or ≥30 consecutive amino acids, e.g., ≥40 consecutive amino acids, such as for example ≥50 consecutive amino acids, e.g., ≥60, ≥70, ≥80, ≥90, ≥100, ≥200, ≥ 300, ≥400, ≥500 or ≥600 consecutive amino acids of the corresponding full-length peptide, polypeptide, or protein.

The term “fragment” with reference to a nucleic acid (polynucleotide) generally denotes a 5′- and/or 3′-truncated form of a nucleic acid. Preferably, a fragment may comprise at least about 30%, e.g., at least about 50% or at least about 70%, preferably at least about 80%, e.g., at least about 85%, more preferably at least about 90%, and yet more preferably at least about 95% or even about 99% of the nucleic acid sequence length of said nucleic acid. For example, insofar not exceeding the length of the full-length nucleic acid, a fragment may include a sequence of ≥5 consecutive nucleotides, or ≥10 consecutive nucleotides, or ≥20 consecutive nucleotides, or ≥30 consecutive nucleotides, e.g., >40 consecutive nucleotides, such as for example ≥50 consecutive nucleotides, e.g., ≥60, ≥70, ≥80, ≥90, ≥100, ≥200, ≥ 300, ≥400, ≥500 or ≥600 consecutive nucleotides of the corresponding full-length nucleic acid.

The terms encompass fragments arising by any mechanism, in vivo and/or in vitro, such as, without limitation, by alternative transcription or translation, exo-and/or endo-proteolysis, exo- and/or endo-nucleolysis, or degradation of the peptide, polypeptide, protein, or nucleic acid, such as, for example, by physical, chemical and/or enzymatic proteolysis or nucleolysis.

As used herein, “gene” refers to a hereditary unit corresponding to a sequence of DNA that occupies a specific location on a chromosome and that contains the genetic instruction for a characteristic(s) or trait(s) in an organism. The term gene can refer to translated and/or untranslated regions of a genome. “Gene” can refer to the specific sequence of DNA that is transcribed into an RNA transcript that can be translated into a polypeptide or be a catalytic RNA molecule, including but not limited to, tRNA, siRNA, piRNA, miRNA, long-non-coding RNA and shRNA.

As used herein, “identity,” refers to a relationship between two or more nucleotide or polypeptide sequences, as determined by comparing the sequences. In the art, “identity” also refers to the degree of sequence relatedness between polynucleotide or polypeptide sequences as determined by the match between strings of such sequences. “Identity” can be readily calculated by known methods, including, but not limited to, those described in (Computational Molecular Biology, Lesk, A. M., Ed., Oxford University Press, New York, 1988; Biocomputing: Informatics and Genome Projects, Smith, D. W., Ed., Academic Press, New York, 1993; Computer Analysis of Sequence Data, Part I, Griffin, A. M., and Griffin, H. G., Eds., Humana Press, New Jersey, 1994; Sequence Analysis in Molecular Biology, von Heinje, G., Academic Press, 1987; and Sequence Analysis Primer, Gribskov, M. and Devereux, J., Eds., M Stockton Press, New York, 1991; and Carillo, H., and Lipman, D., SIAM J. Applied Math. 1988, 48:1073. Preferred methods to determine identity are designed to give the largest match between the sequences tested. Methods to determine identity are codified in publicly available computer programs. The percent identity between two sequences can be determined by using analysis software (e.g., Sequence Analysis Software Package of the Genetics Computer Group, Madison Wis.) that incorporates the Needelman and Wunsch, (J. Mol. Biol., 1970, 48:443-453,) algorithm (e.g., NBLAST, and XBLAST). The default parameters are used to determine the identity for the polypeptides or polynucleotides of the present disclosure, unless stated otherwise.

As used herein, “immunomodulator,” refers to an agent, such as a therapeutic agent, which is capable of modulating or regulating one or more immune function or response.

As used herein, “modulate” broadly denotes a qualitative and/or quantitative alteration, change or variation in that which is being modulated. Where modulation can be assessed quantitatively—for example, where modulation comprises or consists of a change in a quantifiable variable such as a quantifiable property of a cell or where a quantifiable variable provides a suitable surrogate for the modulation—modulation specifically encompasses both increase (e.g., activation) or decrease (e.g., inhibition) in the measured variable. The term encompasses any extent of such modulation, e.g., any extent of such increase or decrease, and may more particularly refer to statistically significant increase or decrease in the measured variable. By means of example, in aspects modulation may encompass an increase in the value of the measured variable by about 10 to 500 percent or more. In aspects, modulation can encompass an increase in the value of at least 10%, 20%, 30%, 40%, 50%, 75%, 100%, 150%, 200%, 250%, 300%, 400% to 500% or more, compared to a reference situation or suitable control without said modulation. In aspects, modulation may encompass a decrease or reduction in the value of the measured variable by about 5 to about 100%. In some embodiments, the decrease can be about 5%, 10%, 20%, 30%, 40%, 50%, 60%, 70%, 80%, 90%, 95%, 96%, 97%, 98%, 99% to about 100%, compared to a reference situation or suitable control without said modulation. In aspects, modulation may be specific or selective, hence, one or more desired phenotypic aspects of a cell or cell population may be modulated without substantially altering other (unintended, undesired) phenotypic aspect(s).

As used herein, “nucleic acid,” “nucleotide sequence,” and “polynucleotide” can be used interchangeably herein and can generally refer to a string of at least two base-sugar-phosphate combinations and refers to, among others, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, polynucleotide as used herein can refer to triple-stranded regions comprising RNA or DNA or both RNA and DNA. The strands in such regions can be from the same molecule or from different molecules. The regions may include all of one or more of the molecules, but more typically involve only a region of some of the molecules. One of the molecules of a triple-helical region often is an oligonucleotide. “Polynucleotide” and “nucleic acids” also encompasses such chemically, enzymatically or metabolically modified forms of polynucleotides, as well as the chemical forms of DNA and RNA characteristic of viruses and cells, including simple and complex cells, inter alia. For instance, the term polynucleotide as used herein can include DNAs or RNAs as described herein that contain one or more modified bases. Thus, DNAs or RNAs including unusual bases, such as inosine, or modified bases, such as tritylated bases, to name just two examples, are polynucleotides as the term is used herein. “Polynucleotide”, “nucleotide sequences” and “nucleic acids” also includes PNAs (peptide nucleic acids), phosphorothioates, and other variants of the phosphate backbone of native nucleic acids. Natural nucleic acids have a phosphate backbone, artificial nucleic acids can contain other types of backbones, but contain the same bases. Thus, DNAs or RNAs with backbones modified for stability or for other reasons are “nucleic acids” or “polynucleotides” as that term is intended herein. As used herein, “nucleic acid sequence” and “oligonucleotide” also encompasses a nucleic acid and polynucleotide as defined elsewhere herein.

As used herein “peptide” can refer to chains of at least 2 amino acids that are short, relative to a protein or polypeptide.

As used herein, “pharmaceutical formulation” refers to the combination of an active agent, compound, or ingredient with a pharmaceutically acceptable carrier or excipient, making the composition suitable for diagnostic, therapeutic, or preventive use in vitro, in vivo, or ex vivo.

As used herein, “pharmaceutically acceptable carrier or excipient” refers to a carrier or excipient that is useful in preparing a pharmaceutical formulation that is generally safe, non-toxic, and is neither biologically or otherwise undesirable, and includes a carrier or excipient that is acceptable for veterinary use as well as human pharmaceutical use. A “pharmaceutically acceptable carrier or excipient” as used in the specification and claims includes both one and more than one such carrier or excipient.

As used herein, “pharmaceutically acceptable salt” refers to any acid or base addition salt whose counter-ions are non-toxic to the subject to which they are administered in pharmaceutical doses of the salts.

As used herein, a “population” of cells is any number of cells greater than 1, but is preferably at least 1×10³ cells, at least 1×10⁴ cells, at least at least 1×10⁵ cells, at least 1×10⁶ cells, at least 1×10⁷ cells, at least 1×10⁸ cells, at least 1×10⁹ cells, or at least 1×10¹⁰ cells.

As used herein, “polypeptides” or “proteins” refers to amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V). “Protein” and “Polypeptide” can refer to a molecule composed of one or more chains of amino acids in a specific order. The term protein is used interchangeable with “polypeptide.” The order is determined by the base sequence of nucleotides in the gene coding for the protein. Proteins can be required for the structure, function, and regulation of the body's cells, tissues, and organs.

As used herein, the terms “recombinant” or “engineered” generally refer to a non-naturally occurring nucleic acid, nucleic acid construct, or polypeptide. Such non-naturally occurring nucleic acids may include natural nucleic acids that have been modified, for example that have deletions, substitutions, inversions, insertions, etc., and/or combinations of nucleic acid sequences of different origin that are joined using molecular biology technologies (e.g., a nucleic acid sequences encoding a fusion protein (e.g., a protein or polypeptide formed from the combination of two different proteins or protein fragments), the combination of a nucleic acid encoding a polypeptide to a promoter sequence, where the coding sequence and promoter sequence are from different sources or otherwise do not typically occur together naturally (e.g., a nucleic acid and a constitutive promoter), etc. Recombinant or engineered can also refer to the polypeptide encoded by the recombinant nucleic acid. Non-naturally occurring nucleic acids or polypeptides include nucleic acids and polypeptides modified by man.

As used herein, the term “specific binding” refers to non-covalent physical association of a first and a second moiety wherein the association between the first and second moieties is at least 2 times as strong, at least 5 times as strong as, at least 10 times as strong as, at least 50 times as strong as, at least 100 times as strong as, or stronger than the association of either moiety with most or all other moieties present in the environment in which binding occurs. Binding of two or more entities may be considered specific if the equilibrium dissociation constant, Kd, is 10⁻³ M or less, 10⁻⁴ M or less, 10⁻⁵ M or less, 10⁻⁶ M or less, 10⁻⁷ M or less, 10⁻⁸ M or less, 10⁻⁹ M or less, 10⁻¹⁰ M or less, 10⁻¹¹ M or less, or 10⁻¹² M or less under the conditions employed, e.g., under physiological conditions such as those inside a cell or consistent with cell survival. In some embodiments, specific binding can be accomplished by a plurality of weaker interactions (e.g., a plurality of individual interactions, wherein each individual interaction is characterized by a Kd of greater than 10⁻³ M). In some embodiments, specific binding, which can be referred to as “molecular recognition,” is a saturable binding interaction between two entities that is dependent on complementary orientation of functional groups on each entity. Examples of specific binding interactions include primer-polynucleotide interaction, aptamer-aptamer target interactions, antibody-antigen interactions, avidin-biotin interactions, ligand-receptor interactions, metal-chelate interactions, hybridization between complementary nucleic acids, etc.

As used herein, “substantial” and “substantially,” specify an amount of between 95% and 100%, inclusive, between 96% and 100%, inclusive, between 97% and 100%, inclusive, between 98% and 100%, inclusive, or between 99% and 100%, inclusive.

As used herein, “substantially free” can mean an object species is present at non-detectable or trace levels so as not to interfere with the properties of a composition or process.

As used herein, “substantially pure” can mean an object species is the predominant species present (i.e., on a molar basis it is more abundant than any other individual species in the composition), and preferably a substantially purified fraction is a composition wherein the object species comprises about 50 percent of all species present. Generally, a substantially pure composition will comprise more than about 80 percent of all species present in the composition, more preferably more than about 85%, 90%, 95%, and 99%. Most preferably, the object species is purified to essential homogeneity (contaminant species cannot be detected in the composition by conventional detection methods) wherein the composition consists essentially of a single species.

As used herein, “tangible medium of expression” refers to a medium that is physically tangible or accessible and is not a mere abstract thought or an unrecorded spoken word. “Tangible medium of expression” includes, but is not limited to, words on a cellulosic or plastic material, or data stored in a suitable computer readable memory form. The data can be stored on a unit device, such as a flash memory or CD-ROM or on a server that can be accessed by a user via, e.g., a web interface.

As used herein, “targeting moiety” refers to molecules, complexes, agents, and the like that is capable of specifically or selectively interacting with, binding with, acting on or with, or otherwise associating or recognizing a target molecule, agent, and/or complex that is associated with, part of, coupled to, another object, complex, surface, and the like, such as a cell or cell population, tissue, organ, subcellular locale, object surface, particle etc. Targeting moieties can be chemical, biological, metals, polymers, or other agents and molecules with targeting capabilities. Targeting moieties can be amino acids, peptides, polypeptides, nucleic acids, polynucleotides, lipids, sugars, metals, small molecule chemicals, combinations thereof, and the like. Targeting moieties can be antibodies or fragments thereof, aptamers, DNA, RNA such as guide RNA for a RNA guided nuclease or system, ligands, substrates, enzymes, combinations thereof, and the like. The specificity or selectivity of a targeting moiety can be determined by any suitable method or technique that will be appreciated by those of ordinary skill in the art. For example, in some embodiments, the methods described herein include determining the disassociation constant for the targeting moiety and target. In some embodiments, the targeting moiety has a specificity the equilibrium dissociation constant, Kd, is 10⁻³ M or less, 10⁻⁴ M or less, 10⁻⁵ M or less, 10⁻⁶ M or less, 10⁻⁷ M or less, 10⁻⁸ M or less, 10⁻⁹ M or less, 10⁻¹⁰ M or less, 10⁻¹¹ M or less, or 10⁻¹² M or less under the conditions employed, e.g., under physiological conditions such as those inside a cell or consistent with cell survival. In some embodiments, specific binding can be accomplished by a plurality of weaker interactions (e.g., a plurality of individual interactions, wherein each individual interaction is characterized by a Kd of greater than 10⁻³ M). In some embodiments, the targeting moiety has increased binding with, association with, interaction with, activity on as compared to non-targets, such as a 1 to 500 or more fold increase. Targets of targeting moieties can be amino acids, peptides, polypeptides, nucleic acids, polynucleotides, lipids, sugars, metals, small molecule chemicals, combinations thereof, and the like. Targets can be receptors, biomarkers, transporters, antigens, complexes, combinations thereof, and the like.

As used herein, “therapeutic” refers to treating, healing, and/or ameliorating a disease, disorder, condition, or side effect, or to decreasing in the rate of advancement of a disease, disorder, condition, or side effect. A “therapeutically effective amount” can therefore refer to an amount of a compound that can yield a therapeutic effect.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof, such as an SCN1B mediate disease. The effect can be therapeutic in terms of a partial or complete cure of a disease, condition, symptom or adverse effect attributed to the disease, disorder, or condition. The term “treatment” as used herein covers any treatment of a cardiac arrythmia and/or other cardiac contractility/fibrillation disorder and/or dysfunction, an epilepsy, a neurodegenerative disease (including, but not limited to, Alzheimer's Disease, dementias, and/or the like), a neuropathy, pain, cancer (including but not limited to cervical cancer, breast cancer, and prostate cancer), an Autism spectrum disorder, a mood disorder, or any combination thereof, in a subject, particularly a human or non-human animal or avian, and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, such as treating the pain of a subject by administration of an analgesic agent even though such agent does not treat the cause of the pain.

As used herein, “Autism Spectrum disorders” refers to a group of developmental disorders caused by differences in the brain that affects how a person interacts with others, communicates, learns, and behaves. See e.g., Lord et al., Lancet. 2018 Aug. 11;392 (10146): 508-520; Manoli and State. Am J Psychiatry. 2021 Jan. 1;178 (1): 30-38; Kodak and Bergmann. Pediatr Clin North Am. 2020 June;67 (3): 525-535.

As used herein, “neurodegenerative disease” refers to any disease or disorder whose etiology or pathology involves neurodegeneration. Exemplary neurodegenerative diseases include, but are not limited to Alzheimer's Disease, Parkinson's disease, Huntington's disease, amyotrophic lateral sclerosis, frontotemporal dementia and the spinocerebellar ataxias. See e.g., Gitler et al., Dis Model Mech. 2017 May 1;10 (5): 499-502, Armstrong, R. Folia Neuropathol. 2020; 58 (2): 93-112.

As used herein, “epilepsy” includes focal epilepsy, generalized epilepsy, or both, and unknown. The term also includes epilepsy that is secondary to other conditions, diseases, or disorders.

As used herein in the context of polynucleotides and polypeptides, “variant” refers to a polynucleotide or polypeptide that differs from a reference polynucleotide or polypeptide, but retains essential and/or characteristic properties (structural and/or functional) of the reference polynucleotide or polypeptide. A typical variant of a polypeptide differs in amino acid sequence from another, reference polypeptide. The differences can be limited so that the sequences of the reference polypeptide and the variant are closely similar overall and, in many regions, identical. A variant and reference polypeptide may differ in nucleic or amino acid sequence by one or more modifications at the sequence level or post-transcriptional or post-translational modifications (e.g., substitutions, additions, deletions, methylation, glycosylations, etc.). A substituted nucleic acid may or may not be an unmodified nucleic acid of adenine, thiamine, guanine, cytosine, uracil, including any chemically, enzymatically or metabolically modified forms of these or other nucleotides. A substituted amino acid residue may or may not be one encoded by the genetic code. A variant of a polypeptide may be naturally occurring such as an allelic variant, or it may be a variant that is not known to occur naturally. “Variant” includes functional and structural variants.

As used herein, “wild-type” is the average form of an organism, variety, strain, gene, protein, or characteristic as it occurs in a given population in nature, as distinguished from mutant forms that may result from selective breeding, recombinant engineering, and/or transformation with a transgene.

As used herein, the terms “weight percent,” “wt %,” and “wt. %,” which can be used interchangeably, indicate the percent by weight of a given component based on the total weight of a composition of which it is a component, unless otherwise specified. That is, unless otherwise specified, all wt % values are based on the total weight of the composition. It should be understood that the sum of wt % values for all components in a disclosed composition or formulation are equal to 100. Alternatively, if the wt % value is based on the total weight of a subset of components in a composition, it should be understood that the sum of wt % values the specified components in the disclosed composition or formulation are equal to 100.

All publications, published patent documents, and patent applications cited herein are hereby incorporated by reference to the same extent as though each individual publication, published patent document, or patent application was specifically and individually indicated as being incorporated by reference.

### Overview

Voltage-gated sodium channels (VGSCs) are responsible for initiation and propagation of action potentials in excitable cells. Mutations in the genes encoding β subunits are linked to a number of diseases, including epilepsy, sudden death syndromes like SUDEP and SIDS, and cardiac arrhythmia. Currently there are no β subunit-specific therapeutics available. As such, there exists a need for therapeutics directed to voltage-gated sodium channels, and more particularly the β subunit.

Generally, mammalian VGSCs are heterotrimeric complexes of a and β subunits. Originally called “auxiliary,” we now know that β subunit proteins are multifunctional signaling molecules that play roles in both excitable and non-excitable cell types, and with or without the pore-forming a subunit present. β subunits function in VGSC and potassium channel modulation, cell adhesion, and gene regulation. VGSCs are responsible for generating the rising phase and propagation of the action potential (AP) in excitable cells, including neurons, and cardiac myocytes. They also play various roles in non-excitable cells. In short, VGSCs participate in diverse and essential roles throughout multiple tissues and systems. Thus, function (or dysfunction) of VGSCs play key roles in normal health and physiology as well as contribute to the pathophysiology of various diseases.

With respect to the beta subunit proteins, five VGSC beta subunit proteins are encoded by a family of four genes. β1 and its splice variant BIB are encoded by SCN1B (see e.g., Isom et al., Science. 1992; 256:839-42; Kazen-Gillespie et al., J Biol Chem. 2000; 275:1079-88; Patino et al., J Neurosci. 2011; 31:14577-91; and Quin et al., Eur J Biochem. 2003; 270:4762-70). With the exception of BIB, β subunits have type 1 topology, consisting of a single polypeptide chain with an extracellular N-terminus, a single transmembrane-spanning segment, and an intracellular C-terminus. In the case of β1B (originally called β1A), normal splicing of the exon 3/intron 3 boundary does not occur, leading to in-frame retention of intron 3 and generation of an alternate C-terminal sequence that does not include a transmembrane domain. As a result, β1B is a developmentally regulated secreted protein.

VGSC α and β subunits interact via two distinct mechanisms. In the mechanism involving β1, β1 and β3 interact non-covalently with a subunits via their N-and C-termini. BIB selectively associates with Nav1.5., but not Nav1.1 or Nav1.3. β2 and β4 engage in covalent interactions with a subunits via a single N-terminal cysteine in the extracellular Ig loop.

VGSC β subunits are expressed in a wide range of tissue and cell types, including excitable and non-excitable cells, and their expression patterns vary with development. In general, β1 is abundantly expressed in skeletal muscle, heart and brain, while β1B is highly expressed in brain and skeletal muscle and present at a very low level in heart, placenta, lung, liver, kidney and pancreas. For example, in mammalian brain, β1 and β2 predominate in postnatal development with peak levels in adult, while expression of β1B and β3 are higher in embryonic development and early life. This developmental expression pattern is different in heart, where β1B and β3 expression persist into adulthood. See e.g., Table I of O'Malley and Isom. Annu Rev Physiol. 2015. 77:481-504 for tissue expression of different subunits in different cell types of the nervous system and cardiac tissue.

VGSC β subunits function in concert with a subunits to promote channel trafficking to the plasma membrane and to modulate VGSC biophysical properties. β subunits also modulate a subunit function, with effects including alterations in peak INa density, voltage-dependence of activation and inactivation, rate of inactivation, and persistent and resurgent INa in cell- and tissue-specific patterns. β subunits, particularly β1, also interact with and functionally modulate some VGKCs.

β subunits, including β1, are cell adhesion molecules and are classified in the Ig super family of cell adhesion molecules. In vitro, β1 engages in extracellular homophilic cell adhesion as well as heterophilic adhesive interactions with VGSC β2, contactin-1, Nf-186, Nf-155, NrCAM, N-cadherin, and the extracellular matrix protein tenascin-R. It is predicted that β1B, which shares the N-terminal domain including the Ig loop with β1, engage in similar adhesive interactions as β1. Because β1B is a secreted molecule that can participate in long distance adhesive interactions, it may play roles in novel signaling functions in development, including acting as a soluble antagonist for cell adhesive interactions between cells.

β subunit-mediated adhesion plays a role in multiple physiological and developmental processes. For example, adhesion is critical in axonal fasciculation and axon pathfinding during development.

To summarize, VGSC β subunits are multifunctional, with the capacity to signal through multiple pathways in multiple tissues on multiple timescales. A growing number of β subunit gene mutations have been linked to various neuronal and cardiac diseases. Yet, therapeutics targeting VGSCs, and more particularly the β subunits, have yet to reach clinical relevance. As such there exists a need for compositions, particularly therapeutically relevant compositions, that target VGSCs, and more particularly the β subunits, for treating VGSC medicated disease, disorders, and conditions.

With that said, embodiments disclosed herein can provide SCN1B mimetic peptides and formulations thereof. In some embodiments, the SCN1B mimetic peptides can modulate VGSC β1 subunit function and/or activity and thus activity of VGSC and/or other cells or complexes they interact with (e.g., VGKCs). Also described in certain embodiments herein are methods of administering the SCN1B mimetic peptides and formulations thereof to a subject in need thereof, such as a subject having or suspected of having a disease, condition, or disorder whose pathophysiology involves VGSC, VGKC, or other molecules that interacts with SCN1B. In some embodiments, the disease, disorder, or condition is a cardiac disease, disorder, or condition (e.g., arrythmia) or a brain or neuron disease, disorder, or condition (e.g., epilepsy, pain, neuropathy, neurodegenerative and demyelinating diseases, dementia, Alzheimer's Disease), cancer (including but not limited to cervical cancer, breast cancer, and prostate cancer), and others (e.g., Autism spectrum and other mood disorders).

Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

### Scn1B Mimetic Peptides and Encoding Polynucleotides

### SCN1B Mimetic Peptides

Described in several embodiments herein are SCN1B mimetic peptides. The term “SCN1B mimetic peptide” refers to peptides that sequentially, conformationally, and/or functionally mimics the sequence, three dimensional or other conformation (e.g., dimerization), and/or one or more biological functions of the native full length SCN1B mimetic peptide or a functional domain thereof. In some embodiments, the SCN1B mimetic peptides are monomers. In some embodiments, the SCN1B mimetic peptides are dimers or are capable of dimerizing. In some embodiments the dimer SCN1B mimetic peptides are homodimers. Without being bound by theory, the SCN1B mimetic peptides are capable of binding to, interacting with, or otherwise associating with beta subunits and other beta subunit interaction domains. In some embodiments, the SCN1B mimetic peptides inhibit the native SCN1B from interacting with an alpha or beta subunit of a VGSC, VGKC, or other molecule or complex that a native SCN1B interacts with.

In some embodiments, the SCN1B mimetic peptides have a sequence that is about 95-100 percent identical to region of an SCN1B polypeptide that is 2-30 amino acids in length. In some embodiments, the SCN1B mimetic peptides have a sequence that is about 95-100 percent identical to region of an SCN1B isoform 1 polypeptide that is 2-30 amino acids in length. In some embodiments, the SCN1B mimetic peptides have a sequence that is about 95-100 percent identical to region of an SCN1B isoform 2 polypeptide that is 2-30 amino acids in length.

In some embodiments, the SCN1B mimetic peptide has a sequence that includes or is composed only of amino acids 67-86 of SCN1B isoform 1 (SEQ ID NO: 11) or isoform 2 (SEQ ID NO: 12). In some embodiments the SCN1B mimetic peptide has a sequence that includes or is composed only of SEQ ID NO: 2. In some embodiments, the SCN1B mimetic peptide is a dimer composed of one or two monomers of SEQ ID NO: 2. In some embodiments, the dimer includes one or more amino acids separating the two monomers. In some embodiments the dimer is a cyclic dimer. In some embodiments, the SCN1B mimetic peptide has a sequence that includes or is composed only of SEQ ID NO: 3.

In some embodiments, the SCN1B mimetic peptide has a sequence that includes or is composed only of amino acids 78-86 of SCN1B isoform 1 (SEQ ID NO: 11) or isoform 2 (SEQ ID NO: 12). In some embodiments the SCN1B mimetic peptide has a sequence that includes or is composed only of SEQ ID NO: 10. In some embodiments, the SCN1B mimetic peptide is a dimer composed of one or two monomers of SEQ ID NO: 10. In some embodiments, the dimer includes one or more amino acids separating the two monomers. In some embodiments the dimer is a cyclic dimer. In some embodiments, the SCN1B mimetic peptide has a sequence that includes or is composed only of SEQ ID NO: 2, SEQ ID NO: 3, SEQ ID NO: 5 or SEQ ID NO: 6.

In some embodiments, the SCN1B mimetic peptide has a sequence that includes or is composed only of amino acids 80-86 of SCN1B isoform 1 (SEQ ID NO: 11) or isoform 2 (SEQ ID NO: 12). In some embodiments the SCN1B mimetic peptide has a sequence that includes or is composed only of SEQ ID NO: 7. In some embodiments, the SCN1B mimetic peptide is a dimer composed of one or two monomers of SEQ ID NO: 7. In some embodiments, the dimer includes one or more amino acids separating the two monomers. In some embodiments the dimer is a cyclic dimer. In some embodiments, the SCN1B mimetic peptide has a sequence that includes or is composed only of SEQ ID NO: 2, SEQ ID NO: 3, SEQ ID NO: 5, or SEQ ID NO: 6.

In some embodiments, the SCN1B mimetic peptide has a sequence that includes or is composed only of amino acids 78-83 of SCN1B isoform 1 (SEQ ID NO: 11) or isoform 2 (SEQ ID NO: 12). In some embodiments the SCN1B mimetic peptide has a sequence that includes or is composed only of SEQ ID NO: 4. In some embodiments, the SCN1B mimetic peptide is a dimer composed of one or two monomers of SEQ ID NO: 4. In some embodiments, the dimer includes one or more amino acids separating the two monomers. In some embodiments the dimer is a cyclic dimer. In some embodiments, the SCN1B mimetic peptide has a sequence that includes or is composed only of SEQ ID NO: 2, SEQ ID NO: 3, SEQ ID NO: 5 or SEQ ID NO: 6.

In some embodiments, the SCN1B mimetic peptide has a sequence that includes or is composed only of amino acids 69-75 of SCN1B isoform 1 (SEQ ID NO: 11) or isoform 2 (SEQ ID NO: 12). In some embodiments the SCN1B mimetic peptide has a sequence that includes or is composed only of SEQ ID NO: 8. In some embodiments, the SCN1B mimetic peptide is a dimer composed of one or two monomers of SEQ ID NO: 8. In some embodiments, the dimer includes one or more amino acids separating the two monomers. In some embodiments the dimer is a cyclic dimer. In some embodiments, the SCN1B mimetic peptide has a sequence that includes or is composed only of SEQ ID NO: 2 or SEQ ID NO: 3.

In some embodiments, the SCN1B mimetic peptide has a sequence that includes or is composed only of amino acids 69-75 of SCN1B isoform 1 (SEQ ID NO: 11) or isoform 2 (SEQ ID NO: 12). In some embodiments the SCN1B mimetic peptide has a sequence that includes or is composed only of SEQ ID NO: 8. In some embodiments, the SCN1B mimetic peptide is a dimer composed of one or two monomers of SEQ ID NO: 8. In some embodiments, the dimer includes one or more amino acids separating the two monomers. In some embodiments the dimer is a cyclic dimer. In some embodiments, the SCN1B mimetic peptide has a sequence that includes or is composed only of SEQ ID NO: 2 or SEQ ID NO: 3.

In some embodiments, the SCN1B mimetic peptide has a sequence that includes or is composed only of amino acids 73-79 of SCN1B isoform 1 (SEQ ID NO: 11) or isoform 2 (SEQ ID NO: 12). In some embodiments the SCN1B mimetic peptide has a sequence that includes or is composed only of SEQ ID NO: 9. In some embodiments, the SCN1B mimetic peptide is a dimer composed of one or two monomers of SEQ ID NO: 9. In some embodiments, the dimer includes one or more amino acids separating the two monomers. In some embodiments the dimer is a cyclic dimer. In some embodiments, the SCN1B mimetic peptide has a sequence that includes or is composed only of SEQ ID NO: 2 or SEQ ID NO: 3.

SEQ ID NO: 1
QKGTEEFVKILRYENEVLQLEEDERFEGRV
SEQ ID NO: 2
FVKILRYENEVLQLEEDERF (βadp1)
SEQ ID NO: 3
FVKILRYENEVLQLEEDERGFVKILRYENEVLQLEEDER
(Dbl-βadp1)
SEQ ID NO: 4
LQLEED
SEQ ID NO: 5
LQLEEDERFGLQLEEDERF
SEQ ID NO: 6
CLQLEEDERFGLQLEEDERFC
SEQ ID NO: 7
LEEDERF
SEQ ID NO: 8
KILRYEN
SEQ ID NO: 9
YENEVLQ
SEQ ID NO: 10
LQLEEDERF
SCN1B Isoform 1 (underlined region is βadp1
(SEQ ID NO: 2))
>sp|Q07699|SCN1B_HUMAN Sodium channel subunit
beta-1 OS = Homo sapiens OX = 9606
GN = SCN1B PE = 1 SV = 1
(SEQ ID NO: 11)
M₁GRLLALVVGAALVSSACGGCVEVDSETEAVYGMTFKILCISCKRRSE
TNAETFTEWTFRQKGTEEF₆₇VK₆₉ILRY₇₃ENEVL₇₈QL₈₀EED₈₃ER
F₈₆EGRVVWNGSRGTKDLQDLSIFITNVTYNHSGDYECHVYRLLFFENY
EHNTSVVKKIHIEVVDKANRDMASIVSEIMMYVLIVVLTIWLVAEMIYC
YKKIAAATETAAQENASEYLAITSESKENCTGVQVAE
SCN1B Isoform 2 - splice variant (underlined
region is βadp1 (SEQ ID NO: 2))
>sp|Q07699-2|SCN1B_HUMAN Isoform 2 of Sodium
channel subunit beta-1 OS = Homo sapiens
OX = 9606 GN = SCN1B
(SEQ ID NO: 12)
M₁GRLLALVVGAALVSSACGGCVEVDSETEAVYGMTFKILCISCKRRSE
TNAETFTEWTFRQKGTEEEF₆₇VK₆₉ILRY₇₃ENEVL₇₈QL₈₀EED₈₃ER
F₈₆EGRVVWNGSRGTKDLQDLSIFITNVTYNHSGDYECHVYRLLFFENY
EHNTSVVKKIHIEVVDKGESGAACPFTVTHRRARWRDRWQAVDRTGWLC
AWPANRPQQRAEGEGSSPSCPLQLWPLFLSSPRRGQSMPVPHRRSGYRT
QLCHLCCMTSGRCLLSLSQRVVLGLPGIIIRCVSRGVV

In some embodiments, the SCN1B mimetic peptide is a homolog or ortholog to an SCN1B isoform (see above) that contains an immunoglobulin domain with the adhesion sequence mimetic corresponding to βadp1. Exemplary SCN1B mimetic peptides that are homologs or orthologs to SCN1B that contains immunoglobulin domain with an adhesion sequence mimetic corresponding to βadp1 are as follows. The adhesion mimetic based on these sequences and up to 30 amino acids to the N terminus and C terminus of these sequences have potential as beta adhesion modulating peptides, both in single and double forms. Without being bound by theory, it is believed that homologues and orthologues containing an N-terminal sequence mimetic corresponding to βadp1 should be effective in mediate adhesion targeting as per βadp1.

In some embodiments, the SCN1B mimetic peptide is a homolog or ortholog is SEQ ID NO: 38, 40, or 42. In some embodiments, the SCN1B mimetic peptide is a βadp1 homology peptide as identified in Table 1 below.

In some embodiments, the SCN1B mimetic peptides have a sequence that is about 95-100 percent identical to region of a polypeptide according to any one of SEQ ID NO: 37-42 that is 2-30 amino acids in length.

In some embodiments, the SCN1B mimetic peptide has a sequence that includes or is composed only of amino acids 67-98 of SCN1B isoform 1 (SEQ ID NO: 37), amino acids 62 to 93 of SEQ ID NO: 39, or amino acids 68-102 of SEQ ID NO: 41. In some embodiments the SCN1B mimetic peptide has a sequence that includes or is composed only of SEQ ID NO: 38, 40, or 42. In some embodiments, the SCN1B mimetic peptide is a dimer composed of one or two monomers of SEQ ID NO: 38, 40 or 42. In some embodiments, the dimer includes one or more amino acids separating the two monomers. In some embodiments the dimer is a cyclic dimer.

In some embodiments, the SCN1B mimetic peptide has a sequence that includes or is composed only of a peptide identified in Table 1. In some embodiments, the SCN1B mimetic peptide is a dimer composed of one or two monomers of a peptide identified in Table 1. In some embodiments, the dimer includes one or more amino acids separating the two monomers. In some embodiments the dimer is a cyclic dimer.

Beta2 Human
SEQ ID NO: 37
M₁HRDAWLPRPAFSLTGLSLFFSLVPPGRSMEVTVPATLNVLNGSDARLP
CTFNSCYTVNHKQFSLNW₆₇TYQECNNCSEEMFLQFRMKIINLKLERFQD
R₉₈VEFSGNPSKYDVSVMLRNVQPEDEGIYNCYIMNPPDRHRGHGKIHLQ
VLMEEPPERDSTVAVIVGASVGGFLAVVILVLMVVKCVRRKKEQKLSTDD
LKTEEEGKTDGEGNPDDGAK
SEQ ID NO: 38
WTYQ ECNNCSEEMF LQFRMKIINL
Beta3 Human
SEQ ID NO: 39
M₁PAFNRLFPLASLVLIYWVSVCFPVCVEVPSETEAVQGNPMKLRCISCM
KREEVEATTVVEW₆₂FYRPEGGKDFLIYEYRNGHQEVESPFQGRLQ₉₃WN
GSKDLQDVSITVLNVTLNDSGLYTCNVSREFEFEAHRPFVKTTRLIPLRV
TEEAGEDFTSVVSEIMMYILLVFLTLWLLIEMIYCYRKVSKAEEAAQENA
SDYLAIPSENKENSAVPVEE
SEQ ID NO: 40
WFYRPEGGKDFLIYEYRNGHQEVESPFQGRLQ
Beta4 SCN4B_HUMAN
SEQ ID NO: 41
M₁PGAGDGGKAPARWLGTGLLGLFLLPVTLSLEVSVGKATDIYAVNGTEI
LLPCTFSSCFGFEDLHFRW₆₈TYNSSDAFKILIEGTVKNEKSDPKVTLKD
DDRIT₁₀₂LVGSTKEKMNNISIVLRDLEFSDTGKYTCHVKNPKENNLQHH
ATIFLQVVDRLEEVDNTVTLIILAVVGGVIGLLILILLIKKLIIFILKKT
REKKKECLVSSSGNDNTENGLPGSKAEEKPPSKV
SEQ ID NO: 42
WTYNSSDAFKILIEGTVKNEKSDPKVTLKDDDRIT

TABLE 1

| UniProtKB | | Match hit | |
|---|---|---|---|
| Entry ID | Protein names | 100806040200 | Identity |

| | | |
|---|---|---|
| Q00954 | Sodium channel subunit beta-1 (Rattus norvegicus) | 100.0% |
| P53788 | Sodium channel subunit beta-1 (Oryctolagus cuniculus) | 100.0% |
| P97952 | Sodium channel subunit beta-1 (Mus musculus) | 100.0% |
| Q4PPC4 | Sodium channel subunit beta-1 (Canis lupus familiaris) | 100.0% |
| L5K5K4 | Sodium channel subunit beta-1 (Pteropus alecto) | 100.0% |
| A0A6P6BY27 | Sodium channel subunit beta-1 (Pteropus vampyrus) | 100.0% |
| A0A4V5P920 | Sodium channel subunit beta-1 (Monodon monoceros) | 100.0% |
| A0A7N5K7J3 | Sodium channel subunit beta-1 (Ailuropoda melanoleuca) | 100.0% |
| A0A287D7T2 | Sodium channel subunit beta-1 (Ictidomys tridecemlineatus) | 100.0% |
| A0A2K5JFC1 | Sodium channel subunit beta-1 (Colobus angolensis palliatus) | 100.0% |
| A0A2K5YZ97 | Sodium channel subunit beta-1 (Mandrillus leucophaeus) | 100.0% |
| A0A2Y9PA68 | Sodium channel subunit beta-1 (Delphinapterus leucas) | 100.0% |
| A0A383YQL1 | Sodium channel subunit beta-1 (Balaenoptera acutorostrata s . . . ) | 100.0% |
| A0A2K5V270 | Sodium channel subunit beta-1 (Macaca fascicularis) | 100.0% |
| D2HFQ2 | Sodium channel subunit beta-1 (Ailuropoda melanoleuca) | 100.0% |
| A0A5F4D3H7 | Sodium channel subunit beta-1 (Canis lupus familiaris) | 100.0% |
| A0A6J3QEQ0 | Sodium channel subunit beta-1 (Tursiops truncatus) | 100.0% |
| A0A455ACL2 | Sodium channel subunit beta-1 (Physeter macrocephalus) | 100.0% |
| A0A1D5R912 | Sodium channel subunit beta-1 (Macaca mulatta) | 100.0% |
| A0A643CH93 | Sodium channel subunit beta-1 (Balaenoptera physalus) | 100.0% |
| I3N5M4 | Sodium channel subunit beta-1 (Ictidomys tridecemlineatus) | 100.0% |
| A0A452G6C2 | Sodium channel subunit beta-1 (Capra hircus) | 100.0% |
| A0A2K6SIW4 | Sodium channel subunit beta-1 (Saimiri boliviensis bolivien . . . ) | 100.0% |
| G1T944 | Sodium channel subunit beta-1 (Oryctolagus cuniculus) | 100.0% |
| A0A2K5JFA0 | Sodium channel subunit beta-1 (Colobus angolensis palliatus) | 100.0% |
| A0A2K5YZA2 | Sodium channel subunit beta-1 (Mandrillus leucophaeus) | 100.0% |
| A0A2K6RI10 | Sodium channel subunit beta-1 (Rhinopithecus roxellana) | 100.0% |
| A0A2K5YZC0 | Sodium channel subunit beta-1 (Mandrillus leucophaeus) | 100.0% |
| A0A2K5CI48 | Sodium channel subunit beta-1 (Aotus nancymaae) | 100.0% |
| A0A1D5R7W0 | Sodium channel subunit beta-1 (Macaca mulatta) | 100.0% |
| A0A2K6RI14 | Sodium channel subunit beta-1 (Rhinopithecus roxellana) | 100.0% |
| A0A2K6SJ03 | Sodium channel subunit beta-1 (Saimiri boliviensis bolivien ... ) | 100.0% |
| A0A6P3FG76 | Sodium channel subunit beta-1 (Octodon degus) | 100.0% |
| A0A2Y9P7U3 | Sodium channel subunit beta-1 (Delphinapterus leucas) | 100.0% |
| M3Y7G8 | Sodium channel subunit beta-1 (Mustela putorius furo) | 100.0% |
| A0A2K6GTN6 | Sodium channel subunit beta-1 (Propithecus coquereli) | 100.0% |
| A0A485NUX6 | Sodium channel subunit beta-1 (Lynx pardinus) | 100.0% |
| A0A2F0AYM4 | Sodium channel subunit beta-1 (Eschrichtius robustus) | 100.0% |
| A0A452R1Z0 | Sodium channel subunit beta-1 (Ursus americanus) | 100.0% |
| A0A2K6DUH9 | Sodium channel subunit beta-1 (Macaca nemestrina) | 100.0% |
| A0A2K6LGZ8 | Sodium channel subunit beta-1 (Rhinopithecus bieti) | 100.0% |
| A0A6J3FHR8 | Sodium channel subunit beta-1 (Sapajus apella) | 100.0% |
| A0A2K5V251 | Sodium channel subunit beta-1 (Macaca fascicularis) | 100.0% |
| A0A667I8Q0 | Sodium channel subunit beta-1 (Lynx canadensis) | 100.0% |
| G1L002 | Sodium channel subunit beta-1 (Ailuropoda melanoleuca) | 100.0% |
| A0A341AJG5 | Sodium channel subunit beta-1 (Neophocaena asiaeorientalis . . . ) | 100.0% |
| A0A2K5YZB1 | Sodium channel subunit beta-1 (Mandrillus leucophaeus) | 100.0% |
| A0A0D9QX16 | Sodium channel subunit beta-1 (Chlorocebus sabaeus) | 100.0% |
| A0A452G6B7 | Sodium channel subunit beta-1 (Capra hircus) | 100.0% |
| A0A6P3REC1 | Sodium channel subunit beta-1 (Pteropus vampyrus) | 100.0% |
| A0A484H424 | Sodium channel subunit beta-1 (Sousa chinensis) | 100.0% |
| A0A6J3QER2 | Sodium channel subunit beta-1 (Tursiops truncatus) | 100.0% |
| A0A3Q7MTY8 | Sodium channel subunit beta-1 (Callorhinus ursinus) | 100.0% |
| A0A340YF00 | Sodium channel subunit beta-1 (Lipotes vexillifer) | 100.0% |
| A0A7J8CLI4 | Sodium channel subunit beta-1 (Rousettus aegyptiacus) | 100.0% |
| A0A0A0MX70 | Sodium channel subunit beta-1 (Papio anubis) | 100.0% |
| A0A455AF03 | Sodium channel subunit beta-1 (Physeter macrocephalus) | 100.0% |
| M3WAE2 | Sodium channel subunit beta-1 (Felis catus) | 100.0% |
| A0A2K5NWE1 | Sodium channel subunit beta-1 (Cercocebus atys) | 100.0% |
| H0WMC9 | Sodium channel subunit beta-1 (Otolemur garnettii) | 100.0% |
| A0A2K5RB58 | Sodium channel subunit beta-1 (Cebus imitator) | 100.0% |
| A0A384DU30 | Sodium channel subunit beta-1 (Ursus maritimus) | 100.0% |
| F6Y804 | Sodium channel subunit beta-1 (Callithrix jacchus) | 100.0% |
| F6QID2 | Sodium channel subunit beta-1 (Macaca mulatta) | 100.0% |
| A0A2U3ZYX1 | Sodium channel subunit beta-1 (Odobenus rosmarus divergens) | 100.0% |
| A0A3Q7VQN4 | Sodium channel subunit beta-1 (Ursus arctos horribilis) | 100.0% |
| A0A2Y9L4L5 | Sodium channel subunit beta-1 (Enhydra lutris kenyoni) | 100.0% |
| A0A6J1Y6N4 | Sodium channel subunit beta-1 (Acinonyx jubatus) | 100.0% |
| A0A452G6A2 | Sodium channel subunit beta-1 (Capra hircus) | 100.0% |
| G5BXK3 | Sodium channel subunit beta-1 (Heterocephalus glaber) | 100.0% |
| A0A2K5JFG5 | Sodium channel subunit beta-1 (Colobus angolensis palliatus) | 100.0% |
| A0A2K6SIZ2 | Sodium channel subunit beta-1 (Saimiri boliviensis bolivien . . . ) | 100.0% |
| A0A2K5JFG1 | Sodium channel subunit beta-1 (Colobus angolensis palliatus) | 100.0% |
| A0A5J5MTP5 | Sodium channel subunit beta-1 (Muntiacus reevesi) | 100.0% |
| A0A6J0X7C7 | Sodium channel subunit beta-1 (Odocoileus virginianus texanus) | 100.0% |
| A0A2K5YZB4 | Sodium channel subunit beta-1 (Mandrillus leucophaeus) | 100.0% |
| A0A2U3X4F8 | Sodium channel subunit beta-1 (Odobenus rosmarus divergens) | 100.0% |
| F1PJY6 | Sodium channel subunit beta-1 (Canis lupus familiaris) | 100.0% |
| W5P3K3 | Sodium channel subunit beta-1 (Ovis aries) | 100.0% |
| A0A3Q7MTG1 | Sodium channel subunit beta-1 (Callorhinus ursinus) | 100.0% |
| A0A2U3XH47 | Sodium channel subunit beta-1 (Leptonychotes weddellii) | 100.0% |
| A0A2K6DUH1 | Sodium channel subunit beta-1 (Macaca nemestrina) | 100.0% |
| A0A2K6RI19 | Sodium channel subunit beta-1 (Rhinopithecus roxellana) | 100.0% |
| A0A2I3M1E6 | Sodium channel subunit beta-1 (Papio anubis) | 100.0% |
| A0A2K5JFF1 | Sodium channel subunit beta-1 (Colobus angolensis palliatus) | 100.0% |
| A0A7J8CLQ7 | Sodium channel subunit beta-1 (Rousettus aegyptiacus) | 100.0% |
| A0A7J7F8E4 | Sodium channel subunit beta-1 (Diceros bicornis minor) | 100.0% |
| A0A5F7ZEV3 | Sodium channel subunit beta-1 (Macaca mulatta) | 100.0% |
| A0A5E4AF02 | Peptidase S1 domain-containing protein (Marmota monax) | 100.0% |
| A0A5E4AEQ6 | Peptidase S1 domain-containing protein (Marmota monax) | 100.0% |
| L8Y2T7 | GRAM domain-containing protein 1A (Tupaia chinensis) | 100.0% |
| A0A1U7R697 | Sodium channel subunit beta-1 (Mesocricetus auratus) | 98.5% |
| F7CZ78 | Sodium channel subunit beta-1 (Equus caballus) | 98.5% |
| A0A1A6H394 | Sodium channel subunit beta-1 (Neotoma lepida) | 98.5% |
| G3IBD2 | GRAM domain-containing protein 1A (Cricetulus griseus) | 98.5% |
| A5A6L6 | Sodium channel subunit beta-1 (Pan troglodytes) | 98.5% |
| Q07699 | Sodium channel subunit beta-1 (Homo sapiens) | 98.5% |
| Q17QN4 | Sodium channel subunit beta-1 (Bos taurus) | 98.5% |
| A0A3Q1MVL1 | Sodium channel subunit beta-1 (Bos taurus) | 98.5% |
| M0QSP6 | Sodium channel subunit beta-1 (Bos mutus) | 98.5% |
| A0A2R9ALH1 | Sodium channel subunit beta-1 (Pan paniscus) | 98.5% |
| A0A2I2ZES7 | Sodium channel subunit beta-1 (Gorilla gorilla gorilla) | 98.5% |
| A0A2R9AET0 | Sodium channel subunit beta-1 (Pan paniscus) | 98.5% |
| H0WDA9 | Sodium channel subunit beta-1 (Cavia porcellus) | 98.5% |
| A0A4W2E9N6 | Sodium channel subunit beta-1 (Bos indicus × Bos taurus) | 98.5% |
| H2NYE4 | Sodium channel subunit beta-1 (Pongo abelii) | 98.5% |
| A0A1S3AS27 | Sodium channel subunit beta-1 (Erinaceus europaeus) | 98.5% |
| A0A6P3I605 | Sodium channel subunit beta-1 (Bison bison bison) | 98.5% |
| A0A6P5DCB6 | Sodium channel subunit beta-1 (Bos indicus) | 98.5% |
| A0A673V7Q0 | Sodium channel subunit beta-1 (Suricata suricatta) | 98.5% |
| I3LSX4 | Sodium channel subunit beta-1 (Sus scrofa) | 98.5% |
| A0A2I3TLR8 | Sodium channel subunit beta-1 (Pan troglodytes) | 98.5% |
| A0A287AGM2 | Sodium channel subunit beta-1 (Sus scrofa) | 98.5% |
| S9Y1X1 | Uncharacterized protein (Camelus ferus) | 98.5% |
| A0A0G2JXY6 | Sodium channel subunit beta-1 (Rattus norvegicus) | 100.0% |
| A0A3Q7UVI1 | Sodium channel subunit beta-1 (Ursus arctos horribilis) | 100.0% |
| A0A2K6DUJ3 | Sodium channel subunit beta-1 (Macaca nemestrina) | 100.0% |
| A0A2K6LH27 | Sodium channel subunit beta-1 (Rhinopithecus bieti) | 100.0% |
| A0A6J3FHZ6 | Sodium channel subunit beta-1 (Sapajus apella) | 100.0% |
| A0A6P6H3R2 | Sodium channel subunit beta-1 (Puma concolor) | 100.0% |
| A0A2K5V2A2 | Sodium channel subunit beta-1 (Macaca fascicularis) | 100.0% |
| A0A7J8CLB0 | Sodium channel subunit beta-1 (Rousettus aegyptiacus) | 100.0% |
| A0A455AQ41 | Sodium channel subunit beta-1 (Physeter macrocephalus) | 100.0% |
| A0A7F8QIS3 | Sodium channel subunit beta-1 (Leptonychotes weddellii) | 100.0% |
| A0A2K5NVZ3 | Sodium channel subunit beta-1 (Cercocebus atys) | 100.0% |
| A0A2Y9L668 | Sodium channel subunit beta-1 (Enhydra lutris kenyoni) | 100.0% |
| A0A3Q7UFE4 | Sodium channel subunit beta-1 (Vulpes vulpes) | 100.0% |
| A0A383YQP2 | Sodium channel subunit beta-1 (Balaenoptera acutorostrata s . . . ) | 100.0% |
| A0A2K6RI27 | Sodium channel subunit beta-1 (Rhinopithecus roxellana) | 100.0% |
| A0A2K5YZB5 | Sodium channel subunit beta-1 (Mandrillus leucophaeus) | 100.0% |
| A0A2K5NW07 | Sodium channel subunit beta-1 (Cercocebus atys) | 100.0% |
| A0A2Y9E6Y8 | Sodium channel subunit beta-1 (Trichechus manatus latirostris) | 96.9% |
| L5LT48 | Sodium channel subunit beta-1 (Myotis davidii) | 98.5% |
| A0A4X2MAM9 | Sodium channel subunit beta-1 (Vombatus ursinus) | 96.9% |
| A0A7J7SDQ2 | Sodium channel subunit beta-1 (Myotis myotis) | 98.5% |
| A0A6J2N7D5 | Sodium channel subunit beta-1 (Phyllostomus discolor) | 98.5% |
| A0A6P5JQ86 | Sodium channel subunit beta-1 (Phascolarctos cinereus) | 96.9% |
| S7P5D0 | Serine protease hepsin (Myotis brandtii) | 98.5% |
| A0A7J8CAY3 | Sodium channel subunit beta-1 (Molossus molossus) | 98.5% |
| A0A7J7V1I8 | Sodium channel subunit beta-1 (Pipistrellus kuhlii) | 98.5% |
| A0A6Q8PHF7 | Sodium channel subunit beta-1 (Homo sapiens) | 98.4% |
| A0A6I8PG08 | Sodium channel subunit beta-1 (Ornithorhynchus anatinus) | 98.4% |
| A0A1U7UN77 | Sodium channel subunit beta-1 (Carlito syrichta) | 98.4% |
| G3QQT7 | Sodium channel subunit beta-1 (Gorilla gorilla gorilla) | 98.4% |
| A0A1W2PR05 | Sodium channel subunit beta-1 (Homo sapiens) | 98.4% |
| A0A6Q8PGS1 | Sodium channel subunit beta-1 (Homo sapiens) | 98.4% |
| A0A2I2ZEH7 | Sodium channel subunit beta-1 (Gorilla gorilla gorilla) | 98.4% |
| A0A1W2PS68 | Sodium channel subunit beta-1 (Homo sapiens) | 98.4% |
| A0A6I9LE70 | Sodium channel subunit beta-1 (Peromyscus maniculatus bairdii) | 96.9% |
| A0A671EMW7 | Sodium channel subunit beta-1 (Rhinolophus ferrumequinum) | 95.4% |
| A0A6P5JJA7 | Sodium channel subunit beta-1 (Phascolarctos cinereus) | 96.9% |
| A0A1S3FYT3 | Sodium channel subunit beta-1 (Dipodomys ordii) | 96.9% |
| A0A6J3A145 | Sodium channel subunit beta-1 (Vicugna pacos) | 96.9% |
| F6X5N0 | Sodium channel subunit beta-1 (Monodelphis domestica) | 95.4% |
| G3SSP5 | Sodium channel subunit beta-1 (Loxodonta africana) | 95.5% |
| G3U3M7 | Sodium channel subunit beta-1 (Loxodonta africana) | 95.5% |
| A0A6I9JYT4 | Sodium channel subunit beta-1 (Chrysochloris asiatica) | 93.8% |
| A0A091EAT1 | Sodium channel subunit beta-1 (Fukomys damarensis) | 78.0% |
| A0A2Y9HY05 | Sodium channel subunit beta-1 (Neomonachus schauinslandi) | 92.2% |
| A0A5F8A1Z2 | Sodium channel subunit beta-1 (Macaca mulatta) | 98.2% |
| A0A5N4DV80 | Serine protease hepsin (Camelus dromedarius) | 78.1% |
| G1Q8T1 | Sodium channel subunit beta-1 (Myotis lucifugus) | 100.0% |
| A0A452I7Z8 | Sodium channel subunit beta-1 (Gopherus agassizii) | 67.2% |
| A0A4D9DHH1 | Sodium channel subunit beta-1 (Platysternon megacephalum) | 67.2% |
| M7B2K3 | GRAM domain-containing protein 1B (Chelonia mydas) | 65.6% |
| K7FJP5 | Sodium channel subunit beta-1 (Pelodiscus sinensis) | 64.1% |
| K7FJN6 | Sodium channel subunit beta-1 (Pelodiscus sinensis) | 64.1% |
| A0A670ILV9 | Sodium channel subunit beta-1 (Podarcis muralis) | 64.6% |
| A0A670IJL8 | Sodium channel subunit beta-1 (Podarcis muralis) | 64.6% |
| A0A5F4D4M7 | Sodium channel subunit beta-1 (Canis lupus familiaris) | 90.5% |
| A0A151NLD3 | Sodium channel subunit beta-1 (Alligator mississippiensis) | 62.5% |
| A0A3Q0FUE0 | Sodium channel subunit beta-1 (Alligator sinensis) | 62.5% |
| A0A2I3HWG7 | Sodium channel subunit beta-1 (Nomascus leucogenys) | 94.7% |
| A0A2I3GLW6 | Sodium channel subunit beta-1 (Nomascus leucogenys) | 94.7% |
| B4DI92 | Sodium channel subunit beta-1 (Homo sapiens) | 88.1% |
| G1RJA2 | Sodium channel subunit beta-1 (Nomascus leucogenys) | 94.6% |
| A0A6I9Y1F3 | Sodium channel subunit beta-1 (Thamnophis sirtalis) | 60.0% |
| A0A6J1VM60 | Sodium channel subunit beta-1 (Notechis scutatus) | 57.8% |
| A0A6P9CA31 | Sodium channel subunit beta-1 (Pantherophis guttatus) | 60.0% |
| A0A670ZDK5 | Sodium channel subunit beta-1 (Pseudonaja textilis) | 57.8% |
| A0A670ZC75 | Sodium channel subunit beta-1 (Pseudonaja textilis) | 57.8% |
| A0A6J0VAB9 | Sodium channel subunit beta-1 (Pogona vitticeps) | 58.7% |
| A0A670ZG95 | Sodium channel subunit beta-1 (Pseudonaja textilis) | 58.1% |
| H9GAJ2 | Sodium channel subunit beta-1 (Anolis carolinensis) | 54.7% |
| A0A3Q2TRR5 | Sodium channel subunit beta-1 (Gallus gallus) | 55.4% |
| A0A674ICU4 | Uncharacterized protein (Terrapene carolina triunguis) | 69.0% |
| A0A672UPE5 | Sodium voltage-gated channel beta subunit 1 (Strigops habroptila) | 52.3% |
| A0A674HND5 | Sodium channel subunit beta-1 (Taeniopygia guttata) | 50.8% |
| A0A674GBS6 | Sodium channel subunit beta-1 (Taeniopygia guttata) | 50.8% |
| A0A674HLS7 | Sodium channel subunit beta-1 (Taeniopygia guttata) | 50.8% |
| A0A6P7YXM3 | Sodium channel subunit beta-1 (Microcaecilia unicolor) | 50.8% |
| A0A1L8FHR9 | Sodium channel subunit beta-1 (Xenopus laevis) | 49.2% |
| A0A3P8VKV1 | Sodium voltage-gated channel beta subunit 1 (Cynoglossus | 54.7% |
| | semilaevis) | |
| A0A3P8VI91 | Sodium voltage-gated channel beta subunit 1 (Cynoglossus | 54.7% |
| | semilaevis) | |
| A0A7J6AFL4 | Ig-like domain-containing protein (Ameiurus melas) | 51.6% |
| A0A3P8VKV4 | Sodium voltage-gated channel beta subunit 1 (Cynoglossus | 54.7% |
| | semilaevis) | |
| A0A218U7M1 | Sodium channel subunit beta-1 (Lonchura striata domestica) | 50.8% |
| A0A6I8Q6I0 | Sodium channel subunit beta-1 (Xenopus tropicalis) | 49.2% |
| A0A6I8PU75 | Sodium channel subunit beta-1 (Xenopus tropicalis) | 49.2% |
| A0A4W5MWP1 | Sodium channel, voltage-gated, type I, beta a (Hucho hucho) | 52.5% |
| A0A4W5MZM4 | Sodium channel, voltage-gated, type I, beta a (Hucho hucho) | 52.5% |
| A0A5N5NCV9 | Ig-like domain-containing protein (Pangasianodon hypophthalmus) | 51.6% |
| A0A6P8NJH3 | Sodium channel subunit beta-1 (Geotrypetes seraphini) | 47.6% |
| A0A6P8P6N0 | Sodium channel subunit beta-1 (Geotrypetes seraphini) | 47.6% |
| A0A674EFD1 | Sodium channel subunit beta-1-like (Salmo trutta) | 50.8% |
| A0A674EF26 | Sodium channel subunit beta-1-like (Salmo trutta) | 50.8% |
| A0A6G1QXG5 | Sodium channel subunit beta-1 (Channa argus) | 51.6% |
| H3A6Y1 | Sodium channel subunit beta-1 (Latimeria chalumnae) | 46.9% |
| W5MC27 | Sodium channel subunit beta-1 (Lepisosteus oculatus) | 48.4% |
| W5MC40 | Sodium channel subunit beta-1 (Lepisosteus oculatus) | 48.4% |
| A0A498NKG6 | Sodium channel subunit beta-1-like protein (Labeo rohita) | 48.5% |
| A0A3B1J362 | Ig-like domain-containing protein (Astyanax mexicanus) | 48.4% |
| A0A3B1K0P4 | Ig-like domain-containing protein (Astyanax mexicanus) | 48.4% |
| A0A3P9DJY9 | Sodium voltage-gated channel beta subunit 1 (Maylandia zebra) | 48.4% |
| A0A3Q3RSS0 | Sodium channel, voltage-gated, type I, beta b | 51.6% |
| | (Mastacembelus armatus) | |
| A0A3P9DKZ9 | Sodium voltage-gated channel beta subunit 1 (Maylandia zebra) | 48.4% |
| A0A498MNP3 | Sodium channel subunit beta-1-like protein (Labeo rohita) | 46.9% |
| A0A673H978 | Sodium channel, voltage-gated, type I, beta b | 46.9% |
| | (Sinocyclocheilus rhinocerous) | |
| A0A673HAT7 | Sodium channel, voltage-gated, type I, beta b | 46.9% |
| | (Sinocyclocheilus rhinocerous) | |
| A0A1L8FPK6 | Sodium channel subunit beta-1 (Xenopus laevis) | 47.6% |
| A0A060YS67 | Ig-like domain-containing protein (Oncorhynchus mykiss) | 50.8% |
| A0A6P6LKC5 | sodium channel subunit beta-1-like (Carassius auratus) | 47.0% |
| A0A1S3RV12 | sodium channel subunit beta-1-like (Salmo salar) | 50.8% |
| A0A674CF94 | Sodium channel subunit beta-1-like (Salmo trutta) | 50.8% |
| A0A060VSY7 | Ig-like domain-containing protein (Oncorhynchus mykiss) | 50.8% |
| A0A484CJ90 | Ig-like domain-containing protein (Perca flavescens) | 50.0% |
| A0A674CG83 | Sodium channel subunit beta-1-like (Salmo trutta) | 50.8% |
| A0A6P8W1P3 | sodium channel subunit beta-1-like (Gymnodraco acuticeps) | 48.4% |
| A0A0P7XNM1 | Sodium channel beta-1 subunit-like (Scleropages formosus) | 48.4% |
| A0A673H7U8 | Sodium channel, voltage-gated, type I, beta b | 46.9% |
| | (Sinocyclocheilus rhinocerous) | |
| A0A4W5KMS4 | Sodium channel subunit beta-1 (Hucho hucho) | 50.8% |
| A0A4W5L189 | Sodium channel, voltage-gated, type I, beta a (Hucho hucho) | 50.8% |
| A0A1S3P0F3 | sodium channel subunit beta-1-like isoform X2 (Salmo salar) | 49.2% |
| A0A6A5F1F9 | Ig-like domain-containing protein (Perca fluviatilis) | 50.0% |
| A0A1S3P0D1 | sodium channel subunit beta-1-like isoform X1 (Salmo salar) | 49.2% |
| G3ND81 | Sodium channel, voltage-gated, type I, beta b (Gasterosteus aculeatus) | 46.9% |
| A0A4W6E6X8 | Sodium voltage-gated channel beta subunit 1 (Lates calcarifer) | 48.4% |
| A0A6P6LXD1 | sodium channel subunit beta-1-like isoform X3 (Carassius auratus) | 45.3% |
| A0A4W5KY24 | Ig-like domain-containing protein (Hucho hucho) | 48.4% |
| A0A6P6LUG4 | sodium channel subunit beta-1-like isoform X1 (Carassius auratus) | 45.3% |
| A0A1S3M0Y3 | sodium channel subunit beta-1-like isoform X2 (Salmo salar) | 48.4% |
| A0A673FJ06 | Sodium channel subunit beta-1-like (Sinocyclocheilus rhinocerous) | 46.9% |
| A0A3P9A632 | Sodium channel subunit beta-1 (Esox lucius) | 49.2% |
| A0A672KNU6 | Sodium channel subunit beta-1-like (Sinocyclocheilus grahami) | 46.9% |
| A0A7N6BWF0 | Sodium channel, voltage-gated, type I, beta b (Anabas testudineus) | 48.4% |
| A0A3P8Z616 | Sodium channel subunit beta-1-like (Esox lucius) | 48.4% |
| A0A674CT99 | Sodium channel subunit beta-1-like (Salmo trutta) | 48.4% |
| A0A1S3M1D8 | sodium channel subunit beta-1-like isoform X1 (Salmo salar) | 48.4% |
| A0A672ZD25 | Ig-like domain-containing protein (Sphaeramia orbic | |

### Exemplary Strategies to Increase the Stability of the SCN1B Mimetic Pareptides

In some embodiments, the SCN1B mimetic peptides have one or more modifications that increase peptide stability. In some embodiments, the SCN1B mimetic peptides have one or more modifications that increase peptide stability in vivo. The one or more modifications that can modify, such as increase, peptide stability can be in the N-terminus, the C-terminus, between the N- and C-terminus, or any combination thereof. In some embodiments, the one or more modifications that increase the stability of the SCN1B mimetic peptide is a chemical modification to one or more amino acid residues. Exemplary chemical modifications include, but are not limited to, glycosylation (e.g., N-glycosylation, O-glycoslylation, glypiation, C-glycosylation, and phosphoglycosylation), amidation, and acetylation, S-nitrosylation, lipidation (C-terminal glycosyl phosphatidylinositol (GPI) anchors), N-terminal myristoylation, S-myristoylation, and S-prenylation)), PEGylation (attachment of Polyethylene glycol (PEG)), and any combination thereof. Without being bound by theory, modifications such as acetylation or glycosylation of the N-terminus or amidation of the C-terminus can prevent binding of exogenous proteases, protecting the peptide from enzymatic degradation, thereby increasing the stability of the SCN1B mimetic peptide. Polyethylene glycol (PEG) is a polyether that has high water solubility, low toxicity, and high mobility in solution. PEG is a large molecule, on which peptides can be attached via their C- or N-terminus.

In some embodiments, the SCN1B mimetic peptide or one or more residue(s) thereof are attached, conjugated, or otherwise associated with one or more macromolecules (e.g., albumin and/or the like) to increase the stability of the SCN1B mimetic peptide. Without being bound by theory the macromolecule, such as albumin or other blood plasma proteins, protect against peptide degradation. Macromolecules can be conjugated to the SCN1B mimetic peptides using any suitable technique generally known in the art, such as chemical conjugation based methods and techniques.

In some embodiments, the one or more modifications that modify, e.g., increase, stability of the SCN1B mimetic peptide is a chemical modification to one or more amino acid residues.

In some embodiments, the stability of the SCN1B mimetic peptide is increased with one or more conservative amino acid substitutions, such as substitutions of L with D amino acids, substitutions with non-natural amino acids.

In some embodiments, the SCN1B mimetic peptide can be stabilized and/or protected from degradation by incorporation into a delivery vehicle such as any of those described elsewhere herein and others generally known in the art that will be appreciated in view of the description provided herein. In some embodiments, the SCN1B mimetic peptides have one or more modifications that facilitate and/or enhance incorporation and/or uptake of the SCN1B mimetic peptides into a delivery vehicle and/or cells. In some embodiments, the SCN1B mimetic peptides are esterified at one or more residues such as at the N- and/or C-terminus. Without being bound by theory, esterification of the SCN1B mimetic peptides can facilitate and/or enhance loading and/or retention of the esterified SCN1B mimetic peptides in an exosome and/or facilitate uptake into cells.

In some embodiments, the SCN1B mimetic peptides are included in a pharmaceutical formulation that is adapted for extended release and/or otherwise formulated to prevent degradation of the SCN1B peptides, and/or increase SCN1B mimetic peptide stability and/or half-life. Exemplary pharmaceutical formulations and dosages are described in greater detail elsewhere herein.

In some embodiments, the SNC1B mimetic peptides are included in a delivery device that is configured to protect (e.g., prevent degradation) as well as deliver the SCN1B mimetic peptides. In some embodiments, the delivery devices is composed or manufactured by non-biodegradable material(s) (such as ceramics, metals or metal alloys, and/or the like) which can protect the SCN1B mimetic peptides. In some embodiments, the devices can have a mechanical or osmotic pump comprising a reservoir capable of holding sufficient peptide for a few days, a catheter that is inserted subcutaneously for delivery, a battery, and a processor that adjusts the frequency and amount of peptide delivered can be used to control delivery of the peptides.

Nanoparticles made from the organic (e.g., dendrimers, polymersomes, PLGA) or inorganic (e.g., gold, silica) materials conjugated with the peptides can be used to control delivery of peptides. Other nanoparticles and delivery vehicles that may be suitable for increasing the stability and/or half-life of the SCN1B mimetic peptides are described in greater detail elsewhere herein.

In some embodiments, the SCN1B mimetic peptides are cyclized. Without being bound by theory, a cyclic SCN1B mimetic peptide can have improved stability and thus activity in vivo. In some embodiments, the SCN1B mimetic peptides are modified by including cysteines at the N and C terminal ends of the SCN1B mimetic peptides where the SCN1B mimetic peptide does not contain an N or C terminal cysteine. The peptides can be cyclized by disulfide bonding between the N- and C-terminal cysteine residues.

### Other Peptide Modifications

In some embodiments, the peptides of the present invention can include one or more modifications that can provide additional functionalities. Exemplary modifications include, but are not limited to, signal peptides for secretion, targeting or other trafficking domain. Generally, trafficking domains are elements capable of directing transport or location, referred to herein as trafficking domains. Trafficking includes, without limitation, a) secretion, b) nuclear localization, c) nuclear export, d) transport to an organelle, e) lysosomal transport, f) injection (such as from a pathogen into a target cell, g) binding to a cell surface receptor, h) binding to a soluble receptor, i) binding to a soluble target, and j) binding to an immobilized target.

In some embodiments the trafficking domain is or contains s a secretion signal peptide.

A trafficking domain can include one or more amino acid sequence capable of binding to another molecule (e.g., a receptor, antigen, antibody) or a cell (such as a cell surface receptor).

A trafficking domain may be a component of a specific binding pair (SBP). SBPs include without limitation, antigen-antibody binding, ligand-receptor binding, and more generally protein-protein interactions such as avidin-biotin. In such embodiments, the trafficking domain can be used to target a protein or polypeptide to a cellular receptor or cell type. In certain embodiments, the targeting is inducible, for example by providing a protein or polypeptide of the invention and separately providing at or for a selected time an agent that binds to or forms a complex with the protein or polypeptide of the invention and directs the complex to a ligand, receptor, or cell type.

In some embodiments, the invention includes an SCN1B mimetic peptides of the present invention includes a secretion signal peptide. In an embodiment the secretion signal peptide provides export from a bacterial cell. In an embodiment, the secretion signal peptide provides export from a eukaryotic cell. In an embodiment, the secretion signal peptide provides export from a mammalian cell. In an embodiment, SCN1B mimetic peptides of the present invention can be collected and purified from culture media. In certain embodiments, the trafficking domain comprises an N-terminal secretion signal peptide. Exemplary secretion signal peptides are generally known in the art and include, but are not limited to, a Tat peptide (S/T-R—R-X-F-L-K, SEQ ID NO: 43) (Palmer et al., The twin-arginine translocation (Tat) protein export pathway. Nat Rev Microbiol. 2012; 10:483-496. doi: 10.1038/nrmicro2814), a Sec signal peptide, and/or the like. See also e.g., Klatt and Konthur et al., Microb Cell Fact. 2012; 11:97, Yarimizu et al., Microbial Cell Factories volume 14, Article number: 20 (2015), al., Peng et Front. Bioeng. Biotechnol., 11 Jun. 2019, https://doi.org/10.3389/fbioe.2019.00139, particularly at Table 1, Aza et al., Cellular and Molecular Life Sciences volume 78, pages 3691-3707 (2021), which are incorporated by reference herein and can be adapted for use with the present invention.

### SCN1B Mimetic Peptide Encoding Polynucleotides

Described in several exemplary embodiments herein are nucleic acid constructs and polynucleotides that encode one or more of the SCN1B memetic peptides. In some embodiments, the nucleic acid construct or polypeptide includes one or more regulatory elements coupled to the polynucleotide(s) encoding the one or more SCN1B memetic peptides. In some embodiments, the nucleic acid construct and/or encoding polynucleotide is DNA, RNA, DNA: RNA hybrid or other nucleic acid.

In certain example embodiments, the nucleic acid construct and/or encoding polynucleotide further comprises a polynucleotide encoding a reporter polypeptide, wherein the polynucleotide encoding the reporter polypeptide is operably coupled to the SCN1B mimetic peptide encoding polynucleotide, optionally via linker encoding polynucleotide or direct fusion, thereby encoding a SCN1B mimetic peptide operably coupled to a reporter protein. In certain example embodiments, the reporter polypeptide is an optically active polypeptide, optionally a fluorescent polypeptide. In certain example embodiments, the reporter polypeptide is configured to produce a signal or a loss of a signal, optionally an optical signal, upon expression. Reporter polypeptides and other configurations of the nucleic acid construct are described in greater detail elsewhere herein.

### Codon Optimization

As described elsewhere herein, the polynucleotide encoding one or more embodiments of the SCN1B mimetic polypeptides and other polypeptides of the present disclosure described herein can be codon optimized. In some embodiments, one or more polynucleotides contained in a vector (“vector polynucleotides”) described herein that are in addition to an optionally codon optimized polynucleotide encoding embodiments of the genetic modifying system described herein can be codon optimized. Vectors are described in greater detail elsewhere herein. In general, codon optimization refers to a process of modifying a nucleic acid sequence for enhanced expression in the host cells of interest by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.orjp/codon/and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, PA), are also available. In some embodiments, one or more codons (e.g., 1, 2, 3, 4, 5, 10, 15, 20, 25, 50, or more, or all codons) in a sequence encoding a SCN1B mimetic peptide of the present invention described elsewhere herein corresponds to the most frequently used codon for a particular amino acid. As to codon usage in yeast, reference is made to the online Yeast Genome database available at www.yeastgenome.org/community/codon_usage.shtml, or Codon selection in yeast, Bennetzen and Hall, J Biol Chem. 1982 Mar. 25;257 (6): 3026-31. As to codon usage in plants including algae, reference is made to Codon usage in higher plants, green algae, and cyanobacteria, Campbell and Gowri, Plant Physiol. 1990 January; 92 (1): 1-11.; as well as Codon usage in plant genes, Murray et al, Nucleic Acids Res. 1989 Jan. 25;17 (2): 477-98; or Selection on the codon bias of chloroplast and cyanelle genes in different plant and algal lineages, Morton B R, J Mol Evol. 1998 April;46 (4): 449-59.

The SCN1B mimetic peptide encoding polynucleotide or other polynucleotide (such as a vector polynucleotide) can be codon optimized for expression in a specific cell-type, tissue type, organ type, and/or subject type, such as a mammalian cell, optionally a human cell. In some embodiments, a codon optimized sequence is a sequence optimized for expression in a eukaryote, e.g., bovines (i.e., being optimized for expression in a mammalian cell, optionally a human cell), or for another eukaryote, such as another animal (e.g., a bovine, equine, canine, feline, ovine, and/or the like). Such codon optimized sequences are within the ambit of the ordinary skilled artisan in view of the description herein. In some embodiments, the polynucleotide is codon optimized for a specific cell type. Such cell types can include, but are not limited to, epithelial cells (including skin cells, cells lining the gastrointestinal tract, cells lining other hollow organs), nerve cells (nerves, brain cells, spinal column cells, nerve support cells (e.g., astrocytes, glial cells, Schwann cells etc.), muscle cells (e.g., cardiac muscle, smooth muscle cells, and skeletal muscle cells), connective tissue cells (fat and other soft tissue padding cells, bone cells, tendon cells, cartilage cells), blood cells, stem cells (including embryonic stem cells, primordial germ cells, primordial germ cell like cells, pluripotent stem cells, totipotent stem cells, blastocysts, etc.) and other progenitor cells, immune system cells, germ cells, and combinations thereof. Such codon optimized sequences are within the ambit of the ordinary skilled artisan in view of the description herein. In some embodiments, the polynucleotide is codon optimized for a specific tissue type. Such tissue types can include, but are not limited to, muscle tissue, connective tissue, connective tissue, nervous tissue, and epithelial tissue. Such codon optimized sequences are within the ambit of the ordinary skilled artisan in view of the description herein. In some embodiments, the polynucleotide is codon optimized for a specific organ. Such organs include, but are not limited to, muscles, skin, intestines, liver, spleen, brain, lungs, stomach, heart, kidneys, gallbladder, pancreas, bladder, thyroid, bone, blood vessels, blood, and combinations thereof. Such codon optimized sequences are within the ambit of the ordinary skilled artisan in view of the description herein.

In some embodiments, a vector polynucleotide is codon optimized for expression in particular cells, such as prokaryotic or eukaryotic cells. The eukaryotic cells may be those of or derived from a particular organism, such as a mammal, including but not limited to human, or non-human eukaryote or animal or mammal as discussed herein, e.g., a bovine, ovine, canine, feline, Delivery of Polynucleotides and Polypeptides,

The SCN1B mimetic peptides and/or encoding polynucleotides can be delivered to a cell or cell population by any suitable method, technique, and/or system.

### Physical Delivery

In some embodiments, the SCN1B mimetic peptides and/or encoding polynucleotides and/or nucleic acid constructs can be introduced to cells by physical delivery methods. Examples of physical methods include microinjection, electroporation, and hydrodynamic delivery. Both nucleic acids (DNA and/or RNA) and proteins may be delivered using such methods. For example, SCN1B peptide may be prepared in vitro, isolated, (refolded and purified if needed), and introduced to cells by a physical delivery method or technique.

### Microinjection

Microinjection of the SCN1B mimetic peptides and/or encoding polynucleotides and/or nucleic acid constructs, or other delivery vehicles containing the same directly to cells can achieve high efficiency, e.g., above 90% or about 100%. In some embodiments, microinjection may be performed using a microscope and a needle (e.g., with 0.5-5.0 μm in diameter) to pierce a cell membrane and deliver the cargo directly to a target site within the cell. Microinjection may be used for in vitro and ex vivo delivery.

Plasmids or other nucleic acid constructs containing SCN1B mimetic peptide encoding polynucleotides and/or nucleic acid constructs, coding sequences for Cas or other genetic modifying system effector proteins and/or any associated polynucleotides (e.g., guide RNAs, mRNAs, and/or guide RNAs), may be microinjected. In some cases, microinjection may be used i) to deliver DNA directly to a cell nucleus, and/or ii) to deliver mRNA (e.g., in vitro transcribed) to a cell nucleus or cytoplasm. In certain examples, microinjection may be used to delivery sgRNA directly to the nucleus and Cas or other effector protein-encoding mRNA to the cytoplasm, e.g., facilitating translation and shuttling of Cas or other effector protein to the cell nucleus. The genetic modifying systems can be used to modify a cell, such as a genome of a cell, to contain a SCN1B mimetic peptides encoding polynucleotides and/or nucleic acid construct.

Microinjection may be used to generate genetically modified animals or cells such as those described elsewhere herein, such as those containing and/or expressing the SCN1B mimetic peptides and/or encoding polynucleotides and/or nucleic acid constructs of the present disclosure. For example, gene modification systems or components thereof may be injected into zygotes, blastomeres, blastocysts, embryonic stem cells, pluripotent stem cells, induced pluripotent stem cells, primordial germ cells, primordial germ cell like-cells, and/or the like to allow for gene medication, such as germline modification.

### Electroporation

In some embodiments, the SCN1B mimetic peptides and/or encoding polynucleotides and/or nucleic acid constructs, genetic modifying systems, and/or delivery vehicles containing the same may be delivered by electroporation. Electroporation may use pulsed high-voltage electrical currents to transiently open nanometer-sized pores within the cellular membrane of cells suspended in buffer, allowing for components with hydrodynamic diameters of tens of nanometers to flow into the cell. In some cases, electroporation may be used on various cell types and efficiently transfer cargo (e.g., the SCN1B mimetic peptides and/or encoding polynucleotides and/or nucleic acid constructs, and/or delivery vehicles containing the same) into cells. Electroporation may be used for in vitro and ex vivo delivery.

Electroporation may also be used to deliver the cargo (e.g., the SCN1B mimetic peptides and/or encoding polynucleotides and/or nucleic acid constructs, and/or delivery vehicles containing the same) to into the nuclei of mammalian cells by applying specific voltage and reagents, e.g., by nucleofection. Such approaches include those described in Wu Y, et al. (2015). Cell Res 25:67-79; Ye L, et al. (2014). Proc Natl Acad Sci USA 111:9591-6; Choi P S, Meyerson M. (2014). Nat Commun 5:3728; Wang J, Quake S R. (2014). Proc Natl Acad Sci 111:13157-62. Electroporation may also be used to deliver the cargo in vivo, e.g., with methods described in Zuckermann M, et al. (2015). Nat Commun 6:7391.

### Hydrodynamic Delivery

Hydrodynamic delivery may also be used for delivering the SCN1B mimetic peptides and/or encoding polynucleotides and/or nucleic acid constructs, genetic modifying systems, and/or delivery vehicles containing the same, e.g., for in vivo delivery. In some examples, hydrodynamic delivery may be performed by rapidly pushing a large volume (8-10% body weight) solution containing the gene modification system into the bloodstream of a subject (e.g., a bovine). As blood is incompressible, the large bolus of liquid may result in an increase in hydrodynamic pressure that temporarily enhances permeability into endothelial and parenchymal cells, allowing for cargo not normally capable of crossing a cellular membrane to pass into cells. This approach may be used for delivering naked DNA plasmids and proteins.

### Transfection

The SCN1B mimetic peptides and/or encoding polynucleotides and/or nucleic acid constructs, and/or delivery vehicles containing the same, genetic modifying system, and/or other polypeptides and/or polynucleotides described herein, may be introduced to cells by transfection methods for introducing nucleic acids into cells. Examples of transfection methods include calcium phosphate-mediated transfection, cationic transfection, liposome transfection, dendrimer transfection, heat shock transfection, magnetofection, lipofection, impalefection, optical transfection, proprietary agent-enhanced uptake of nucleic acid. Nucleic acids and vectors and vector systems that can encode a genetic modifying system and/or components thereof are described in greater detail else wherein herein. Transfection has been used to deliver nucleic acid constructs to bovine cells. See e.g., Tajik et al., Iran J Vet Res. 2017 Spring; 18 (2): 113-118; Jafarnejad et al., S African J Anim Sci, Vol. 48 No. 1 (2018) DOI: 10.4314/sajas.v48i1.13; Duarte et al., Anim Biotechnol. 2020 Dec. 30;1-11. doi: 10.1080/10495398.2020.1862137; and Osorio Gene. 2017 Aug. 30; 626:200-208, which are incorporated by reference as if expressed in their entireties herein and can be adapted for use with the present disclosure.

### Transduction

The SCN1B mimetic peptides and/or encoding polynucleotides and/or nucleic acid constructs, and/or delivery vehicles containing the same, genetic modifying system, and/or other polypeptides and/or polynucleotides described herein, can be introduced to cells by transduction by a viral, pseudoviral, and/or virus like particle. Methods of packaging the SCN1B mimetic peptides and/or encoding polynucleotides and/or nucleic acid constructs, and/or delivery vehicles containing the same, genetic modifying system, and/or other polypeptides and/or polynucleotides described herein in viral particles can be accomplished using any suitable viral vector or vector systems. Such viral vector and vector systems are described in greater detail elsewhere herein. As used in this context herein “transduction” refers to the process by which foreign nucleic acids and/or proteins are introduced to a cell (prokaryote or eukaryote) by a viral, pseudoviral, and/or virus like particle. After packaging in a viral, pseudoviral, and/or virus like particle, the viral particles can be exposed to cells (e.g., in vitro, ex vivo, or in vivo) where the viral, pseudoviral, and/or virus like particle infects the cell and delivers the cargo (e.g., SCN1B mimetic peptides and/or encoding polynucleotides and/or nucleic acid constructs, and/or delivery vehicles containing the same, genetic modifying system, and/or other polypeptides and/or polynucleotides described herein) to the cell via transduction. Viral, pseudoviral, and/or virus like particles can be optionally concentrated prior to exposure to target cells. In some embodiments, the virus titer of a composition containing viral and/or pseudoviral particles can be obtained and a specific titer be used to transduce cells. Viral vectors and systems and generation of viral (or pseudoviral, and/or virus like particle) delivery particles is described in greater detail elsewhere herein.

### Biolisties

The SCN1B mimetic peptides and/or encoding polynucleotides and/or nucleic acid constructs, and/or delivery vehicles containing the same, genetic modifying system, and/or other polypeptides and/or polynucleotides described herein can be introduced to cells using a biolistic method or technique. The term of art “biolistic”, as used herein refers to the delivery of nucleic acids to cells by high-speed particle bombardment. In some embodiments, the genetic modifying systems and/or components thereof can be attached, associated with, or otherwise coupled to particles, which than can be delivered to the cell via a gene-gun (see e.g., Liang et al. 2018. Nat. Protocol. 13:413-430; Svitashev et al. 2016. Nat. Comm. 7:13274; Ortega-Escalante et al., 2019. Plant. J. 97:661-672). In some embodiments, the particles can be gold, tungsten, palladium, rhodium, platinum, or iridium particles.

### Implantable Devices

In some embodiments, the delivery system can include an implantable device that incorporates or is coated with a genetic modifying systems and/or components thereof described herein. Various implantable devices are described in the art, and include any device, graft, or other composition that can be implanted into a subject, such as a human or other non-human animal.

### Delivery Vehicles

Polynucleotides and/or polypeptides of the present disclosure, such as a SCN1B mimetic peptides and/or encoding polynucleotides and/or nucleic acid constructs, and/or delivery vehicles containing the same, genetic modifying system, and/or other polypeptides and/or polynucleotides described herein, can be delivered (e.g., to a target cell to be modified) via one or more delivery vehicles. The delivery vehicles can deliver a cargo, such as a polynucleotide or polypeptide of the present disclosure (such as a SCN1B mimetic peptides and/or encoding polynucleotides and/or nucleic acid constructs, and/or delivery vehicles containing the same, genetic modifying system, and/or other polypeptides and/or polynucleotides described herein), into cells, tissues, organs, or organisms (e.g., animals or plants). In some embodiments, delivery vehicles are sued to deliver a cargo, such as a polynucleotide or polypeptide of the present disclosure to a target human or other non-human animal cell. The cargos may be packaged, carried, or otherwise associated with the delivery vehicles. The delivery vehicles may be selected based on the types of cargo to be delivered, and/or the delivery is in vitro and/or in vivo. Examples of delivery vehicles include vectors, viruses (e.g., virus particles, pseudoviral particles, or virus like particles), non-viral vehicles (e.g., exosomes, liposomes, etc.), and other delivery reagents described herein and those appreciated by one of ordinary skill in the art in view of the present disclosure.

The delivery vehicles described herein can have a greatest dimension or greatest average dimension (e.g., diameter or greatest average diameter) of less than 100 microns (μm). In some embodiments, the delivery vehicles have a greatest dimension or greatest average dimension of less than 10 μm. In some embodiments, the delivery vehicles may have a greatest dimension or greatest average dimension of less than 2000 nanometers (nm). In some embodiments, the delivery vehicles may have a greatest dimension or greatest average dimension of less than 1000 nanometers (nm). In some embodiments, the delivery vehicles may have a greatest dimension or greatest average dimension (e.g., diameter or average diameter) of less than 900 nm, less than 800 nm, less than 700 nm, less than 600 nm, less than 500 nm, less than 400 nm, less than 300 nm, less than 200 nm, less than 150 nm, or less than 100 nm, less than 50 nm. In some embodiments, the delivery vehicles may have a greatest dimension or greatest average dimension ranging between 25 nm and 200 nm.

### Particles

In some embodiments, the delivery vehicles may be or comprise particles. For example, the delivery vehicle may be or comprise nanoparticles (e.g., particles with a greatest dimension or greatest average dimension (e.g., diameter or greatest average diameter) no greater than 1000 nm. The particles may be provided in different forms, e.g., as solid particles (e.g., metal such as silver, gold, iron, titanium), non-metal, lipid-based solids, polymers), suspensions of particles, or combinations thereof. Metal, dielectric, and semiconductor particles may be prepared, as well as hybrid structures (e.g., core-shell particles).

Nanoparticles may also be used to deliver the compositions and systems to cells, as described in US20130185823, WO2008042156, and WO2015089419. In general, a “nanoparticle” refers to any particle having a diameter of less than 1000 nm. In certain embodiments, nanoparticles of the invention have a greatest dimension or greatest average dimension (e.g., diameter or average diameter) of 500 nm or less. In other embodiments, nanoparticles of the invention have a greatest dimension or greatest average dimension ranging between 25 nm and 200 nm. In other embodiments, nanoparticles of the invention have a greatest dimension or greatest average dimension of 100 nm or less. In other embodiments, nanoparticles of the invention have a greatest dimension or greatest average dimensions ranging between 35 nm and 60 nm. It will be appreciated that reference made herein to particles or nanoparticles can be interchangeable, where appropriate. Nanoparticles made of semiconducting material may also be labeled quantum dots if they are small enough (typically sub 10 nm) that quantization of electronic energy levels occurs. Such nanoscale particles are used in biomedical applications as drug carriers or imaging agents and may be adapted for similar purposes in the present invention. Semi-solid and soft nanoparticles have been manufactured and are within the scope of the present invention. Nanoparticles with one half hydrophilic and the other half hydrophobic are termed Janus particles and are particularly effective for stabilizing emulsions. They can self-assemble at water/oil interfaces and act as solid surfactants.

In some embodiments, the nanoparticles are Poly (lactic co-glycolic acids) (PLGA) nanoparticles. See e.g., Infate, J. C., Front. Neurosci., 19 Jul. 2018, https://doi.org/10.3389/fnins.2018.00484; Li, et al. (2001). PEGylated PLGA nanoparticles as protein carriers: synthesis, preparation and biodistribution in rats. Journal of controlled release: official journal of the Controlled Release Society. 71. 203-11. 10.1016/S0168-3659 (01) 00218-8; and Mohammadi-Samani and Taghipour. 2-15. Pharmaceutical Development Tech. 20 (4): 385-393).

Particle characterization (including e.g., characterizing morphology, dimension, etc.) is done using a variety of different techniques. Common techniques are electron microscopy (TEM, SEM), atomic force microscopy (AFM), dynamic light scattering (DLS), X-ray photoelectron spectroscopy (XPS), powder X-ray diffraction (XRD), Fourier transform infrared spectroscopy (FTIR), matrix-assisted laser desorption/ionization time-of-flight mass spectrometry (MALDI-TOF), ultraviolet-visible spectroscopy, dual polarization interferometry and nuclear magnetic resonance (NMR). Characterization (dimension measurements) may be made as to native particles (i.e., preloading) or after loading of the cargo (e.g., one or more components of a genetic modifying system (e.g., a CRISPR-Cas system or component(s) thereof) and can include additional carriers and/or excipients) to provide particles of an optimal size for delivery for any in vitro, ex vivo and/or in vivo application of the present disclosure. In some embodiments, particle dimension (e.g., diameter) characterization is based on measurements using dynamic laser scattering (DLS). See also e.g., U.S. Pat. Nos. 8,709,843; 6,007,845; 5,855,913; 5,985,309; 5,543,158; and Dahlman et al. Nature Nanotechnology (2014), doi: 10.1038/nnano.2014.84, describes particles, methods of making and using them, and measurements thereof, which can be adapted for use with the present disclosure.

### Vectors and Vector Systems

In some embodiments the delivery vehicle is a vector or vector system or particle, such as a virus or viral like particle, produced from such a vector or vector system. As such, also provided herein are vectors that can contain one or more of the SCN1B mimetic peptide encoding polynucleotides described herein. In certain embodiments, the vector can contain one or more polynucleotides encoding one or more elements of a genetic modifying system described herein. The vectors can be useful in producing bacterial, fungal, yeast, plant cells, animal cells, and transgenic animals that can express one or more components of the genetic modifying system described herein, and as such, contain a genetic modification, such as one or more of the SCN1B mimetic peptide encoding polynucleotides described herein or be rendered capable of producing particles (e.g., viral or viral like particles) that can be used to deliver a genetic modifying system and/or a one or more of the SCN1B mimetic peptide encoding polynucleotides described herein described herein to a cell, such as a human or non-human animal cell.

Within the scope of this disclosure are vectors containing one or more of the polynucleotide sequences described herein, such as those relevant to introducing one or more SCN1B mimetic polypeptide encoding polynucleotide. One or more of the polynucleotides that are part of a genetic modifying system can be included in a vector or vector system. The vectors and/or vector systems can be used, for example, to express one or more of the polynucleotides in a cell, such as a producer cell, to produce a genetic modifying system containing virus particles described elsewhere herein. Other uses for the vectors and vector systems described herein are also within the scope of this disclosure. In general, and throughout this specification, the term “vector” refers to a tool that allows or facilitates the transfer of an entity from one environment to another. In some contexts which will be appreciated by those of ordinary skill in the art, “vector” can be a term of art to refer to a nucleic acid molecule capable of transporting another nucleic acid to which it has been linked. A vector can be a replicon, such as a plasmid, phage, or cosmid, into which another DNA segment may be inserted so as to bring about the replication of the inserted segment. Generally, a vector is capable of replication when associated with the proper control elements.

Vectors include, but are not limited to, nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g., retroviruses, replication defective retroviruses, adenoviruses, replication defective adenoviruses, and adeno-associated viruses (AAVs)). Viral vectors also include polynucleotides carried by a virus for transfection into a host cell. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Common expression vectors of utility in recombinant DNA techniques are often in the form of plasmids.

Recombinant expression vectors can be composed of a nucleic acid (e.g., a polynucleotide) of the invention in a form suitable for expression of the nucleic acid in a host cell, which means that the recombinant expression vectors include one or more regulatory elements, which can be selected on the basis of the host cells to be used for expression, that is operatively-linked to the nucleic acid sequence to be expressed. Within a recombinant expression vector, “operably linked” and “operatively-linked” are used interchangeably herein and mean that the nucleotide sequence of interest is linked to the regulatory element(s) in a manner that allows for expression of the nucleotide sequence (e.g., in an in vitro transcription/translation system or in a host cell when the vector is introduced into the host cell). Advantageous vectors include lentiviruses and adeno-associated viruses, and types of such vectors can also be selected for targeting particular types of cells. These and other embodiments of the vectors and vector systems are described elsewhere herein.

In some embodiments, the vector can be a bicistronic vector. In some embodiments, a bicistronic vector can be used for one or more elements of the genetic modifying system described herein. In some embodiments, expression of elements of the genetic modifying system described herein can be driven by the CBh promoter or other ubiquitous promoter. Where the element of the genetic modifying system is an RNA, its expression can be driven by a Pol III promoter, such as a U6 promoter. In some embodiments, the two are combined.

### Cell-based Vector Amplification and Expression

Vectors may be introduced and propagated in a prokaryotic cell or eukaryotic cell. In some embodiments, a prokaryote is used to amplify copies of a vector to be introduced into a eukaryotic cell or as an intermediate vector in the production of a vector to be introduced into a eukaryotic cell (e.g., amplifying a plasmid as part of a viral vector packaging system). The vectors can be viral-based or non-viral based. In some embodiments, a prokaryote is used to amplify copies of a vector and express one or more nucleic acids, such as to provide a source of one or more proteins for delivery to a host cell or host organism.

Vectors can be designed for expression of one or more elements of the genetic modifying system described herein (e.g., nucleic acid transcripts, proteins, enzymes, and combinations thereof) in a suitable host cell. In some embodiments, the suitable host cell is a prokaryotic cell. Suitable host cells include, but are not limited to, bacterial cells, yeast cells, insect cells, and mammalian cells. In some embodiments, the suitable host cell is a eukaryotic cell. In some embodiments the host cell is a cell to be modified by a genetic modifying system. In some embodiments the host cell is a producer cell capable of producing particles (e.g., virus particles, virus like particles, exosomes, and/or the like) that can be used to deliver a genetic modifying system or component thereof to a cell.

In some embodiments, the suitable host cell is a suitable bacterial cell. Suitable bacterial cells include but are not limited to bacterial cells from the bacteria of the species Escherichia coli. Many suitable strains of E. coli are known in the art for expression of vectors. These include, but are not limited to Pir1, Stb12, Stb13, Stb14, TOP10, XL1 Blue, and XL10 Gold. In some embodiments, the host cell is a suitable insect cell. Suitable insect cells include those from Spodoptera frugiperda. Suitable strains of S. frugiperda cells include, but are not limited to, Sf9 and Sf21. In some embodiments, the host cell is a suitable yeast cell. In some embodiments, the yeast cell can be from Saccharomyces cerevisiae. In some embodiments, the host cell is a suitable mammalian cell. Many types of mammalian cells have been developed to express vectors. Suitable mammalian cells include, but are not limited to, HEK293, Chinese Hamster Ovary Cells (CHOs), mouse myeloma cells, HeLa, U2OS, A549, HT1080, CAD, P19, NIH 3T3, L929, N2a, MCF-7, Y79, SO-Rb50, HepG G2, DIKX-X11, J558L, Baby hamster kidney cells (BHK), and chicken embryo fibroblasts (CEFs). Suitable host cells are discussed further in Goeddel, GENE EXPRESSION TECHNOLOGY: METHODS IN ENZYMOLOGY 185, Academic Press, San Diego, Calif. (1990). In some embodiments, the suitable host cell is a bovine cell, including but not limited to, bovine embryonic stem cells, bovine induced pluripotent stem cells, bovine blastocyst cells, bovine spermatogonia stem cells, bovine oogonial cells, bovine primordial germ cells, bovine primordial germ cell like cells, bovine totipotent cells, or other bovine cell described elsewhere herein.

In some embodiments, the vector can be a yeast expression vector. Examples of vectors for expression in yeast Saccharomyces cerevisiae include pYepSecl (Baldari, et al., 1987. EMBO J. 6:229-234), pMFa (Kuijan and Herskowitz, 1982. Cell 30:933-943), pJRY88 (Schultz et al., 1987. Gene 54:113-123), pYES2 (Invitrogen Corporation, San Diego, Calif.), and picZ (InVitrogen Corp, San Diego, Calif.). As used herein, a “yeast expression vector” refers to a nucleic acid that contains one or more sequences encoding an RNA and/or polypeptide and may further contain any desired elements that control the expression of the nucleic acid(s), as well as any elements that enable the replication and maintenance of the expression vector inside the yeast cell. Many suitable yeast expression vectors and features thereof are known in the art; for example, various vectors and techniques are illustrated in in Yeast Protocols, 2^{nd} edition, Xiao, W., ed. (Humana Press, New York, 2007) and Buckholz, R. G. and Gleeson, M. A. (1991) Biotechnology (NY) 9 (11): 1067-72. Yeast vectors can contain, without limitation, a centromeric (CEN) sequence, an autonomous replication sequence (ARS), a promoter, such as an RNA Polymerase III promoter, operably linked to a sequence or gene of interest, a terminator such as an RNA polymerase III terminator, an origin of replication, and a marker gene (e.g., auxotrophic, antibiotic, or other selectable markers). Examples of expression vectors for use in yeast may include plasmids, yeast artificial chromosomes, 2u plasmids, yeast integrative plasmids, yeast replicative plasmids, shuttle vectors, and episomal plasmids.

In some embodiments, the vector is a baculovirus vector or expression vector and can be suitable for expression of polynucleotides and/or proteins in insect cells. In some embodiments, the suitable host cell is an insect cell. Baculovirus vectors available for expression of proteins in cultured insect cells (e.g., SF9 cells) include the pAc series (Smith, et al., 1983. Mol. Cell. Biol. 3:2156-2165) and the pVL series (Lucklow and Summers, 1989. Virology 170:31-39). rAAV (recombinant Adeno-associated viral) vectors are preferably produced in insect cells, e.g., Spodoptera frugiperda Sf9 insect cells, grown in serum-free suspension culture. Serum-free insect cells can be purchased from commercial vendors, e.g., Sigma Aldrich (EX-CELL 405).

In some embodiments, the vector is a mammalian expression vector. In some embodiments, the mammalian expression vector is capable of expressing one or more polynucleotides and/or polypeptides in a mammalian cell. Examples of mammalian expression vectors include, but are not limited to, pCDM8 (Seed, 1987. Nature 329:840) and pMT2PC (Kaufman, et al., 1987. EMBO J. 6:187-195). The mammalian expression vector can include one or more suitable regulatory elements capable of controlling expression of the one or more polynucleotides and/or proteins in the mammalian cell. For example, commonly used promoters are derived from polyoma, adenovirus 2, cytomegalovirus, simian virus 40, and others disclosed herein and known in the art. More detail on suitable regulatory elements is provided elsewhere herein.

For other suitable expression vectors and vector systems for both prokaryotic and eukaryotic cells see, e.g., Chapters 16 and 17 of Sambrook, et al., MOLECULAR CLONING: A LABORATORY MANUAL. 2^{nd} ed., Cold Spring Harbor Laboratory, Cold Spring Harbor Laboratory Press, Cold Spring Harbor, N.Y., 1989.

In some embodiments, the vector can be a fusion vector or fusion expression vector. In some embodiments, fusion vectors add a number of amino acids to a protein encoded therein, such as to the amino terminus, carboxy terminus, or both of a recombinant protein. Such fusion vectors can serve one or more purposes, such as: (i) to increase expression of recombinant protein; (ii) to increase the solubility of the recombinant protein; and (iii) to aid in the purification of the recombinant protein by acting as a ligand in affinity purification. In some embodiments, expression of polynucleotides (such as non-coding polynucleotides) and proteins in prokaryotes can be carried out in Escherichia coli with vectors containing constitutive or inducible promoters directing the expression of either fusion or non-fusion polynucleotides and/or proteins. In some embodiments, the fusion expression vector can include a proteolytic cleavage site, which can be introduced at the junction of the fusion vector backbone or other fusion moiety and the recombinant polynucleotide or protein to enable separation of the recombinant polynucleotide or protein from the fusion vector backbone or other fusion moiety subsequent to purification of the fusion polynucleotide or protein. Such enzymes, and their cognate recognition sequences, include Factor Xa, thrombin and enterokinase. Example fusion expression vectors include pGEX (Pharmacia Biotech Inc; Smith and Johnson, 1988. Gene 67:31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein. Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

In some embodiments, one or more vectors driving expression of one or more SCN1B mimetic polypeptide encoding polynucleotides are introduced into a host cell such that expression of the one or more SCN1B mimetic polypeptide encoding polynucleotides occurs within the host cell.

In some embodiments, one or more vectors driving expression of one or more elements of a genetic modifying system described herein are introduced into a host cell such that expression of the elements of the delivery system described herein direct formation of a genetic modifying system complex (e.g., a CRISPR-Cas complex) at one or more target sites at on a target polynucleotide, such as in a target cell or target cell genome. For example, a CRISPR-Cas effector protein describe herein and a nucleic acid component (e.g., a guide polynucleotide) can each be operably linked to separate regulatory elements on separate vectors. RNA(s) of different elements of a genetic modifying (e.g., CRISPR-Cas) system can be delivered to an animal, plant, microorganism or cell thereof to produce an animal (e.g., a mammal, such as a bovine)), that constitutively, inducibly, or conditionally expresses different elements of the genetic modifying (e.g., CRISPR-Cas) system described herein that incorporates one or more elements of the genetic modifying system (e.g., a CRISPR-Cas system) described herein or contains one or more cells that incorporates and/or expresses one or more elements of the genetic modifying (e.g., CRISPR-Cas) system described herein.

### Cell-Free Vector and Polynucleotide Expression

In some embodiments, the polynucleotide encoding one or more features of the genetic modifying system or other polynucleotide described herein can be expressed from a vector or suitable polynucleotide in a cell-free in vitro system. In other words, the polynucleotide can be transcribed and optionally translated in vitro. In vitro transcription/translation systems and appropriate vectors are generally known in the art and commercially available. Generally, in vitro transcription and in vitro translation systems replicate the processes of RNA and protein synthesis, respectively, outside of the cellular environment. Vectors and suitable polynucleotides for in vitro transcription can include T7, SP6, T3, promoter regulatory sequences that can be recognized and acted upon by an appropriate polymerase to transcribe the polynucleotide or vector.

In vitro translation can be stand-alone (e.g., translation of a purified polyribonucleotide) or linked/coupled to transcription. In some embodiments, the cell-free (or in vitro) translation system can include extracts from rabbit reticulocytes, wheat germ, and/or E. coli. The extracts can include various macromolecular components that are needed for translation of exogenous RNA (e.g., 70S or 80S ribosomes, tRNAs, aminoacyl-tRNA, synthetases, initiation, elongation factors, termination factors, etc.). Other components can be included or added during the translation reaction, including but not limited to, amino acids, energy sources (ATP, GTP), energy regenerating systems (creatine phosphate and creatine phosphokinase (eukaryotic systems)) (phosphoenol pyruvate and pyruvate kinase for bacterial systems), and other co-factors (Mg²⁻, K+, etc.). As previously mentioned, in vitro translation can be based on RNA or DNA starting material. Some translation systems can utilize an RNA template as starting material (e.g., reticulocyte lysates and wheat germ extracts). Some translation systems can utilize a DNA template as a starting material (e.g., E coli-based systems). In these systems transcription and translation are coupled and DNA is first transcribed into RNA, which is subsequently translated. Suitable standard and coupled cell-free translation systems are generally known in the art and are commercially available.

### Vector Features

The vectors can include additional features that can confer one or more functionalities to the vector, the polynucleotide to be delivered, a virus or other particle (e.g., viral like particle or exosome) produced there from, or polypeptide expressed thereof. Such features include, but are not limited to, regulatory elements, selectable markers, molecular identifiers (e.g., molecular barcodes), stabilizing elements, and the like. It will be appreciated by those skilled in the art that the design of the expression vector and additional features included can depend on such factors as the choice of the host cell to be transformed, the level of expression desired, etc.

### Regulatory Elements

In certain embodiments, the polynucleotides and/or vectors thereof described herein (such as SCN1B mimetic polypeptide encoding polynucleotides described herein) can include one or more regulatory elements that can be operatively linked to the polynucleotide. The term “regulatory element” is intended to include promoters, enhancers, internal ribosomal entry sites (IRES), other expression control elements (e.g., transcription termination signals, such as polyadenylation signals and poly-U sequences) and cellular localization signals (e.g., nuclear localization or export signals). Such regulatory elements are described, for example, in Goeddel, GENE EXPRESSION TECHNOLOGY: METHODS IN ENZYMOLOGY 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter can direct expression primarily in a desired tissue of interest, such as muscle, neuron, bone, skin, blood, specific organs (e.g., liver, pancreas), or particular cell types (e.g., lymphocytes). Regulatory elements may also direct expression in a temporal-dependent manner, such as in a cell-cycle dependent or developmental stage-dependent manner, which may or may not also be tissue or cell-type specific. In some embodiments, a vector comprises one or more pol III promoter (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) (see, e.g., Boshart et al, Cell, 41:521-530 (1985)), the SV40 promoter, the dihydrofolate reductase promoter, the β-actin promoter, the phosphoglycerol kinase (PGK) promoter, and the EF1α promoter. Also encompassed by the term “regulatory element” are enhancer elements, such as WPRE; CMV enhancers; the R-U5′ segment in LTR of HTLV-I (Mol. Cell. Biol., Vol. 8 (1), p. 466-472, 1988); SV40 enhancer; and the intron sequence between exons 2 and 3 of rabbit β-globin (Proc. Natl. Acad. Sci. USA., Vol. 78 (3), p. 1527-31, 1981). Exemplary promoters also include bovine U6 (bU6) and bovine 7SK (b7SK), and other bovine PolII promoters (see e.g., Lambeth et al., Anim Genet. 2006 August;37 (4): 369-72), bovine papillomavirus-1 promoters (BPV-1) (Linz and Baker. J Virol. 1988 August;62 (8): 2537-43. doi: 10.1128/JVI.62.8.2537-2543.1988), the bovine SIX1 gene promoter (see e.g., Wei et al. Scientific Reports volume 7, Article number: 12599 (2017)), bovine growth hormone promoter (see e.g., Jiang et al., Nuc Acid Prot Syn Mol Gen. 1999. 274 (12): 7893-7900), bovine pyruvate carboxylase (see e.g., Hazelton et al. J. Dairy Sci. 91:91-99), a bidirectional promoter (see e.g., Meersserman et al. DNA Research, Volume 24, Issue 3, June 2017, Pages 221-233), a bovine Akt3 promoter (see e.g., Farmanullah et al. Journal of Genetic Engineering and Biotechnology (2021) 19:164), bovine alpha-lactalbumin promoter (see e.g., FEBS Lett. 1991 Jun. 17;284 (1): 19-22), bovine beta-casein promoter (see e.g., Cerdan et al., Mol Reprod Dev. 1998 March;49 (3): 236-45), any combination thereof . . .

In some embodiments, the regulatory sequence can be a regulatory sequence described in U.S. Pat. No. 7,776,321, U.S. Pat. Pub. No. 2011/0027239, or International Patent Publication No. WO 2011/028929, the contents of which are incorporated by reference herein in their entireties. In some embodiments, the vector can contain a minimal promoter. In some embodiments, the minimal promoter is the Mecp2 promoter, tRNA promoter, or U6. In a further embodiment, the minimal promoter is tissue specific. In some embodiments, the length of the vector polynucleotide the minimal promoters and polynucleotide sequences is less than 4.4Kb.

To express a polynucleotide, the vector can include one or more transcriptional and/or translational initiation regulatory sequences, e.g., promoters, that direct the transcription of the gene and/or translation of the encoded protein in a cell. In some embodiments a constitutive promoter may be employed. Suitable constitutive promoters for mammalian cells are generally known in the art and include, but are not limited to SV40, CAG, CMV, EF-1α, β-actin, RSV, and PGK. Suitable constitutive promoters for bacterial cells, yeast cells, and fungal cells are generally known in the art, such as a T-7 promoter for bacterial expression and an alcohol dehydrogenase promoter for expression in yeast.

In some embodiments, the regulatory element can be a regulated promoter. As used herein, “regulated promoter” refers to promoters that direct gene expression not constitutively, but in a temporally- and/or spatially-regulated manner, and includes tissue-specific, tissue-preferred and inducible promoters. Regulated promoters include conditional promoters and inducible promoters. In some embodiments, conditional promoters can be employed to direct expression of a polynucleotide in a specific cell type, under certain environmental conditions, and/or during a specific state of development. Suitable tissue specific promoters can include, but are not limited to, liver specific promoters (e.g. APOA2, SERPIN Al (hAAT), CYP3A4, and MIR122), pancreatic cell promoters (e.g. INS, IRS2, Pdx1, Alx3, Ppy), cardiac specific promoters (e.g. Myh6 (alpha MHC), MYL2 (MLC-2v), TNI3 (cTnl), NPPA (ANF), Slc8al (Ncx1)), central nervous system cell promoters (SYN1, GFAP, INA, NES, MOBP, MBP, TH, FOXA2 (HNF3 beta)), skin cell specific promoters (e.g. FLG, K14, TGM3), immune cell specific promoters, (e.g. ITGAM, CD43 promoter, CD14 promoter, CD45 promoter, CD68 promoter), urogenital cell specific promoters (e.g. Pbsn, Upk2, Sbp, Fer114), endothelial cell specific promoters (e.g. ENG), pluripotent and embryonic germ layer cell specific promoters (e.g. Oct4, NANOG, Synthetic Oct4, T brachyury, NES, SOX17, FOXA2, MIR122), and muscle cell specific promoter (e.g. myostatin, Desmin). Other tissue and/or cell specific promoters are generally known in the art and are within the scope of this disclosure.

Inducible/conditional promoters can be positively inducible/conditional promoters (e.g. a promoter that activates transcription of the polynucleotide upon appropriate interaction with an activated activator, or an inducer (compound, environmental condition, or other stimulus) or a negative/conditional inducible promoter (e.g., a promoter that is repressed (e.g., bound by a repressor) until the repressor condition of the promotor is removed (e.g., inducer binds a repressor bound to the promoter stimulating release of the promoter by the repressor or removal of a chemical repressor from the promoter environment). The inducer can be a compound, environmental condition, or other stimulus. Thus, inducible/conditional promoters can be responsive to any suitable stimuli such as chemical, biological, or other molecular agents, temperature, light, and/or pH. Suitable inducible/conditional promoters include, but are not limited to, Tet-On, Tet-Off, Lac promoter, pBad, AlcA, LexA, Hsp70 promoter, Hsp90 promoter, pDawn, XVE/OlexA, GVG, and pOp/LhGR.

Examples of promoters that are inducible and that can allow for spatiotemporal control of gene editing or gene expression may use a form of energy. The form of energy may include, but is not limited to, sound energy, electromagnetic radiation, chemical energy and/or thermal energy. Examples of inducible systems include tetracycline inducible promoters (Tet-On or Tet-Off), small molecule two-hybrid transcription activations systems (FKBP, ABA, etc.), or light inducible systems (Phytochrome, LOV domains, or cryptochrome), such as a Light Inducible Transcriptional Effector (LITE) that direct changes in transcriptional activity in a sequence-specific manner. The components of a light inducible system may include one or more SCN1B mimetic peptides described herein, a light-responsive cytochrome heterodimer (e.g., from Arabidopsis thaliana), and a transcriptional activation/repression domain. In some embodiments, the vector can include one or more of the inducible DNA binding proteins provided in International Patent Publication No. WO 2014/018423 and U.S. Patent Publication Nos., 2015/0291966, 2017/0166903, 2019/0203212, which describe e.g., embodiments of inducible DNA binding proteins and methods of use and can be adapted for use with the present invention.

In some embodiments, transient or inducible expression can be achieved by including, for example, chemical-regulated promotors, i.e., whereby the application of an exogenous chemical induces gene expression. Modulation of gene expression can also be obtained by including a chemical-repressible promoter, where application of the chemical represses gene expression. Chemical-inducible promoters include, but are not limited to, the maize In2-2 promoter, activated by benzene sulfonamide herbicide safeners (De Veylder et al., (1997) Plant Cell Physiol 38:568-77), the maize GST promoter (GST-11-27, WO93/01294), activated by hydrophobic electrophilic compounds used as pre-emergent herbicides, and the tobacco PR-1 a promoter (Ono et al., (2004) Biosci Biotechnol Biochem 68:803-7) activated by salicylic acid. Promoters that are regulated by antibiotics, such as tetracycline-inducible and tetracycline-repressible promoters (Gatz et al., (1991) Mol Gen Genet 227:229-37; U.S. Pat. Nos. 5,814,618 and 5,789,156) can also be used herein.

In some embodiments where multiple elements are to be expressed from the same vector or within the same vector system, different promoters or regulatory elements can be used for each element to be expressed to avoid or limit loss of expression due to competition between promoters and/or other regulatory elements.

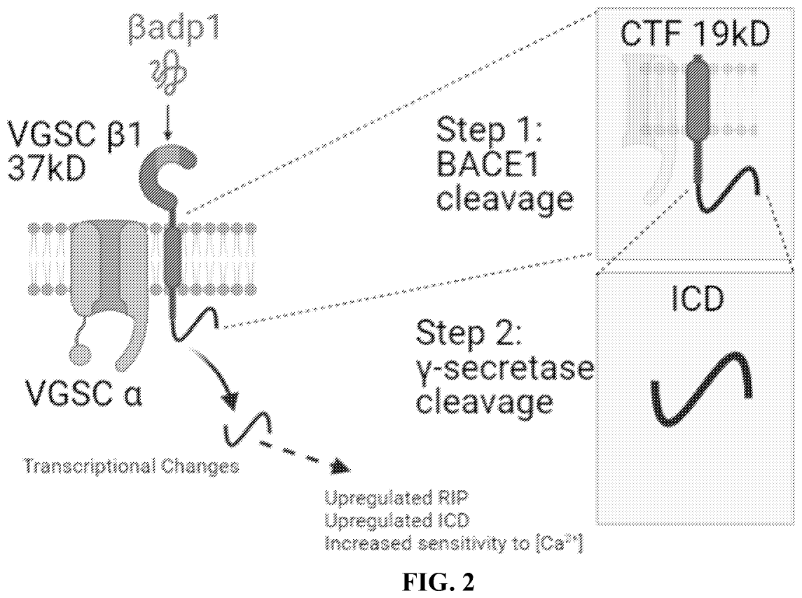

In some embodiments, the polynucleotide, vector or system thereof can include one or more elements capable of translocating and/or expressing a polynucleotide to/in a specific cell component or organelle. Such organelles can include, but are not limited to, nucleus, ribosome, endoplasmic reticulum, Golgi apparatus, chloroplast, mitochondria, vacuole, lysosome, cytoskeleton, plasma membrane, cell wall, peroxisome, centrioles, etc. Such regulatory elements can include, but are not limited to, nuclear localization signals (examples of which are described in greater detail elsewhere herein), any such as those that are annotated in the LocSigDB database (see e.g., genome.unmc.edu/LocSigDB/and Negi et al., 2015. Database. 2015: bav003; doi: 10.1093/database/bav003), nuclear export signals (e.g., LXXXLXXLXL (SEQ ID NO: 29) and others described elsewhere herein), endoplasmic reticulum localization/retention signals (e.g., KDEL (SEQ ID NO: 30), KDXX (SEQ ID NO: 31), KKXX (SEQ ID NO: 32), KXX (SEQ ID NO: 33), and others described elsewhere herein; and see e.g., Liu et al. 2007 Mol. Biol. Cell. 18 (3): 1073-1082 and Gorleku et al., 2011. J. Biol. Chem. 286:39573-39584), mitochondria targeting signals (see e.g., Chin, R. M., et al, 2018, Cell Reports. 22:2818-2826, particularly at FIG. 2; Doyle et al. 2013. PLOS ONE 8, e67938; Funes et al. 2002. J. Biol. Chem. 277:6051-6058; Matouschek et al. 1997. PNAS USA 85:2091-2095; Oca-Cossio et al., 2003. 165:707-720; Waltner et al., 1996. J. Biol. Chem. 271:21226-21230; Wilcox et al., 2005. PNAS USA 102:15435-15440; Galanis et al., 1991. FEBS Lett 282:425-430), and peroxisome targeting signals (e.g. (S/A/C)-(K/R/H)-(L/A) (SEQ ID NO: 34), SLK (SEQ ID NO: 35), (R/K)-(L/V/I)-XXXXX-(H/Q)-(L/A/F) (SEQ ID NO: 36)). Suitable protein targeting motifs can also be designed or identified using any suitable database or prediction tool, including but not limited to Minimotif Miner (minimotifminer.org, mitominer.mrc-mbu.cam.ac.uk/release-4.0/embodiment.do?name=Protein % 20MTS), LocDB (see above), PTSs predictor, TargetP-2.0 www.cbs.dtu.dk/services/TargetP/), ChloroP (www.cbs.dtu.dk/services/ChloroP/); NetNES (www.cbs.dtu.dk/services/NetNES/), Predotar (urgi.versailles.inra.fr/predotar/), and SignalP (www.cbs.dtu.dk/services/SignalP/).

### Selectable Markers and Tags

One or more of the polynucleotides described herein, such as those of or encoding a SCN1B mimetic polypeptide and/or other exogenous gene can be operably linked, fused to, or otherwise modified to include a polynucleotide that encodes or is a selectable marker or tag, which can be a polynucleotide or polypeptide. In some embodiments, the polypeptide encoding a polypeptide selectable marker is incorporated in the genetic modifying system polynucleotide or other polynucleotide of the present disclosure such that the selectable marker polypeptide, when translated, is inserted between two amino acids between the N- and C-terminus of the genetic modifying system polypeptide (or other polypeptide of the present disclosure) or at the N- and/or C-terminus of the genetic modifying system polypeptide (or other polypeptide of the present disclosure). In some embodiments, the selectable marker or tag is a polynucleotide barcode or unique molecular identifier (UMI).

It will be appreciated that the polynucleotide encoding such selectable markers or tags can be incorporated into a polynucleotide encoding one or more components of the genetic modifying system (or other polynucleotide) described herein in an appropriate manner to allow expression of the selectable marker or tag. Such techniques and methods are described elsewhere herein and will be instantly appreciated by one of ordinary skill in the art in view of this disclosure. Many such selectable markers and tags are generally known in the art and are intended to be within the scope of this disclosure.

Suitable selectable markers and tags include, but are not limited to, affinity tags, such as chitin binding protein (CBP), maltose binding protein (MBP), glutathione-S-transferase (GST), poly(His) tag; solubilization tags such as thioredoxin (TRX) and poly (NANP), MBP, and GST; chromatography tags such as those consisting of polyanionic amino acids, such as FLAG-tag; epitope tags such as V5-tag, Myc-tag, HA-tag and NE-tag; protein tags that can allow specific enzymatic modification (such as biotinylation by biotin ligase) or chemical modification (such as reaction with FLASH-EDT2 for fluorescence imaging), DNA and/or RNA segments that contain restriction enzyme or other enzyme cleavage sites; DNA segments that encode products that provide resistance against otherwise toxic compounds including antibiotics, such as, spectinomycin, ampicillin, kanamycin, tetracycline, Basta, neomycin phosphotransferase II (NEO), hygromycin phosphotransferase (HPT)) and the like; DNA and/or RNA segments that encode products that are otherwise lacking in the recipient cell (e.g., tRNA genes, auxotrophic markers); DNA and/or RNA segments that encode products which can be readily identified (e.g., phenotypic markers such as β-galactosidase, GUS; fluorescent proteins such as green fluorescent protein (GFP), cyan (CFP), yellow (YFP), red (RFP), luciferase, and cell surface proteins); polynucleotides that can generate one or more new primer sites for PCR (e.g., the juxtaposition of two DNA sequences not previously juxtaposed), DNA sequences not acted upon or acted upon by a restriction endonuclease or other DNA modifying enzyme, chemical, etc.; epitope tags (e.g. GFP, FLAG- and His-tags), and, DNA sequences that make a molecular barcode or unique molecular identifier (UMI), DNA sequences required for a specific modification (e.g., methylation) that allows its identification. Other suitable markers will be appreciated by those of skill in the art.

Selectable markers and tags can be operably linked to one or more components of the genetic modifying system (or other polypeptide) described herein via suitable linker, such as a glycine or glycine serine linkers as short as GS or GG up to (GGGGG) 3 (SEQ ID NO: 44) or (GGGGS) 3 (SEQ ID NO: 45). Other suitable linkers are described elsewhere herein.

### Targeting Moieties

The vector or vector system (or other polynucleotide) can include one or more polynucleotides that are or encode one or more targeting moieties. In some embodiments, the targeting moiety encoding polynucleotides can be included in the vector or vector system, such as a viral vector system, such that they are expressed within and/or on the virus particle(s) produced such that the virus particles can be targeted to specific cells, tissues, organs, etc. In some embodiments, the targeting moiety encoding polynucleotides can be included in the vector or vector system such that the genetic modifying system polynucleotide(s) and/or products expressed therefrom include the targeting moiety and can be targeted to specific cells, tissues, organs, etc. In some embodiments, such as non-viral carriers, the targeting moiety can be attached to the carrier (e.g., polymer, lipid, inorganic molecule etc.) and can be capable of targeting the carrier and any attached or associated genetic modifying system polynucleotide(s) to specific cells, tissues, organs, etc. In some embodiments, the targeting moieties can target integrins on cell surfaces. Optionally, the binding affinity of the targeting moiety is in the range of 1 nM to 1 μM.

Exemplary targeting moieties that can be included are described elsewhere herein. See description related to “Targeted Delivery” and/or “Responsive Delivery” herein.

### Codon Optimization

As described elsewhere herein, the polynucleotide encoding one or more embodiments of the SCN1B mimetic polypeptides or other polypeptides (such as those to be delivered to a target cell) of the present disclosure described herein can be codon optimized. In some embodiments, one or more polynucleotides contained in a vector (“vector polynucleotides”) described herein that are in addition to an optionally codon optimized polynucleotide encoding embodiments of the genetic modifying system described herein can be codon optimized. In general, codon optimization refers to a process of modifying a nucleic acid sequence for enhanced expression in the host cells of interest by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.orjp/codon/and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, PA), are also available. In some embodiments, one or more codons (e.g., 1, 2, 3, 4, 5, 10, 15, 20, 25, 50, or more, or all codons) in a sequence encoding a SCN1B mimetic peptide corresponds to the most frequently used codon for a particular amino acid. As to codon usage in yeast, reference is made to the Yeast online Genome database available at www.yeastgenome.org/community/codon_usage.shtml, or Codon selection in yeast, Bennetzen and Hall, J Biol Chem. 1982 Mar. 25;257 (6): 3026-31. As to codon usage in plants including algae, reference is made to Codon usage in higher plants, green algae, and cyanobacteria, Campbell and Gowri, Plant Physiol. 1990 January; 92 (1): 1-11.; as well as Codon usage in plant genes, Murray et al, Nucleic Acids Res. 1989 Jan. 25; 17 (2): 477-98; or Selection on the codon bias of chloroplast and cyanelle genes in different plant and algal lineages, Morton B R, J Mol Evol. 1998 April;46 (4): 449-59.

The vector polynucleotide can be codon optimized for expression in a specific cell-type, tissue type, organ type, and/or subject type, such as a human or other non-human animal cell. In some embodiments, a codon optimized sequence is a sequence optimized for expression in a eukaryote, e.g., human (i.e., being optimized for expression in a or human or human cell), or for another eukaryote, such as another animal (e.g., an ovine, bovine, feline, canine, equine, avian, and/or the like). Such codon optimized sequences are within the ambit of the ordinary skilled artisan in view of the description herein. In some embodiments, the polynucleotide is codon optimized for a specific cell type. Such cell types can include, but are not limited to, epithelial cells (including skin cells, cells lining the gastrointestinal tract, cells lining other hollow organs), nerve cells (nerves, brain cells, spinal column cells, nerve support cells (e.g. astrocytes, glial cells, Schwann cells etc.), muscle cells (e.g. cardiac muscle, smooth muscle cells, and skeletal muscle cells), connective tissue cells (fat and other soft tissue padding cells, bone cells, tendon cells, cartilage cells), blood cells, stem cells (including embryonic stem cells, primordial germ cells, primordial germ cell like cells, pluripotent stem cells, totipotent stem cells, blastocysts, etc.) and other progenitor cells, immune system cells, germ cells, and combinations thereof. Such codon optimized sequences are within the ambit of the ordinary skilled artisan in view of the description herein. In some embodiments, the polynucleotide is codon optimized for a specific tissue type. Such tissue types can include, but are not limited to, muscle tissue, connective tissue, connective tissue, nervous tissue, and epithelial tissue. Such codon optimized sequences are within the ambit of the ordinary skilled artisan in view of the description herein. In some embodiments, the polynucleotide is codon optimized for a specific organ. Such organs include, but are not limited to, muscles, skin, intestines, liver, spleen, brain, lungs, stomach, heart, kidneys, gallbladder, pancreas, bladder, thyroid, bone, blood vessels, blood, and combinations thereof. Such codon optimized sequences are within the ambit of the ordinary skilled artisan in view of the description herein.

In some embodiments, a vector polynucleotide is codon optimized for expression in particular cells, such as prokaryotic or eukaryotic cells. The eukaryotic cells may be those of or derived from a particular organism, such as a mammal, including but not limited to human, or non-human eukaryote or animal or mammal as discussed herein, e.g., a bovine, ovine, camelid, and/or the like.

### Vector Construction

The vectors described herein can be constructed using any suitable process or technique. In some embodiments, one or more suitable recombination and/or cloning methods or techniques can be used to the vector(s) described herein. Suitable recombination and/or cloning techniques and/or methods can include, but not limited to, those described in U.S. Patent Publication No. US 2004/0171156 A1. Other suitable methods and techniques are described elsewhere herein.

Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989). Any of the techniques and/or methods can be used and/or adapted for constructing an AAV or other vectors described herein, nAAV vectors are discussed elsewhere herein.

In some embodiments, a vector comprises one or more insertion sites, such as a restriction endonuclease recognition sequence (also referred to as a “cloning site”). In some embodiments, one or more insertion sites (e.g., about or more than about 1, 2, 3, 4, 5, 6, 7, 8, 9, 10, or more insertion sites) are located upstream and/or downstream of one or more sequence elements of one or more vectors. When multiple different guide polynucleotides are used, a single expression construct may be used to target nucleic acid-targeting activity to multiple different, corresponding target sequences within a cell. For example, a single vector may comprise about or more than about 1, 2, 3, 4, 5, 6, 7, 8, 9, 10, 15, 20, or more guide s polynucleotides. In some embodiments, about or more than about 1, 2, 3, 4, 5, 6, 7, 8, 9, 10, or more such guide-polynucleotide-containing vectors may be provided, and optionally delivered to a cell.

Delivery vehicles, vectors, particles, nanoparticles, formulations and components thereof for expression of one or more elements of a genetic modifying system or other polynucleotides described herein are as used in the foregoing documents, such as International Patent Publication No. WO 2014/093622 and are discussed in greater detail herein.

### Viral Vectors

In some embodiments, the vector is a viral vector. The term of art “viral vector” and as used herein in this context refers to polynucleotide based vectors that contain one or more elements from or based upon one or more elements of a virus that can be capable of expressing and packaging a polynucleotide, such as a genetic modifying system polynucleotide of the present invention, into a virus particle and producing said virus particle when used alone or with one or more other viral vectors (such as in a viral vector system). Viral vectors and systems thereof can be used for producing viral particles for delivery of and/or expression of one or more components of the genetic modifying system described herein. The viral vector can be part of a viral vector system involving multiple vectors. In some embodiments, systems incorporating multiple viral vectors can increase the safety of these systems. Suitable viral vectors can include retroviral-based vectors, lentiviral-based vectors, adenoviral-based vectors, adeno associated vectors, helper-dependent adenoviral (HdAd) vectors, hybrid adenoviral vectors, herpes simplex virus-based vectors, poxvirus-based vectors, and Epstein-Barr virus-based vectors. Other embodiments of viral vectors and viral particles produce therefrom are described elsewhere herein. In some embodiments, the viral vectors are configured to produce replication incompetent viral particles for improved safety of these systems.

In certain embodiments, the virus structural component, which can be encoded by one or more polynucleotides in a viral vector or vector system, comprises one or more capsid proteins including an entire capsid. In certain embodiments, such as wherein a viral capsid comprises multiple copies of different proteins, the delivery system can provide one or more of the same protein or a mixture of such proteins. For example, AAV comprises 3 capsid proteins, VP1, VP2, and VP3, thus delivery systems of the invention can comprise one or more of VP1, and/or one or more of VP2, and/or one or more of VP3. Accordingly, the present invention is applicable to a virus within the family Adenoviridae, such as Atadenovirus, e.g., Ovine atadenovirus D, Aviadenovirus, e.g., Fowl aviadenovirus A, Ichtadenovirus, e.g., Sturgeon ichtadenovirus A, Mastadenovirus (which includes adenoviruses such as all human adenoviruses), e.g., Human mastadenovirus C, and Siadenovirus, e.g., Frog siadenovirus A. Thus, a virus of within the family Adenoviridae is contemplated as within the invention with discussion herein as to adenovirus applicable to other family members. Target-specific AAV capsid variants can be used or selected. Non-limiting examples include capsid variants selected to bind to chronic myelogenous leukemia cells, human CD34 PBPC cells, breast cancer cells, cells of lung, heart, dermal fibroblasts, melanoma cells, stem cell, glioblastoma cells, coronary artery endothelial cells and keratinocytes. See, e.g., Buning et al, 2015, Current Opinion in Pharmacology 24, 94-104. From teachings herein and knowledge in the art as to modifications of adenovirus (see, e.g., U.S. Pat. Nos. 9,410,129, 7,344,872, 7,256,036, 6,911,199, 6,740,525; Matthews, “Capsid-Incorporation of Antigens into Adenovirus Capsid Proteins for a Vaccine Approach,” Mol Pharm, 8 (1): 3-11 (2011)), as well as regarding modifications of AAV, the skilled person can readily obtain a modified adenovirus that has a large payload protein or a CRISPR-protein, despite that heretofore it was not expected that such a large protein could be provided on an adenovirus. And as to the viruses related to adenovirus mentioned herein, as well as to the viruses related to AAV mentioned elsewhere herein, the teachings herein as to modifying adenovirus and AAV, respectively, can be applied to those viruses without undue experimentation from this disclosure and the knowledge in the art.

In some embodiments, the viral vector is configured such that when the cargo is packaged the cargo(s) (e.g., one or more components of the genetic modifying system, including but not limited to a Cas effector), the SCN1B mimetic polypeptide encoding polynucleotide, is external to the capsid or virus particle. In the sense that it is not inside the capsid (enveloped or encompassed with the capsid) but is externally exposed so that it can contact the target genomic DNA. In some embodiments, the viral vector is configured such that all the cargo(s) (e.g., the SCN1B mimetic polypeptide encoding polynucleotide, or other polynucleotides and/or polypeptides) are contained within the capsid after packaging.

### Split Viral Vector Systems

When the viral vector or vector system (be it a retroviral (e.g., AAV) or lentiviral vector) is designed so as to position the cargo(s) (e.g., one or more the SCN1B mimetic polypeptide encoding polynucleotide(s) and/or genetic modifying system) at the internal surface of the capsid once formed, the cargo(s) will fill most or all of internal volume of the capsid. In other embodiments, the genetic modifying effector (e.g., Cas) (or other exogenous gene or protein e.g., the SCN1B mimetic polypeptide and/or encoding polynucleotide) may be modified or divided so as to occupy a less of the capsid internal volume. Accordingly, in certain embodiments, the genetic modifying system or component thereof or other exogenous gene or protein can be divided in two portions, which can be packaged in separate viral or viral like particles. In certain embodiments, by splitting the genetic modifying system or component thereof in two (or more) portions, space is made available to link one or more heterologous domains to one or both genetic modifying system component or other protein portions (e.g., the SCN1B mimetic polypeptide). Such systems can be referred to as “split vector systems”. This split protein approach is also described elsewhere herein. When the concept is applied to a vector system, it thus describes putting pieces of the split proteins on different vectors thus reducing the payload of any one vector. This approach can facilitate delivery of systems where the total system size is close to or exceeds the packaging capacity of the vector. This is independent of any regulation of the genetic modifying system (e.g., a CRISPR-Cas) system that can be achieved with a split system or split protein design.

In some embodiments, the proteins or peptides described herein can be provided as a split protein or peptide. In some embodiments, polynucleotides encoding the split protein or peptide can be incorporated into a vector or vector system described herein. In certain embodiments, each part of a split protein are attached to a member of a specific binding pair, and when bound with each other, the members of the specific binding pair maintain the parts of the spit protein in proximity. In certain embodiments, each part of a split protein is associated with an inducible binding pair. An inducible binding pair is one which is capable of being switched “on” or “off” by a protein or small molecule that binds to both members of the inducible binding pair. In general, according to the invention, some proteins may preferably split between domains, leaving domains intact.

In some embodiments, monomers of a dimer SCN1B mimetic polypeptide may be split into separate vectors in a split system. After expression from the vectors, dimerization occurs.

### Retroviral and Lentiviral Vectors

Retroviral vectors can be composed of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Suitable retroviral vectors for the delivery of a cargo (e.g., a genetic modifying systems or other exogenous polynucleotide) can include, but are not limited to, those vectors based upon murine leukemia virus (MuLV), gibbon ape leukemia virus (GaLV), Simian immunodeficiency virus (SIV), human immunodeficiency virus (HIV), equine infections anemia (EIA), and combinations thereof (see, e.g., Buchscher et al., J. Virol. 66:2731-2739 (1992); Johann et al., J. Virol. 66:1635-1640 (1992); Sommnerfelt et al., Virol. 176:58-59 (1990); Wilson et al., J. Virol. 63:2374-2378 (1989); Miller et al., J. Virol. 65:2220-2224 (1991); WO1994026877). Other exemplary retroviral vectors are described elsewhere herein.

The tropism of a retrovirus can be altered by incorporating foreign envelope proteins, expanding the potential target population of target cells. Lentiviral vectors are retroviral vectors that are able to transduce or infect non-dividing cells and are described in greater detail elsewhere herein. A retrovirus can also be engineered to allow for conditional expression of the inserted transgene, such that only certain cell types are infected by the lentivirus.

Lentiviruses are complex retroviruses that have the ability to infect and express their genes in both mitotic and post-mitotic cells. Advantages of using a lentiviral approach can include the ability to transduce or infect non-dividing cells and their ability to typically produce high viral titers, which can increase efficiency or efficacy of production and delivery. Exemplary lentiviral vectors include, but are not limited to, human immunodeficiency virus (HIV)-based lentiviral vectors, feline immunodeficiency virus (FIV)-based lentiviral vectors, simian immunodeficiency virus (SIV)-based lentiviral vectors, Moloney Murine Leukaemia Virus (Mo-MLV), Visna.maedi virus (VMV)-based lentiviral vector, carpine arthritis-encephalitis virus (CAEV)-based lentiviral vector, bovine immune deficiency virus (BIV)-based lentiviral vector, and Equine infectious anemia (EIAV)-based lentiviral vector. In some embodiments, an HIV-based lentiviral vector system can be used. In some embodiments, a FIV-based lentiviral vector system can be used.

In some embodiments, the lentiviral vector is an EIAV-based lentiviral vector or vector system. See e.g., Balagaan, J Gene Med 2006; 8:275-285; Binley et al., HUMAN GENE THERAPY 23:980-991 (September 2012)), which can be modified for use with the present disclosure.

In some embodiments, the lentiviral vector or vector system thereof can be a first-generation lentiviral vector or vector system thereof. First-generation lentiviral vectors can contain a large portion of the lentivirus genome, including the gag and pol genes, other additional viral proteins (e.g., VSV-G) and other accessory genes (e.g., vif, vprm vpu, nef, and combinations thereof), regulatory genes (e.g., tat and/or rev) as well as the gene of interest between the LTRs. First generation lentiviral vectors can result in the production of virus particles that can be capable of replication in vivo, which may not be appropriate for some instances or applications.

In some embodiments, the lentiviral vector or vector system thereof can be a second-generation lentiviral vector or vector system thereof. Second-generation lentiviral vectors do not contain one or more accessory virulence factors and do not contain all components necessary for virus particle production on the same lentiviral vector. This can result in the production of a replication-incompetent virus particle and thus increase the safety of these systems over first-generation lentiviral vectors. In some embodiments, the second-generation vector lacks one or more accessory virulence factors (e.g., vif, vprm, vpu, nef, and combinations thereof). Unlike the first-generation lentiviral vectors, no single second generation lentiviral vector includes all features necessary to express and package a polynucleotide into a virus particle. In some embodiments, the envelope and packaging components are split between two different vectors with the gag, pol, rev, and tat genes being contained on one vector and the envelope protein (e.g., VSV-G) are contained on a second vector. The gene of interest, its promoter, and LTRs can be included on a third vector that can be used in conjunction with the other two vectors (packaging and envelope vectors) to generate a replication-incompetent virus particle.

In some embodiments, the lentiviral vector or vector system thereof can be a third-generation lentiviral vector or vector system thereof. Third-generation lentiviral vectors and vector systems thereof have increased safety over first- and second-generation lentiviral vectors and systems thereof because, for example, the various components of the viral genome are split between two or more different vectors but used together in vitro to make virus particles, they can lack the tat gene (when a constitutively active promoter is included upstream of the LTRs), and they can include one or more deletions in the 3′LTR to create self-inactivating (SIN) vectors having disrupted promoter/enhancer activity of the LTR. In some embodiments, a third-generation lentiviral vector system can include (i) a vector plasmid that contains the polynucleotide of interest and upstream promoter that are flanked by the 5′ and 3′ LTRs, which can optionally include one or more deletions present in one or both of the LTRs to render the vector self-inactivating; (ii) a “packaging vector(s)” that can contain one or more genes involved in packaging a polynucleotide into a virus particle that is produced by the system (e.g. gag, pol, and rev) and upstream regulatory sequences (e.g. promoter(s)) to drive expression of the features present on the packaging vector, and (iii) an “envelope vector” that contains one or more envelope protein genes and upstream promoters. In certain embodiments, the third-generation lentiviral vector system can include at least two packaging vectors, with the gag-pol being present on a different vector than the rev gene.

In some embodiments, self-inactivating lentiviral vectors with an siRNA targeting a common exon shared by HIV tat/rev, a nucleolar-localizing TAR decoy, and an anti-CCR5-specific hammerhead ribozyme (see, e.g., DiGiusto et al. (2010) Sci Transl Med 2: 36ra43) can be used/and or adapted to deliver a genetic modifying system or exogenous polynucleotide of the present disclosure.

In some embodiments, the pseudotype and infectivity or tropism of a lentivirus particle can be tuned by altering the type of envelope protein(s) included in the lentiviral vector or system thereof. As used herein, an “envelope protein” or “outer protein” means a protein exposed at the surface of a viral particle that is not a capsid protein. For example, envelope or outer proteins typically comprise proteins embedded in the envelope of the virus. In some embodiments, a lentiviral vector or vector system thereof can include a VSV-G envelope protein. VSV-G mediates viral attachment to an LDL receptor (LDLR) or an LDLR family member present on a host cell, which triggers endocytosis of the viral particle by the host cell. Because LDLR is expressed by a wide variety of cells, viral particles expressing the VSV-G envelope protein can infect or transduce a wide variety of cell types. Other suitable envelope proteins can be incorporated based on the host cell that a user desires to be infected by a virus particle produced from a lentiviral vector or system thereof described herein and can include, but are not limited to, feline endogenous virus envelope protein (RD114) (see e.g., Hanawa et al. Molec. Ther. 2002 5 (3) 242-251), modified Sindbis virus envelope proteins (see e.g., Morizono et al. 2010. J. Virol. 84 (14) 6923-6934; Morizono et al. 2001. J. Virol. 75:8016-8020; Morizono et al. 2009. J. Gene Med. 11:549-558; Morizono et al. 2006 Virology 355:71-81; Morizono et al J. Gene Med. 11:655-663, Morizono et al. 2005 Nat. Med. 11:346-352), baboon retroviral envelope protein (see e.g., Girard-Gagnepain et al. 2014. Blood. 124:1221-1231); Tupaia paramyxovirus glycoproteins (see e.g., Enkirch T. et al., 2013. Gene Ther. 20:16-23); measles virus glycoproteins (see e.g., Funke et al. 2008. Molec. Ther. 16 (8): 1427-1436), rabies virus envelope proteins, MLV envelope proteins, Ebola envelope proteins, baculovirus envelope proteins, filovirus envelope proteins, hepatitis E1 and E2 envelope proteins, gp41 and gp120 of HIV, hemagglutinin, neuraminidase, M2 proteins of influenza virus, and combinations thereof.

In some embodiments, the tropism of the resulting lentiviral particle can be tuned by incorporating cell targeting peptides into a lentiviral vector such that the cell targeting peptides are expressed on the surface of the resulting lentiviral particle. In some embodiments, a lentiviral vector can contain an envelope protein that is fused to a cell targeting protein (see e.g., Buchholz et al. 2015. Trends Biotechnol. 33:777-790; Bender et al. 2016. PLOS Pathog. 12 (e1005461); and Friedrich et al. 2013. Mol. Ther. 2013. 21:849-859).

In some embodiments, a split-intein-mediated approach to target lentiviral particles to a specific cell type can be used (see e.g., Chamoun-Emaneulli et al. 2015. Biotechnol. Bioeng. 112:2611-2617, Ramirez et al. 2013. Protein. Eng. Des. Sel. 26:215-233. In these embodiments, a lentiviral vector can contain one half of a splicing-deficient variant of the naturally split intein from Nostoc punctiforme fused to a cell targeting peptide and the same or different lentiviral vector can contain the other half of the split intein fused to an envelope protein, such as a binding-deficient, fusion-competent virus envelope protein. This can result in production of a virus particle from the lentiviral vector or vector system that includes a split intein that can function as a molecular Velcro linker to link the cell-binding protein to the pseudotyped lentivirus particle. This approach can be advantageous for use where surface-incompatibilities can restrict the use of, e.g., cell targeting peptides.

In some embodiments, a covalent-bond-forming protein-peptide pair can be incorporated into one or more of the lentiviral vectors described herein to conjugate a cell targeting peptide to the virus particle (see e.g., Kasaraneni et al. 2018. Sci. Reports (8) No. 10990). In some embodiments, a lentiviral vector can include an N-terminal PDZ domain of InaD protein (PDZ1) and its pentapeptide ligand (TEFCA) from NorpA, which can conjugate the cell targeting peptide to the virus particle via a covalent bond (e.g., a disulfide bond). In some embodiments, the PDZ1 protein can be fused to an envelope protein, which can optionally be binding deficient and/or fusion competent virus envelope protein and included in a lentiviral vector. In some embodiments, the TEFCA can be fused to a cell targeting peptide and the TEFCA-CPT fusion construct can be incorporated into the same or a different lentiviral vector as the PDZ1-envenlope protein construct. During virus production, specific interaction between the PDZ1 and TEFCA facilitates producing virus particles covalently functionalized with the cell targeting peptide and thus capable of targeting a specific cell-type based upon a specific interaction between the cell targeting peptide and cells expressing its binding partner. This approach can be advantageous for use where surface-incompatibilities can restrict the use of, e.g., cell targeting peptides.

Various exemplary lentiviral vectors, such as those used in the treatment of Parkinson's disease, ocular diseases, delivery to the brain, are described in e.g., US Patent Publication No. 20120295960, 20060281180, 20090007284, US20110117189; US20090017543; US20070054961, US20100317109, US20110293571; US20110293571, US20040013648, US20070025970, US20090111106, and US Patent Nos. U.S. Pat. Nos. 7,259,015, 7,303,910 and 7,351,585. Any of these systems can be used or adapted to deliver a genetic modifying system polynucleotide or other exogenous polynucleotide of the present disclosure.

In some embodiments, a lentiviral vector system can include one or more transfer plasmids. Transfer plasmids can be generated from various other vector backbones and can include one or more features that can work with other retroviral and/or lentiviral vectors in the system that can, for example, improve safety of the vector and/or vector system, increase virial titers, and/or increase or otherwise enhance expression of the desired insert to be expressed and/or packaged into the viral particle. Suitable features that can be included in a transfer plasmid can include, but are not limited to, 5′LTR, 3′LTR, SIN/LTR, origin of replication (Ori), selectable marker genes (e.g., antibiotic resistance genes), Psi (Y), RRE (rev response element), cPPT (central polypurine tract), promoters, WPRE (woodchuck hepatitis post-transcriptional regulatory element), SV40 polyadenylation signal, pUC origin, SV40 origin, F1 origin, and combinations thereof.

In another embodiment, the viral vector is a Cocal vesiculovirus envelope pseudotyped retroviral or lentiviral vector particles are contemplated (see, e.g., US Patent Publication No. 20120164118). Cocal virus is in the Vesiculovirus genus and is a causative agent of vesicular stomatitis in mammals, and as such vectors based on this virus can be used to deliver cells to a wide variety of animals, including insects, cattle, and horses (see e.g., Jonkers et al., Am. J. Vet. Res. 25:236-242 (1964) and Travassos da Rosa et al., Am. J. Tropical Med. & Hygiene 33:999-1006 (1984)). In some embodiments, Cocal vesiculovirus envelope pseudotyped retroviral vector particles may include for example, lentiviral, alpharetroviral, betaretroviral, gammaretroviral, deltaretroviral, and epsilonretroviral vector particles that may comprise retroviral Gag, Pol, and/or one or more accessory protein(s) and a Cocal vesiculovirus envelope protein. In certain embodiments of these embodiments, the Gag, Pol, and accessory proteins are lentiviral and/or gammaretroviral. In some embodiments, a retroviral vector can contain encoding polypeptides for one or more Cocal vesiculovirus envelope proteins such that the resulting viral or pseudoviral particles are Cocal vesiculovirus envelope pseudotyped.

### Adenoviral Vectors, Helper-Dependent Adenoviral Vectors, and Hybrid Adenoviral Vectors

In some embodiments, the vector can be an adenoviral vector. In some embodiments, the adenoviral vector can include elements such that the virus particle produced using the vector or system thereof can be any suitable serotype, such as serotype 2, 5, 8, 9, and others. In some embodiments, the polynucleotide to be delivered via the adenoviral particle can be up to about 8 kb. Thus, in some embodiments, an adenoviral vector can include a DNA polynucleotide to be delivered that can range in size from about 0.001 kb to about 8 kb. Adenoviral vectors have been used successfully in several contexts (see e.g., Teramato et al. 2000. Lancet. 355:1911-1912; Lai et al. 2002. DNA Cell. Biol. 21:895-913; Flotte et al., 1996. Hum. Gene. Ther. 7:1145-1159; and Kay et al. 2000. Nat. Genet. 24:257-261.

In some embodiments the vector can be a helper-dependent adenoviral vector or system thereof. These are also referred to in the art as “gutless” or “gutted” vectors and are a modified generation of adenoviral vectors (see e.g., Thrasher et al. 2006. Nature. 443: E5-7). In certain embodiments of the helper-dependent adenoviral vector system one vector (the helper) can contain all the viral genes required for replication but contains a conditional gene defect in the packaging domain. The second vector of the system can contain only the ends of the viral genome, one or more exogenous polynucleotides, and the native packaging recognition signal, which can allow selective packaged release from the cells (see e.g., Cideciyan et al. 2009. N Engl J Med. 361:725-727). Helper-dependent adenoviral vector systems have been successful for gene delivery in several contexts (see e.g., Simonelli et al. 2010. J Am Soc Gene Ther. 18:643-650; Cideciyan et al. 2009. N Engl J Med. 361:725-727; Crane et al. 2012. Gene Ther. 19 (4): 443-452; Alba et al. 2005. Gene Ther. 12:18-S27; Croyle et al. 2005. Gene Ther. 12:579-587; Amalfitano et al. 1998. J. Virol. 72:926-933; and Morral et al. 1999. PNAS. 96:12816-12821). The techniques and vectors described in these publications can be adapted for inclusion and delivery of the polynucleotides described herein. In some embodiments, the polynucleotide to be delivered via the viral particle produced from a helper-dependent adenoviral vector or system thereof can be up to about 37 kb. Thus, in some embodiments, an adenoviral vector can include a DNA polynucleotide to be delivered that can range in size from about 0.001 kb to about 37 kb (see e.g., Rosewell et al. 2011. J. Genet. Syndr. Gene Ther. Suppl. 5:001).

In some embodiments, the vector is a hybrid-adenoviral vector or system thereof. Hybrid adenoviral vectors are composed of the high transduction efficiency of a gene-deleted adenoviral vector and the long-term genome-integrating potential of adeno-associated, retroviruses, lentivirus, and transposon based-gene transfer. In some embodiments, such hybrid vector systems can result in stable transduction and limited integration site. See e.g., Balague et al. 2000. Blood. 95:820-828; Morral et al. 1998. Hum. Gene Ther. 9:2709-2716; Kubo and Mitani. 2003. J. Virol. 77 (5): 2964-2971; Zhang et al. 2013. PloS One. 8 (10) e76771; and Cooney et al. 2015. Mol. Ther. 23 (4): 667-674), whose techniques and vectors described therein can be modified and adapted for use to deliver a polynucleotide or system of the present invention. In some embodiments, a hybrid-adenoviral vector can include one or more features of a retrovirus and/or an adeno-associated virus. In some embodiments the hybrid-adenoviral vector can include one or more features of a spuma retrovirus or foamy virus (FV). See e.g., Ehrhardt et al. 2007. Mol. Ther. 15:146-156 and Liu et al. 2007. Mol. Ther. 15:1834-1841, whose techniques and vectors described therein can be modified and adapted for use in the polynucleotides and polypeptides of the present invention. Advantages of using one or more features from the FVs in the hybrid-adenoviral vector or system thereof can include the ability of the viral particles produced therefrom to infect a broad range of cells, a large packaging capacity as compared to other retroviruses, and the ability to persist in quiescent (non-dividing) cells. See also e.g., Ehrhardt et al. 2007. Mol. Ther. 156:146-156 and Shuji et al. 2011. Mol. Ther. 19:76-82, whose techniques and vectors described therein can be modified and adapted for use with the polynucleotides and polypeptides of the present invention.

### Adeno Associated Viral (AAV) Vectors

In an embodiment, the vector can be an adeno-associated virus (AAV) vector. See, e.g., West et al., Virology 160:38-47 (1987); U.S. Pat. No. 4,797,368; WO 93/24641; Kotin, Human Gene Therapy 5:793-801 (1994); and Muzyczka, J. Clin. Invest. 94:1351 (1994). Although similar to adenoviral vectors in some of their features, AAVs have some deficiency in their replication and/or pathogenicity and thus can be safer that adenoviral vectors. In some embodiments, the AAV can integrate into a specific site on chromosome 19 of a human cell with no observable side effects. In some embodiments, the capacity of the AAV vector, system thereof, and/or AAV particles can be up to about 4.7 kb. The AAV vector or system thereof can include one or more regulatory molecules. In some embodiments, the regulatory molecules can be promoters, enhancers, repressors and the like, which are described in greater detail elsewhere herein. In some embodiments, the AAV vector or system thereof can include one or more polynucleotides that can encode one or more regulatory proteins. In some embodiments, the one or more regulatory proteins can be selected from Rep78, Rep68, Rep52, Rep40, variants thereof, and combinations thereof.

The AAV vector or system thereof can include one or more polynucleotides that can encode one or more capsid proteins. The capsid proteins can be selected from VP1, VP2, VP3, and combinations thereof. The capsid proteins can be capable of assembling into a protein shell of the AAV virus particle. In some embodiments, the AAV capsid can contain 60 capsid proteins. In some embodiments, the ratio of VP1: VP2: VP3 in a capsid can be about 1:1:10.

In some embodiments, the AAV vector or system thereof can include one or more adenovirus helper factors or polynucleotides that can encode one or more adenovirus helper factors. Such adenovirus helper factors can include, but are not limited, E1A, EIB, E2A, E4ORF6, and VA RNAs. In some embodiments, a producing host cell line expresses one or more of the adenovirus helper factors.

The AAV vector or system thereof can be configured to produce AAV particles having a specific serotype.

AAV particles, packaging polynucleotides encoding compositions of the present disclosure may comprise or be derived from any natural or recombinant AAV serotype. According to the present disclosure, the AAV particles may utilize or be based on a serotype selected from any of the following serotypes, and variants thereof including, but not limited to, AAV1, AAV10, AAV106.1/hu.37, AAV11, AAV114.3/hu.40, AAV12, AAV127.2/hu.41, AAV127.5/hu.42, AAV128.1/hu.43, AAV128.3/hu.44, AAV130.4/hu.48, AAV145.1/hu.53, AAV145.5/hu.54, AAV145.6/hu.55, AAV16.12/hu.11, AAV16.3, AAV16.8/hu.10, AAV161.10/hu.60, AAV161.6/hu.61, AAV1-7/rh.48, AAV1-8/rh.49, AAV2, AAV2.5T, AAV2-15/rh.62, AAV223.1, AAV223.2, AAV223.4, AAV223.5, AAV223.6, AAV223.7, AAV2-3/rh.61, AAV24.1, AAV2-4/rh.50, AAV2-5/rh.51, AAV27.3, AAV29.3/bb.1, AAV29.5/bb.2, AAV2G9, AAV-2-pre-miRNA-101, AAV3, AAV3.1/hu.6, AAV3.1/hu.9, AAV3-11/rh.53, AAV3-3, AAV33.12/hu.17, AAV33.4/hu.15, AAV33.8/hu.16, AAV3-9/rh.52, AAV3a, AAV3b, AAV4, AAV4-19/rh.55, AAV42.12, AAV42-10, AAV42-11, AAV42-12, AAV42-13, AAV42-15, AAV42-1b, AAV42-2, AAV42-3a, AAV42-3b, AAV42-4, AAV42-5a, AAV42-5b, AAV42-6b, AAV42-8, AAV42-aa, AAV43-1, AAV43-12, AAV43-20, AAV43-21, AAV43-23, AAV43-25, AAV43-5, AAV4-4, AAV44.1, AAV44.2, AAV44.5, AAV46.2/hu.28, AAV46.6/hu.29, AAV4-8/r11.64, AAV4-8/rh.64, AAV4-9/rh.54, AAV5, AAV52.1/hu.20, AAV52/hu.19, AAV5-22/rh.58, AAV5-3/rh.57, AAV54.1/hu.21, AAV54.2/hu.22, AAV54.4R/hu.27, AAV54.5/hu.23, AAV54.7/hu.24, AAV58.2/hu.25, AAV6, AAV6.1, AAV6.1.2, AAV6.2, AAV7, AAV7.2, AAV7.3/hu.7, AAV8, AAV-8b, AAV-8h, AAV9, AAV9.11, AAV9.13, AAV9.16, AAV9.24, AAV9.45, AAV9.47, AAV9.61, AAV9.68, AAV9.84, AAV9.9, AAVA3.3, AAVA3.4, AAVA3.5, AAVA3.7, AAV-b, AAVC1, AAVC2, AAVC5, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAV-DJ, AAV-DJ8, AAVF3, AAVF5, AAV-h, AAVH-1/hu.1, AAVH2, AAVH-5/hu.3, AAVH6, AAVhE1.1, AAVhER1.14, AAVhEr1.16, AAVhEr1.18, AAVhER1.23, AAVhEr1.35, AAVhEr1.36, AAVhEr1.5, AAVhEr1.7, AAVhEr1.8, AAVhEr2.16, AAVhEr2.29, AAVhEr2.30, AAVhEr2.31, AAVhEr2.36, AAVhEr2.4, AAVhEr3.1, AAVhu.1, AAVhu. 10, AAVhu. 11, AAVhu.11, AAVhu.12, AAVhu.13, AAVhu.14/9, AAVhu. 15, AAVhu. 16, AAVhu. 17, AAVhu. 18, AAVhu. 19, AAVhu.2, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.3, AAVhu.31, AAVhu.32, AAVhu.34, AAVhu.35, AAVhu.37, AAVhu.39,

AAVhu.4, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.5, AAVhu.51, AAVhu.52, AAVhu.53, AAVhu.54, AAVhu.55, AAVhu.56, AAVhu.57, AAVhu.58, AAVhu.6, AAVhu.60, AAVhu.61, AAVhu.63, AAVhu.64, AAVhu.66, AAVhu.67, AAVhu.7, AAVhu.8, AAVhu.9, AAVhu.t 19, AAVLG-10/rh.40, AAVLG-4/rh.38, AAVLG-9/hu.39, AAVLG-9/hu.39, AAV-LK01, AAV-LK02, AAVLK03, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK17, AAV-LK18, AAV-LK19, AAVN721-8/rh.43, AAV-PAEC, AAV-PAEC11, AAV-PAEC12, AAV-PAEC2, AAV-PAEC4, AAV-PAEC6, AAV-PAEC7, AAV-PAEC8, AAVpi.1, AAVpi.2, AAVpi.3, AAVrh.10, AAVrh.12, AAVrh. 13, AAVrh.13R, AAVrh. 14, AAVrh.17, AAVrh.18, AAVrh.19, AAVrh.2, AAVrh.20, AAVrh.21, AAVrh.22, AAVrh.23, AAVrh.24, AA Vrh.25, AAVrh.2R, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.43, AAVrh.44, AAVrh.45, AAVrh.46, AAVrh.47, AAVrh.48, AAVrh.48, AAVrh.48.1, AAVrh.48.1.2, AAVrh.48.2, AAVrh.49, AAVrh.50, AAVrh.51, AAVrh.52, AAVrh.53, AAVrh.54, AAVrh.55, AAVrh.56, AAVrh.57, AAVrh.58, AAVrh.59, AAVrh.60, AAVrh.61, AAVrh.62, AAVrh.64, AAVrh.64R1, AAVrh.64R2, AAVrh.65, AAVrh.67, AAVrh.68, AAVrh.69, AAVrh.70, AAVrh.72, AAVrh.73, AAVrh.74, AAVrh.8, AAVrh.8R, AAVrh8R, AAVrh8R A586R mutant, AAVrh8R R533A mutant, BAAV, BNP61 AAV, BNP62 AAV, BNP63 AAV, bovine AAV, caprine AAV, Japanese AAV 10, true type AAV (ttAAV), UPENN AAV 10, AAV-LK16, AAAV, AAV Shuffle 100-1, AAV Shuffle 100-2, AAV Shuffle 100-3, AAV Shuffle 100-7, AAV Shuffle 10-2, AAV Shuffle 10-6, AAV Shuffle 10-8, AAV SM 100-10, AAV SM 100-3, AAV SM 10-1, AAV SM 10-2, and/or AAV SM 10-8.

In some embodiments s, the AAV serotype may be, or have, a mutation in the AAV9 sequence as described by N Pulicherla et al. (Molecular Therapy 19 (6): 1070-1078 (2011)), such as but not limited to, AAV9.9, AAV9.11, AAV9.13, AAV9.16, AAV9.24, AAV9.45, AAV9.47, AAV9.61, AAV9.68, AAV9.84.

In some embodiments, the AAV serotype may be, or have, a sequence as described in U.S. Pat. No. 6,156,303, such as, but not limited to, AAV3B (SEQ ID NO: 1 and 10 of U.S. Pat. No. 6,156,303), AAV6 (SEQ ID NO: 2, 7 and 11 of U.S. Pat. No. 6,156,303), AAV2 (SEQ ID NO: 3 and 8 of U.S. Pat. No. 6,156,303), AAV3A (SEQ ID NO: 4 and 9, of U.S. Pat. No. 6,156,303), or derivatives thereof.

In some embodiments, the serotype may be AAVDJ or a variant thereof, such as AAVDJ8 (or AAV-DJ8), as described by Grimm et al. (Journal of Virology 82 (12): 5887-5911 (2008). The amino acid sequence of AAVDJ8 may comprise two or more mutations in order to remove the heparin binding domain (HBD). As a non-limiting example, the AAV-DJ sequence described as SEQ ID NO: 1 in U.S. Pat. No. 7,588,772 may comprise two mutations: (1) R587Q where arginine (R; Arg) at amino acid 587 is changed to glutamine (Q; Gln) and (2) R590T where arginine (R; Arg) at amino acid 590 is changed to threonine (T; Thr). As another non-limiting example, may comprise three mutations: (1) K406R where lysine (K; Lys) at amino acid 406 is changed to arginine (R; Arg), (2) R587Q where arginine (R; Arg) at amino acid 587 is changed to glutamine (Q; Gln) and (3) R590T where arginine (R; Arg) at amino acid 590 is changed to threonine (T; Thr).

In some embodiments, the AAV serotype may be, or have, a sequence as described in International Publication No. WO2015121501, such as, but not limited to, true type AAV (ttAAV) (SEQ ID NO: 2 of WO2015121501), “UPenn AAV10” (SEQ ID NO: 8 of WO2015/121501), “Japanese AAV10” (SEQ ID NO: 9 of WO2015/121501), or variants thereof.

According to the present disclosure, AAV capsid serotype selection or use may be from a variety of species. In one example, the AAV may be an avian AAV (AAAV). The AAAV serotype may be, or have, a sequence as described in U.S. Pat. No. 9,238,800, such as, but not limited to, AAAV (SEQ ID NO: 1, 2, 4, 6, 8, 10, 12, and 14 of U.S. Pat. No. 9,238,800), or variants thereof.

In one example, the AAV may be a bovine AAV (BAAV). The BAAV serotype may be, or have, a sequence as described in U.S. Pat. No. 9,193,769, such as, but not limited to, BAAV (SEQ ID NO: 1 and 6 of U.S. Pat. No. 9,193,769), or variants thereof. The BAAV serotype may be or have a sequence as described in U.S. Pat. No. 7,427,396, such as, but not limited to, BAAV (SEQ ID NO: 5 and 6 of U.S. Pat. No. 7,427,396), or variants thereof.

In one example, the AAV may be a caprine AAV. The caprine AAV serotype may be, or have, a sequence as described in U.S. Pat. No. 7,427,396, such as, but not limited to, caprine AAV (SEQ ID NO: 3 of U.S. Pat. No. 7,427,396), or variants thereof.

In other examples the AAV may be engineered as a hybrid AAV from two or more parental serotypes. In one example, the AAV may be AAV2G9 which comprises sequences from AAV2 and AAV9. The AAV2G9 AAV serotype may be, or have, a sequence as described in United States Patent Publication No. US2016/0017005.

In one example, the AAV may be a serotype generated by the AAV9 capsid library with mutations in amino acids 390-627 (VP1 numbering) as described by Pulicherla et al. (Molecular Therapy 19 (6): 1070-1078 (2011). The serotype and corresponding nucleotide and amino acid substitutions may be, but is not limited to, AAV9.1 (G1594C; D532H), AAV6.2 (T1418A and T1436X; V473D and I479K), AAV9.3 (T1238A; F413Y), AAV9.4 (T1250C and A1617T; F417S), AAV9.5 (A1235G, A1314T, A1642G, C1760T; Q412R, T548A, A587V), AAV9.6 (T1231A; F411I), AAV9.9 (G1203A, G1785T; W595C), AAV9.10 (A1500G, T1676C; M559T), AAV9.11 (A1425T, A1702C, A1769T; T568P, Q590L), AAV9.13 (A1369C, A1720T; N457H, T574S), AAV9.14 (T1340A, T1362C, T1560C, G1713A; L447H), AAV9.16 (A1775T; Q592L), AAV9.24 (T1507C, T1521G; W503R), AAV9.26 (A1337G, A1769C; Y446C, Q590P), AAV9.33 (A1667C; D556A), AAV9.34 (A1534G, C1794T; N512D), AAV9.35 (A1289T, T1450A, C1494T, A1515T, C1794A, G1816A; Q430L, Y484N, N98K, V6061), AAV9.40 (A1694T, E565V), AAV9.41 (A1348T, T1362C; T450S), AAV9.44 (A1684C, A1701T, A1737G; N562H, K567N), AAV9.45 (A1492T, C1804T; N498Y, L602F), AAV9.46 (G1441C, T1525C, T1549G; G481R, W509R, L517V), 9.47 (G1241A, G1358A, A1669G, C1745T; S414N, G453D, K557E, T582I), AAV9.48 (C1445T, A1736T; P482L, Q579L), AAV9.50 (A1638T, C1683T, T1805A; Q546H, L602H), AAV9.53 (G1301A, A1405C, C1664T, G1811T; R134Q, S469R, A555V, G604V), AAV9.54 (C1531A, T1609A; L511I, L537M), AAV9.55 (T1605A; F535L), AAV9.58 (C1475T, C1579A; T492I, H527N), AAV.59 (T1336C; Y446H), AAV9.61 (A1493T; N498I), AAV9.64 (C1531A, A1617T; L511I), AAV9.65 (C1335T, T1530C, C1568A; A523D), AAV9.68 (C1510A; P504T), AAV9.80 (G1441A; G481R), AAV9.83 (C1402A, A1500T; P468T, E500D), AAV9.87 (T1464C, T1468C; S490P), AAV9.90 (A1196T; Y399F), AAV9.91 (T1316G, A1583T, C1782G, T1806C; L439R, K528I), AAV9.93 (A1273G, A1421G, A1638C, C1712T, G1732A, A1744T, A1832T; S425G, Q474R, Q546H, P571L, G578R, T582S, D611V), AAV9.94 (A1675T; M559L) and AAV9.95 (T1605A; F535L).

In one example, the AAV may be a serotype including at least one AAV capsid CD8+ T-cell epitope. As a non-limiting example, the serotype may be AAV1, AAV2 or AAV8.

In one example, the AAV may be a variant, such as PHP.A or PHP.B as described in Deverman. 2016. Nature Biotechnology. 34 (2): 204-209.

AAV vector serotypes can be matched to target cell types. For example, the following exemplary cell types can be transduced by the indicated AAV serotypes among others.

In some embodiments, the serotype can be AAV-1, AAV-2, AAV-3, AAV-4, AAV-5, AAV-6, AAV-8, AAV-9 or any combinations thereof. In some embodiments, the AAV can be AAV1, AAV-2, AAV-5 or any combination thereof. One can select the AAV of the AAV with regard to the cells to be targeted; e.g., one can select AAV serotypes 1, 2, 5 or a hybrid capsid AAV-1, AAV-2, AAV-5 or any combination thereof for targeting brain and/or neuronal cells; and one can select AAV-4 for targeting cardiac tissue; and one can select AAV8 for delivery to the liver. Thus, in some embodiments, an AAV vector or system thereof capable of producing AAV particles capable of targeting the brain and/or neuronal cells can be configured to generate AAV particles having serotypes 1, 2, 5 or a hybrid capsid AAV-1, AAV-2, AAV-5 or any combination thereof. In some embodiments, an AAV vector or system thereof capable of producing AAV particles capable of targeting cardiac tissue can be configured to generate an AAV particle having an AAV-4 serotype. In some embodiments, an AAV vector or system thereof capable of producing AAV particles capable of targeting the liver can be configured to generate an AAV having an AAV-8 serotype. In some embodiments, the AAV vector is a hybrid AAV vector or system thereof. Hybrid AAVs are AAVs that include genomes with elements from one serotype that are packaged into a capsid derived from at least one different serotype. For example, if it is the rAAV2/5 that is to be produced, and if the production method is based on the helper-free, transient transfection method discussed above, the 1st plasmid and the 3rd plasmid (the adeno helper plasmid) will be the same as discussed for rAAV2 production. However, the second plasmid, the pRepCap will be different. In this plasmid, called pRep2/Cap5, the Rep gene is still derived from AAV2, while the Cap gene is derived from AAV5. The production scheme is the same as the above-mentioned approach for AAV2 production. The resulting rAAV is called rAAV2/5, in which the genome is based on recombinant AAV2, while the capsid is based on AAV5. It is assumed the cell or tissue-tropism displayed by this AAV2/5 hybrid virus should be the same as that of AAV5.

A tabulation of certain AAV serotypes as to these cells can be found in Grimm, D. et al, J. Virol. 82:5887-5911 (2008).

In some embodiments, the AAV vector or system thereof is configured as a “gutless” vector, similar to that described in connection with a retroviral vector. In some embodiments, the “gutless” AAV vector or system thereof can have the cis-acting viral DNA elements involved in genome amplification and packaging in linkage with the heterologous sequences of interest (e.g., the genetic modifying system polynucleotide(s)).

In some embodiments, the AAV vectors are produced in in insect cells, e.g., Spodoptera frugiperda Sf9 insect cells, grown in serum-free suspension culture. Serum-free insect cells can be purchased from commercial vendors, e.g., Sigma Aldrich (EX-CELL 405).

In some embodiments, an AAV vector or vector system can contain or consists essentially of one or more polynucleotides encoding one or more components of a genetic modifying system or other exogenous polynucleotide to be delivered to a cell. Specific cassette configuration for delivery of a genetic modifying system and/or other exogenous polynucleotide(s) will be appreciated by one of ordinary skill in the art in view of the description herein.

In some embodiments, one or more components of a genetic modifying system or other polypeptides and/or polynucleotides are associated with Adeno Associated Virus (AAV), e.g., an AAV comprising a polypeptide of the genetic modification system or exogenous polypeptide as a fusion, with or without a linker, to or with an AAV capsid protein such as VP1, VP2, and/or VP3. More in particular, modifying the knowledge in the art, e.g., Rybniker et al., “Incorporation of Antigens into Viral Capsids Augments Immunogenicity of Adeno-Associated Virus Vector-Based Vaccines,” J Virol. December 2012; 86 (24): 13800-13804, Lux K, et al. 2005. Green fluorescent protein-tagged adeno-associated virus particles allow the study of cytosolic and nuclear trafficking. J. Virol. 79:11776-11787, Munch R C, et al. 2012. “Displaying high-affinity ligands on adeno-associated viral vectors enables tumor cell-specific and safe gene transfer.” Mol. Ther. [Epub ahead of print.] doi: 10.1038/mt.2012.186 and Warrington K H, Jr, et al. 2004. Adeno-associated virus type 2 VP2 capsid protein is nonessential and can tolerate large peptide insertions at its N terminus. J. Virol. 78:6595-6609, each incorporated herein by reference, one can obtain a modified AAV capsid as described herein. It will be understood by those skilled in the art that the modifications described herein if inserted into the AAV cap gene may result in modifications in the VP1, VP2 and/or VP3 capsid subunits. Alternatively, the capsid subunits can be expressed independently to achieve modification in only one or two of the capsid subunits (VP1, VP2, VP3, VP1+VP2, VP1+VP3, or VP2+VP3). One can modify the cap gene to have expressed at a desired location a non-capsid protein advantageously a large payload protein, such as a polypeptide of the present invention. Likewise, these can be fusions, with the protein, e.g., large payload protein such as a large protein described herein fused in a manner analogous to prior art fusions. See, e.g., US Patent Publication 20090215879; Nance et al., “Perspective on Adeno-Associated Virus Capsid Modification for Duchenne Muscular Dystrophy Gene Therapy,” Hum Gene Ther. 26 (12): 786-800 (2015) and documents cited therein, incorporated herein by reference. The skilled person, from this disclosure and the knowledge in the art can make and use modified AAV or AAV capsid as with other aspects of the present disclosure, and through this description herein one knows now that large payload proteins can be fused to the AAV capsid. Accordingly, the approaches described herein are also applicable to a virus in the genus Dependoparvovirus or in the family Parvoviridae, for instance, AAV, or a virus of Amdoparvovirus, e.g., Carnivore amdoparvovirus 1, a virus of Aveparvovirus, e.g., Galliform aveparvovirus 1, a virus of Bocaparvovirus, e.g., Ungulate bocaparvovirus 1, a virus of Copiparvovirus, e.g., Ungulate copiparvovirus 1, a virus of Dependoparvovirus, e.g., Adeno-associated dependoparvovirus A, a virus of Erythroparvovirus, e.g., Primate erythroparvovirus 1, a virus of Protoparvovirus, e.g., Rodent protoparvovirus 1, a virus of Tetraparvovirus, e.g., Primate tetraparvovirus 1.

In some embodiments, a genetic modifying system polypeptide or other exogenous polypeptide is external to the capsid or virus particle, such as an AAV capsid. Although this approach is discussed in the context of AAVs, such an approach is applicable to other viral systems or viral like particle systems where capsids are formed. In these embodiments, the cargo polypeptide is not inside the capsid (enveloped or encompassed with the capsid), but is externally exposed so that it can contact the target genomic or other target DNA or RNA). In some embodiments, the cargo polypeptide is associated with the AAV VP2 domain by way of a fusion protein. In some embodiments, the association may be considered to be a modification of the VP2 domain. In some embodiments, the AAV VP2 domain may be associated (or tethered) to a cargo polypeptide via a connector protein, for example using a system such as the streptavidin-biotin system. Also provided herein are polynucleotides encoding a cargo polypeptide (e.g., a genetic modifying polypeptide or other exogenous cargo polypeptide) and associated AAV VP2 domain. In some preferred embodiments, the cargo polypeptide is fused or tethered (e.g., via linker) to the VP2 domain so that, a non-naturally occurring modified

AAV having a VP2-cargo polypeptide fusion or otherwise modified capsid protein is formed. In some embodiments, where the cargo is tethered via a linker, the cargo can be distanced from the remainder of the AAV (or other viral or viral like particle). The fusion or tether can be at the N-terminus, C-terminus, or both of the capsid polypeptide. In some embodiments, an NLS and/or a linker (such as a GlySer linker) or other tether is positioned between the C-terminal end of the cargo and the N-terminal end of the capsid domain. In some embodiments, an NLS and/or a linker (such as a GlySer linker) or other tether is positioned between the N-terminal end of the cargo and the C-terminal end of the capsid domain. In some embodiments, the capsid polypeptide that is modified with a cargo polypeptide is truncated or contains a loss of one or more internal amino acids with the N- and C-terminal amino acids (e.g., the first (or last) 2-10 amino acids of the capsid domain intact. In these embodiments, the cargo polypeptide can be inserted between the intact N-and/or C-terminal amino acids via a fusion (e.g., an in-frame fusion) or linker or other tether (such as a streptavidin/biotin system or other adaptor molecule such as MS2). In some embodiments where a linker is used, the linker can be a branched linker, which can allow for more distance between the cargo polypeptide and capsid. A cargo polypeptide can be incorporated into other capsid domains of the AAV (e.g., VP1 and/or VP3) in a similar fashion as described with respect to VP2. Likewise, similar approaches (e.g., fusion or tethered) can be used to modified non-AAV capsids of other viral and viral-like delivery systems described herein.

### Herpes Simplex Viral Vectors

In some embodiments, the vector is a Herpes Simplex Viral (HSV)-based vector or system thereof. HSV systems can include the disabled infections single copy (DISC) viruses, which are composed of a glycoprotein H defective mutant HSV genome. When the defective HSV is propagated in complementing cells, virus particles can be generated that are capable of infecting subsequent cells permanently replicating their own genome but are not capable of producing more infectious particles. See e.g., 2009. Trobridge. Exp. Opin. Biol. Ther. 9:1427-1436, whose techniques and vectors described therein can be modified and adapted for use in the polynucleotides and polypeptides of the present invention. In some embodiments where an HSV vector or system thereof is utilized, the host cell can be a complementing cell. In some embodiments, HSV vector or system thereof can be capable of producing virus particles capable of delivering a polynucleotide cargo of up to 150 kb. Thus, in some embodiments the cargo polynucleotide(s) included in the HSV-based viral vector or system thereof can sum from about 0.001 to about 150 kb. HSV-based vectors and systems thereof have been successfully used in several contexts including various models of neurologic disorders. See e.g., Cockrell et al. 2007. Mol. Biotechnol. 36:184-204; Kafri T. 2004. Mol. Biol. 246:367-390; Balaggan and Ali. 2012. Gene Ther. 19:145-153; Wong et al. 2006. Hum. Gen. Ther. 2002. 17:1-9; Azzouz et al. J. Neruosci. 22L10302-10312; and Betchen and Kaplitt. 2003. Curr. Opin. Neurol. 16:487-493, whose techniques and vectors described therein can be modified and adapted for use with the present disclosure.

### Poxvirus Vectors

In some embodiments, the vector can be a poxvirus vector or system thereof. In some embodiments, the poxvirus vector can result in cytoplasmic expression of one or more cargo polynucleotides of the present disclosure. In some embodiments the capacity of a poxvirus vector or system thereof can be about 25 kb or more. In some embodiments, a poxvirus vector or system thereof can include one or more cargo polynucleotides described herein.

Virus Particle Production from Viral Vectors

### Retroviral Production

In some embodiments, one or more viral vectors and/or system thereof can be delivered to a suitable cell line for production of virus particles containing the polynucleotide or other payload to be delivered to a host cell. Suitable host cells for virus production from viral vectors and systems thereof described herein are known in the art and are commercially available. For example, suitable host cells include HEK 293 cells and its variants (HEK 293T and HEK 293TN cells). In some embodiments, the suitable host cell for virus production from viral vectors and systems thereof described herein can stably express one or more genes involved in packaging (e.g., pol, gag, and/or VSV-G) and/or other supporting genes.

In some embodiments, after delivery of one or more viral vectors to the suitable host cells for or virus production from viral vectors and systems thereof, the cells are incubated for an appropriate length of time to allow for viral gene expression from the vectors, packaging of the polynucleotide to be delivered (e.g., a genetic modifying system polynucleotide or other polynucleotide of the present disclosure), and virus particle assembly, and secretion of mature virus particles into the culture media. Various other methods and techniques are generally known to those of ordinary skill in the art.

Mature virus particles can be collected from the culture media by a suitable method. In some embodiments, this can involve centrifugation to concentrate the virus. The titer of the composition containing the collected virus particles can be obtained using a suitable method. Such methods can include transducing a suitable cell line (e.g., NIH 3T3 cells) and determining transduction efficiency, infectivity in that cell line by a suitable method. Suitable methods include PCR-based methods, flow cytometry, and antibiotic selection-based methods. Various other methods and techniques are generally known to those of ordinary skill in the art. The concentration of virus particle can be adjusted as needed. In some embodiments, the resulting composition containing virus particles can contain 1×10¹-1×10²⁰ or more particles/mL.

Lentiviruses may be prepared from any lentiviral vector or vector system described herein. In one example embodiment, after cloning a polynucleotide to be delivered into a suitable lentiviral vector (which contains a lentiviral transfer plasmid backbone), HEK293FT at low passage (p=5) can be seeded in a T-75 flask to 50% confluence the day before transfection in DMEM with 10% fetal bovine serum and without antibiotics. After 20 hours, the media can be changed to OptiMEM (serum-free) media and transfection of the lentiviral vectors can done 4 hours later. Cells can be transfected with 10 μg of lentiviral transfer plasmid (pCasES10) and the appropriate packaging plasmids (e.g., 5 μg of pMD2.G (VSV-g pseudotype), and 7.5 ug of psPAX2 (gag/pol/rev/tat)). Transfection can be carried out in 4 mL OptiMEM with a cationic lipid delivery agent (50 uL Lipofectamine 2000 and 100 ul Plus reagent). After 6 hours, the media can be changed to antibiotic-free DMEM with 10% fetal bovine serum. These methods can use serum during cell culture, but serum-free methods are preferred.

Following transfection and allowing the producing cells (also referred to as packaging cells) to package and produce virus particles with packaged cargo, the lentiviral particles can be purified. In an exemplary embodiment, virus-containing supernatants can be harvested after 48 hours. Collected virus-containing supernatants can first be cleared of debris and filtered through a 0.45 μm low protein binding (PVDF) filter. They can then be spun in an ultracentrifuge for 2 hours at 24,000 rpm. The resulting virus-containing pellets can be resuspended in 50 ul of DMEM overnight at 4 degrees C. They can be then aliquoted and used immediately or immediately frozen at −80 degrees C. for storage.

See also Merten et al., 2016. “Production of lentiviral vectors.” Mol. Ther. 3:10617 for additional methods and techniques for lentiviral vector and particle production, which can be adapted for use with the present disclosure.

### AAV Particle Production

General principles of rAAV production are reviewed in, for example, Carter, 1992, Current Opinions in Biotechnology, 1533-539; and Muzyczka, 1992, Curr. Topics in Microbial. and Immunol., 158:97-129). Various approaches are described in Ratschin et al., Mol. Cell. Biol. 4:2072 (1984); Hermonat et al., Proc. Natl. Acad. Sci. USA, 81:6466 (1984); Tratschin et al., Mol. Cell. Biol. 5:3251 (1985); McLaughlin et al., J. Virol., 62:1963 (1988); and Lebkowski et al., 1988 Mol. Cell. Biol., 7:349 (1988). Samulski et al. (1989, J. Virol., 63:3822-3828); U.S. Pat. No. 5,173,414; WO 95/13365 and corresponding U.S. Pat. No. 5,658,776; WO 95/13392; WO 96/17947; PCT/US98/18600; WO 97/09441 (PCT/US96/14423); WO 97/08298 (PCT/US96/13872); WO 97/21825 (PCT/US96/20777); WO 97/06243 (PCT/FR96/01064); WO 99/11764; Perrin et al. (1995) Vaccine 13:1244-1250; Paul et al. (1993) Human Gene Therapy 4:609-615; Clark et al. (1996) Gene Therapy 3:1124-1132; U.S. Pat. Nos. 5,786,211; 5,871,982; and 6,258,595.

In general, there are two main strategies for producing AAV particles from AAV vectors and systems thereof, such as those described herein, which depend on how the adenovirus helper factors are provided (helper v, helper free). In some embodiments, a method of producing AAV particles from AAV vectors and systems thereof can include adenovirus infection into cell lines that stably harbor AAV replication and capsid encoding polynucleotides along with AAV vector containing the cargo polynucleotide to be packaged and delivered by the resulting AAV particle (e.g., the genetic modifying system polynucleotide(s)). In some embodiments, a method of producing AAV particles from AAV vectors and systems thereof can be a “helper free” method, which includes co-transfection of an appropriate producing cell line with three vectors (e.g., plasmid vectors): (1) an AAV vector that contains a cargo polynucleotide (e.g., the polynucleotide(s)) encoding polypeptides of the present invention between 2 ITRs; (2) a vector that carries the AAV Rep-Cap encoding polynucleotides; and (helper polynucleotides). One of skill in the art will appreciate various methods and variations thereof that are both helper and -helper free and as well as the different advantages of each system. See also Kimur et al., 2019. Sci. Rep. 6:13601; Shin et al., Meth. Mol Biol. 2012. 798:267-284; Negrini et al., 2020. Curr. Prot. Neurosci. 93: e103; Dobrowsky et al., 2021. Curr. Op. Biomed. Eng. 20:100353 for additional methods and techniques for AAV vector and particle production, which can be adapted for use with the present disclosure.

### Non-Viral Vectors

In some embodiments, the vector is a non-viral vector or vector system. The term of art “non-viral vector” and as used herein in this context refers to molecules and/or compositions that are vectors but that are not based on one or more component of a virus or virus genome (excluding any nucleotide to be delivered and/or expressed by the non-viral vector) that can be capable of incorporating cargo polynucleotide(s) and delivering said cargo polynucleotide(s) to a cell and/or expressing the polynucleotide in the cell. It will be appreciated that this does not exclude vectors containing a polynucleotide designed to target a virus-based polynucleotide that is to be delivered. For example, if a gRNA to be delivered is directed against a virus component and it is inserted or otherwise coupled to an otherwise non-viral vector or carrier, this would not make said vector a “viral vector”. Non-viral vectors can include, without limitation, naked polynucleotides and polynucleotide (non-viral) based vector and vector systems.

### Naked Polynucleotides

In some embodiments one or more polynucleotides of the present disclosure described elsewhere herein can be included in a naked polynucleotide. The term of art “naked polynucleotide” as used herein refers to polynucleotides that are not associated with another molecule (e.g., proteins, lipids, and/or other molecules) that can often help protect it from environmental factors and/or degradation. As used herein, associated with includes, but is not limited to, linked to, adhered to, adsorbed to, enclosed in, enclosed in or within, mixed with, and the like. Naked polynucleotides that include one or more of the cargo polynucleotides described herein can be delivered directly to a host cell and optionally expressed therein. The naked polynucleotides can have any suitable two- and three-dimensional configurations. By way of non-limiting examples, naked polynucleotides can be single-stranded molecules, double stranded molecules, circular molecules (e.g., plasmids and artificial chromosomes), molecules that contain portions that are single stranded and portions that are double stranded (e.g., ribozymes), and the like. In some embodiments, the naked polynucleotide contains only the cargo polynucleotide(s). In some embodiments, the naked polynucleotide can contain other nucleic acids and/or polynucleotides in addition to the cargo polynucleotide(s). The naked polynucleotides can include one or more elements of a transposon system. Transposons and system thereof are described in greater detail elsewhere herein.

### Non-Viral Polynucleotide Vectors

In some embodiments, one or more of the polynucleotides of the present disclosure can be included in a non-viral polynucleotide vector. Suitable non-viral polynucleotide vectors include, but are not limited to, transposon vectors and vector systems, plasmids, bacterial artificial chromosomes, yeast artificial chromosomes, AR (antibiotic resistance)-free plasmids and miniplasmids, circular covalently closed vectors (e.g. minicircles, minivectors, miniknots,), linear covalently closed vectors (“dumbbell shaped”), MIDGE (minimalistic immunologically defined gene expression) vectors, MiLV (micro-linear vector) vectors, Ministrings, mini-intronic plasmids, PSK systems (post-segregationally killing systems), ORT (operator repressor titration) plasmids, and the like. See e.g., Hardee et al. 2017. Genes. 8 (2): 65.

In some embodiments, the non-viral polynucleotide vector can have a conditional origin of replication. In some embodiments, the non-viral polynucleotide vector can be an ORT plasmid. In some embodiments, the non-viral polynucleotide vector can have a minimalistic immunologically defined gene expression. In some embodiments, the non-viral polynucleotide vector can have one or more post-segregationally killing system genes. In some embodiments, the non-viral polynucleotide vector is AR-free. In some embodiments, the non-viral polynucleotide vector is a minivector. In some embodiments, the non-viral polynucleotide vector includes a nuclear localization signal. In some embodiments, the non-viral polynucleotide vector can include one or more CpG motifs. In some embodiments, the non-viral polynucleotide vectors can include one or more scaffold/matrix attachment regions (S/MARs). See e.g., Mirkovitch et al. 1984. Cell. 39:223-232, Wong et al. 2015. Adv. Genet. 89:113-152, whose techniques and vectors can be adapted for use in the present invention. S/MARs are AT-rich sequences that play a role in the spatial organization of chromosomes through DNA loop base attachment to the nuclear matrix. S/MARs are often found close to regulatory elements such as promoters, enhancers, and origins of DNA replication. Inclusion of one or S/MARs can facilitate a once-per-cell-cycle replication to maintain the non-viral polynucleotide vector as an episome in daughter cells. In certain embodiments, the S/MAR sequence is located downstream of an actively transcribed polynucleotide (e.g., one or more cargo polynucleotides) included in the non-viral polynucleotide vector. In some embodiments, the S/MAR can be a S/MAR from the beta-interferon gene cluster. See e.g., Verghese et al. 2014. Nucleic Acid Res. 42: e53; Xu et al. 2016. Sci. China Life Sci. 59:1024-1033; Jin et al. 2016. 8:702-711; Koirala et al. 2014. Adv. Exp. Med. Biol. 801:703-709; and Nehlsen et al. 2006. Gene Ther. Mol. Biol. 10:233-244, whose techniques and vectors can be adapted for use in the present invention.

In some embodiments, the non-viral vector is a transposon vector or system thereof. As used herein, “transposon” (also referred to as transposable element) refers to a polynucleotide sequence that is capable of moving form location in a genome to another. There are several classes of transposons. Transposons include retrotransposons and DNA transposons. Retrotransposons require the transcription of the polynucleotide that is moved (or transposed) in order to transpose the polynucleotide to a new genome or polynucleotide. DNA transposons are those that do not require reverse transcription of the polynucleotide that is moved (or transposed) in order to transpose the polynucleotide to a new genome or polynucleotide. In some embodiments, the non-viral polynucleotide vector can be a retrotransposon vector. In some embodiments, the retrotransposon vector includes long terminal repeats. In some embodiments, the retrotransposon vector does not include long terminal repeats. In some embodiments, the non-viral polynucleotide vector can be a DNA transposon vector. DNA transposon vectors can include a polynucleotide sequence encoding a transposase. In some embodiments, the transposon vector is configured as a non-autonomous transposon vector, meaning that the transposition does not occur spontaneously on its own. In some of these embodiments, the transposon vector lacks one or more polynucleotide sequences encoding proteins required for transposition. In some embodiments, the non-autonomous transposon vectors lack one or more Ac elements.

In some embodiments a non-viral polynucleotide transposon vector system can include a first polynucleotide vector that contains the cargo polynucleotide(s) of the present invention flanked on the 5′ and 3′ ends by transposon terminal inverted repeats (TIRs) and a second polynucleotide vector that includes a polynucleotide capable of encoding a transposase coupled to a promoter to drive expression of the transposase. When both are expressed in the same cell the transposase can be expressed from the second vector and can transpose the material between the TIRs on the first vector (e.g., the cargo polynucleotide(s) of the present invention) and integrate it into one or more positions in the host cell's genome. In some embodiments the transposon vector or system thereof can be configured as a gene trap. In some embodiments, the TIRs can be configured to flank a strong splice acceptor site followed by a reporter and/or other gene (e.g., one or more of the cargo polynucleotide(s) of the present invention) and a strong poly A tail. When transposition occurs while using this vector or system thereof, the transposon can insert into an intron of a gene and the inserted reporter or other gene can provoke a mis-splicing process and as a result it in activates the trapped gene.

Any suitable transposon system can be used. Suitable transposon and systems thereof can include without limitation Sleeping Beauty transposon system (Tcl/mariner superfamily) (see e.g., Ivics et al. 1997. Cell. 91 (4): 501-510), piggyBac (piggyBac superfamily) (see e.g., Li et al. 2013 110 (25): E2279-E2287 and Yusa et al. 2011. PNAS. 108 (4): 1531-1536), Tol2 (superfamily hAT), Frog Prince (Tcl/mariner superfamily) (see e.g., Miskey et al. 2003 Nucleic Acid Res. 31 (23): 6873-6881) and variants thereof.

### Non-Vector Delivery Vehicles

The delivery vehicles may comprise non-vector vehicles. In general, methods and vehicles capable of delivering nucleic acids and/or proteins may be used for delivering the systems compositions herein. Examples of non-vector vehicles include lipid nanoparticles, cell-penetrating peptides (CPPs), DNA nanoclews, metal nanoparticles, streptolysin O, multifunctional envelope-type nanodevices (MENDs), lipid-coated mesoporous silica particles, and other inorganic nanoparticles.

### Lipid Particles

The delivery vehicles can include or be composed of lipid particles, e.g., lipid nanoparticles (LNPs) and liposomes. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Felgner, International Patent Publication Nos. WO 91/17424 and WO 91/16024. The preparation of lipid: nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

### Lipid Nanoparticles (LNPs)

LNPs may encapsulate nucleic acids within cationic lipid particles (e.g., liposomes), and may be delivered to cells with relative ease. In some examples, lipid nanoparticles do not contain any viral components, which helps minimize safety and immunogenicity concerns. Lipid particles may be used for in vitro, ex vivo, and in vivo deliveries. Lipid particles may be used for various scales of cell populations.

In some examples, LNPs may be used for delivering DNA molecules (e.g., those comprising coding sequences of a cargo polypeptide) and/or RNA molecules (e.g., mRNA of encoding a cargo polypeptide and/or other RNA cargos such as gRNAs). In certain cases, LNPs may be use for delivering RNP complexes of e.g., a polypeptide of the present invention.

Components in LNPs may comprise cationic lipids 1,2-dilineoyl-3-dimethylammonium-propane (DLinDAP), 1,2-dilinoleyloxy-3-N,N-dimethylaminopropane (DLinDMA), 1,2-dilinoleyloxyketo-N,N-dimethyl-3-aminopropane (DLinK-DMA), 1,2-dilinoleyl-4-(2-dimethylaminoethyl)-[1,3]-dioxolane (DLinKC2-DMA), (3-O-[2″-(methoxypolyethyleneglycol 2000) succinoyl]-1,2-dimyristoyl-sn-glycol (PEG-S-DMG), R-3-[(ro-methoxy-poly(ethylene glycol) 2000) carbamoyl]-1,2-dimyristyloxlpropyl-3-amine (PEG-C-DOMG, and any combination thereof. Preparation of LNPs and encapsulation may be adapted from Rosin et al, Molecular Therapy, vol. 19, no. 12, pages 1286-220 Dec. 2011).

In some embodiments, an LNP delivery vehicle can be used to deliver a virus particle containing cargo polypeptides or polynucleotides. In some embodiments, the virus particle(s) can be adsorbed to the lipid particle, such as through electrostatic interactions, and/or can be attached to the liposomes via a linker.

In some embodiments, the LNP contains a nucleic acid, wherein the charge ratio of nucleic acid backbone phosphates to cationic lipid nitrogen atoms is about 1:1.5-7 or about 1:4.

In some embodiments, the LNP also includes a shielding compound, which is removable from the lipid composition under in vivo conditions. In some embodiments, the shielding compound is a biologically inert compound. In some embodiments, the shielding compound does not carry any charge on its surface or on the molecule as such. In some embodiments, the shielding compounds are polyethylenglycoles (PEGs), hydroxyethylglucose (HEG) based polymers, polyhydroxyethyl starch (polyHES) and polypropylene. In some embodiments, the PEG, HEG, polyHES, and a polypropylene weight between about 500 to 10,000 Da or between about 2000 to 5000 Da. In some embodiments, the shielding compound is PEG2000 or PEG5000.

In some embodiments, the LNP can include one or more helper lipids. In some embodiments, the helper lipid can be a phosphor lipid or a steroid. In some embodiments, the helper lipid is between about 20 mol % to 80 mol % of the total lipid content of the composition. In some embodiments, the helper lipid component is between about 35 mol % to 65 mol % of the total lipid content of the LNP. In some embodiments, the LNP includes lipids at 50 mol % and the helper lipid at 50 mol % of the total lipid content of the LNP.

Other non-limiting, exemplary LNP delivery vehicles are described in U.S. Patent Publication Nos. US20160174546, US20140301951, US20150105538, US20150250725, Wang et al., J. Control Release, 2017 Jan. 31. pii: S0168-3659 (17) 30038-X. doi: 10.1016/j.jconrel.2017.01.037.; Altnoğlu et al., Biomater Sci., 4 (12): 1773-80, Nov. 15, 2016; Wang et al., PNAS, 113 (11): 2868-73 Mar. 15, 2016; Wang et al., PloS One, 10 (11): e0141860. doi: 10.1371/journal.pone.0141860. eCollection 2015 Nov. 3, 2015; Takeda et al., Neural Regen Res. 10 (5): 689-90, May 2015; Wang et al., Adv. Healthc Mater., 3 (9): 1398-403, September 2014; and Wang et al., Agnew Chem Int Ed Engl., 53 (11): 2893-8, Mar. 10, 2014; James E. Dahlman and Carmen Barnes et al. Nature Nanotechnology (2014) published online 11 May 2014, doi: 10.1038/nnano.2014.84; Coelho et al., N Engl J Med 2013; 369:819-29; Aleku et al., Cancer Res., 68 (23): 9788-98 (Dec. 1, 2008), Strumberg et al., Int. J. Clin. Pharmacol. Ther., 50 (1): 76-8 (January 2012), Schultheis et al., J. Clin. Oncol., 32 (36): 4141-48 (Dec. 20, 2014), and Fehring et al., Mol. Ther., 22 (4): 811-20 (Apr. 22, 2014); Novobrantseva, Molecular Therapy-Nucleic Acids (2012) 1, e4; doi: 10.1038/mtna.2011.3; WO2012135025; US20140348900; U.S. Pat. No. 20,140,328759; US20140308304; WO 2005/105152; WO 2006/069782; WO 2007/121947; US 2015/082080; US20120251618; 7,982,027; 7,799,565; 8,058,069; 8,283,333; 7,901,708; 7,745,651; 7,803,397; 8,101,741; 8,188,263; 7,915,399; 8,236,943 and 7,838,658 and European Pat. Nos 1766035; 1519714; 1781593 and 1664316.

### Liposomes

In some embodiments, a lipid particle may be liposome. Liposomes are spherical vesicle structures composed of a uni- or multilamellar lipid bilayer surrounding internal aqueous compartments and a relatively impermeable outer lipophilic phospholipid bilayer. In some embodiments, liposomes are biocompatible, nontoxic, can deliver both hydrophilic and lipophilic drug molecules, protect their cargo from degradation by plasma enzymes, and transport their load across biological membranes and the blood brain barrier (BBB).

Liposomes can be made from several different types of lipids, e.g., phospholipids. A liposome may comprise natural phospholipids and lipids such as 1,2-distearoryl-sn-glycero-3-phosphatidyl choline (DSPC), sphingomyelin, egg phosphatidylcholines, monosialoganglioside, or any combination thereof.

Several other additives may be added to liposomes in order to modify their structure and properties. For instance, liposomes may further comprise cholesterol, sphingomyelin, and/or 1,2-dioleoyl-sn-glycero-3-phosphoethanolamine (DOPE), e.g., to increase stability and/or to prevent the leakage of the liposomal inner cargo.

In some embodiments, a liposome delivery vehicle can be used to deliver a virus particle containing cargo polypeptide(s) and/or polynucleotide(s). In some embodiments, the virus particle(s) can be adsorbed to the liposome, such as through electrostatic interactions, and/or can be attached to the liposomes via a linker.

In some embodiments, the liposome can be a Trojan Horse liposome (also known in the art as Molecular Trojan Horses), see e.g., cshprotocols.cshlp.org/content/2010/4/pdb.prot5407.long, the teachings of which can be applied and/or adapted to generated and/or deliver the genetic modifying systems and/or other cargo polypeptides or polynucleotides described herein.

Other non-limiting, exemplary liposomes can be those as set forth in Wang et al., ACS Synthetic Biology, 1, 403-07 (2012); Wang et al., PNAS, 113 (11) 2868-2873 (2016); Spuch and Navarro, Journal of Drug Delivery, vol. 2011, Article ID 469679, 12 pages, 2011. doi: 10.1155/2011/469679; WO 2008/042973; U.S. Pat. No. 8,071,082; WO 2014/186366; 20160257951; US20160129120; US20160244761; US20120251618; WO 2013/093648; Lipofectin (a combination of DOTMA and DOPE), Lipofectase, LIPOFECTAMINE.RTM. (e.g., LIPOFECTAMINE.RTM. 2000, LIPOFECTAMINE.RTM. 3000, LIPOFECTAMINE.RTM. RNAIMAX, LIPOFECTAMINE.RTM. LTX), SAINT-RED (Synvolux Therapeutics, Groningen Netherlands), DOPE, Cytofectin (Gilead Sciences, Foster City, Calif.), and Eufectins (JBL, San Luis Obispo, Calif.).

### Stable Nucleic-Acid-Lipid Particles (SNALPs)

In some embodiments, the lipid particles contain or are composed entirely of stable nucleic acid lipid particles (SNALPs). SNALPs may comprise an ionizable lipid (DLinDMA) (e.g., cationic at low pH), a neutral helper lipid, cholesterol, a diffusible polyethylene glycol (PEG)-lipid, or any combination thereof. In some examples, SNALPs may comprise synthetic cholesterol, dipalmitoylphosphatidylcholine, 3-N-[(w-methoxy polyethylene glycol) 2000) carbamoyl]-1,2-dimyrestyloxypropylamine, and cationic 1,2-dilinoleyloxy-3-N,Ndimethylaminopropane. In some examples, SNALPs may comprise synthetic cholesterol, 1,2-distearoyl-sn-glycero-3-phosphocholine, PEG-CDMA, and 1,2-dilinoleyloxy-3-(N;N-dimethyl) aminopropane (DLinDMAo).

Other non-limiting, exemplary SNALPs that can be used to deliver the cargos described herein can be any such SNALPs as described in Morrissey et al., Nature Biotechnology, Vol. 23, No. 8, August 2005, Zimmerman et al., Nature Letters, Vol. 441, 4 May 2006; Geisbert et al., Lancet 2010; 375:1896-905; Judge, J. Clin. Invest. 119:661-673 (2009); and Semple et al., Nature Niotechnology, Volume 28 Number 2 Feb. 2010, pp. 172-177.

### Other Lipids

The lipid particles may also comprise one or more other types of lipids, e.g., cationic lipids, such as amino lipid 2,2-dilinoleyl-4-dimethylaminoethyl-[1,3]-dioxolane (DLin-KC2-DMA), DLin-KC2-DMA4, C12-200 and colipids disteroylphosphatidyl choline, cholesterol, and PEG-DMG.

In some embodiments, the delivery vehicle can be or include a lipidoid, such as any of those set forth in, for example, US20110293703.

In some embodiments, the delivery vehicle can be or include an amino lipid, such as any of those set forth in, for example, Jayaraman, Angew. Chem. Int. Ed. 2012, 51, 8529-8533.

In some embodiments, the delivery vehicle can be or include a lipid envelope, such as any of those set forth in, for example, Korman et al., 2011. Nat. Biotech. 29:154-157.

### Lipoplexes Polyplexes

In some embodiments, the delivery vehicles contain or be composed entirely of lipoplexes and/or polyplexes. Lipoplexes may bind to negatively charged cell membrane and induce endocytosis into the cells. Examples of lipoplexes may be complexes comprising lipid(s) and non-lipid components. Examples of lipoplexes and polyplexes include FuGENE-6 reagent, a non-liposomal solution containing lipids and other components, zwitterionic amino lipids (ZALs), Ca2p (e.g., forming DNA/Ca²⁺ microcomplexes), polyethenimine (PEI) (e.g., branched PEI), and poly(L-lysine) (PLL).

### Sugar-Based Particles

In some embodiments, the delivery vehicle can be a sugar-based particle. In some embodiments, the sugar-based particles can be or include GalNAc, such as any of those described in WO2014118272; US20020150626; Nair, J K et al., 2014, Journal of the American Chemical Society 136 (49), 16958-16961; Østergaard et al., Bioconjugate Chem., 2015, 26 (8), pp 1451-1455.

### Cell Penetrating Peptides

In some embodiments, the delivery vehicles contain or are composed entirely of cell penetrating peptides (CPPs). CPPs are short peptides that facilitate cellular uptake of various molecular cargo (e.g., from nanosized particles to small chemical molecules and large fragments of DNA).

CPPs may be of different sizes, amino acid sequences, and charges. In some examples, CPPs can translocate the plasma membrane and facilitate the delivery of various molecular cargoes to the cytoplasm or an organelle. CPPs may be introduced into cells via different mechanisms, e.g., direct penetration in the membrane, endocytosis-mediated entry, and translocation through the formation of a transitory structure.

CPPs may have an amino acid composition that either contains a high relative abundance of positively charged amino acids such as lysine or arginine or has sequences that contain an alternating pattern of polar/charged amino acids and non-polar, hydrophobic amino acids. These two types of structures are referred to as polycationic or amphipathic, respectively. A third class of CPPs are the hydrophobic peptides, containing only apolar residues, with low net charge or have hydrophobic amino acid groups that are crucial for cellular uptake. Another type of CPPs is the trans-activating transcriptional activator (Tat) from Human Immunodeficiency Virus 1 (HIV-1). Examples of CPPs include to Penetratin, Tat (48-60), Transportan, and (R-AhX-R4) (Ahx refers to aminohexanoyl), Kaposi fibroblast growth factor (FGF) signal peptide sequence, integrin β3 signal peptide sequence, polyarginine peptide Args sequence, Guanine rich-molecular transporters, and sweet arrow peptide. Examples of CPPs and related applications also include those described in U.S. Pat. No. 8,372,951.

CPPs can be used for in vitro and ex vivo work quite readily, and extensive optimization for each cargo and cell type is usually required. In some examples, CPPs may be covalently attached to the polypeptides of the present invention (e.g., a SCN1B mimetic peptide described herein) directly and delivered to cells. In some examples, a SCN1B mimetic peptide-CPP and another molecule such as a polynucleotide-CPP or another polypeptide-CPP are each delivered separately to multiple cells. CPPs may also be used to delivery RNPs or other complexes (e.g., dimers etc.) including peptides of the present invention.

CPPs may be used to deliver the compositions and systems to plants. In some examples, CPPs may be used to deliver the components to plant protoplasts, which are then regenerated to plant cells and further to plants.

### DNA Nanoclews

In some embodiments, the delivery vehicles contain or are composed entirely of DNA nanoclews. A DNA nanoclew refers to a sphere-like structure of DNA (e.g., with a shape of a ball of yarn). The nanoclew may be synthesized by rolling circle amplification with palindromic sequences that aide in the self-assembly of the structure. The sphere may then be loaded with a payload. An example of DNA nanoclew is described in Sun W et al, J Am Chem Soc. 2014 Oct. 22;136 (42): 14722-5; and Sun W et al, Angew Chem Int Ed Engl. 2015 Oct. 5;54 (41): 12029-33. DNA nanoclew may have a palindromic sequences to be partially complementary to the a polynucleotide encoding a SCN1B mimetic peptide of the present invention complex. A DNA nanoclew may be coated, e.g., coated with PEI to induce endosomal escape.

### Metal Nanoparticles

in some embodiments, the delivery vehicles contain or are composed entirely of metal nanoparticles. In some embodiments, the delivery vehicles contain or are composed entirely of gold nanoparticles (also referred to AuNPs or colloidal gold). Gold nanoparticles may form complex with cargos, e.g., polynucleotides or polypeptides of the present invention described herein. Gold nanoparticles may be coated, e.g., coated in a silicate and an endosomal disruptive polymer, PAsp (DET). Examples of gold nanoparticles include AuraSense Therapeutics' Spherical Nucleic Acid (SNA™) constructs, and those described in Mout R, et al. (2017). ACS Nano 11:2452-8; Lee K, et al. (2017). Nat Biomed Eng 1:889-901. Other metal nanoparticles can also be complexed with cargo(s). Such metal nanoparticles include, without limitation, tungsten, palladium, rhodium, platinum, and iridium particles. Other non-limiting, exemplary metal nanoparticles suitable for delivery vehicles are described in U.S. Pat. No. 20,100,129793.

### Top

In some embodiments, the delivery vehicles contain or are composed entirely of iTOP. iTOP refers to a combination of small molecules drives the highly efficient intracellular delivery of native proteins, independent of any transduction peptide. iTOP may be used for induced transduction by osmocytosis and propanebetaine, using NaCl-mediated hyperosmolality together with a transduction compound (propanebetaine) to trigger macropinocytotic uptake into cells of extracellular macromolecules. Examples of iTOP methods and reagents include those described in D′Astolfo DS, Pagliero R J, Pras A, et al. (2015). Cell 161:674-690.

### Polymer-based Particles

In some embodiments, the delivery vehicles contain or are composed entirely of polymer-based particles (e.g., nanoparticles). In some embodiments, the polymer-based particles may mimic a viral mechanism of membrane fusion. The polymer-based particles may be a synthetic copy of Influenza virus machinery and form transfection complexes with various types of nucleic acids ((siRNA, miRNA, plasmid DNA or shRNA, mRNA) that cells take up via the endocytosis pathway, a process that involves the formation of an acidic compartment. The low pH in late endosomes acts as a chemical switch that renders the particle surface hydrophobic and facilitates membrane crossing. Once in the cytosol, the particle releases its payload for cellular action. This Active Endosome Escape technology is safe and maximizes transfection efficiency as it is using a natural uptake pathway. In some embodiments, the polymer-based particles may comprise alkylated and carboxyalkylated branched polyethylenimine. In some examples, the polymer-based particles are VIROMER, e.g., VIROMER RNAi, VIROMER RED, VIROMER mRNA, VIROMER CRISPR. Example methods of delivering the systems and compositions herein include those described in Bawage S S et al., Synthetic mRNA expressed Cas13a mitigates RNA virus infections, www.biorxiv.org/content/10.1101/370460v1.full doi: doi.org/10.1101/370460, Viromer® RED, a powerful tool for transfection of keratinocytes. doi: 10.13140/RG.2.2.16993.61281, Viromer® Transfection-Factbook 2018: technology, product overview, users' data., doi: 10.13140/RG.2.2.23912.16642. Other exemplary and non-limiting polymeric particles suitable for delivery vehicles are described in US20170079916, US20160367686, U.S. Pat. No. 20,110,212179, US20130302401, 6,007,845, 5,855,913, 5,985,309, 5,543,158, WO2012135025, US20130252281, US20130245107, US20130244279; US20050019923, 20080267903.

### Streptolysin O (SLO)

The delivery vehicles can contain or be composed entirely of streptolysin O (SLO). SLO is a toxin produced by Group A streptococci that works by creating pores in mammalian cell membranes. SLO may act in a reversible manner, which allows for the delivery of proteins (e.g., up to 100 kDa) to the cytosol of cells without compromising overall viability. Examples of SLO include those described in Sierig G, et al. (2003). Infect Immun 71:446-55; Walev I, et al. (2001). Proc Natl Acad Sci USA 98:3185-90; Teng K W, et al. (2017). Elife 6: e25460.

### Multifunctional Envelope-Type Nanodevice (MEND)

The delivery vehicles can contain or be composed entirely of multifunctional envelope-type nanodevice (MENDs). MENDs may comprise condensed plasmid DNA, a PLL core, and a lipid film shell. A MEND may further comprise cell-penetrating peptide (e.g., stearyl octaarginine). The cell penetrating peptide may be in the lipid shell. The lipid envelope may be modified with one or more functional components, e.g., one or more of: polyethylene glycol (e.g., to increase vascular circulation time), ligands for targeting of specific tissues/cells, additional cell-penetrating peptides (e.g., for greater cellular delivery), lipids to enhance endosomal escape, and nuclear delivery tags. In some examples, the MEND may be a tetra-lamellar MEND (T-MEND), which may target the cellular nucleus and mitochondria. In certain examples, a MEND may be a PEG-peptide-DOPE-conjugated MEND (PPD-MEND), which may target bladder cancer cells. Examples of MENDs include those described in Kogure K, et al. (2004). J Control Release 98:317-23; Nakamura T, et al. (2012). Acc Chem Res 45:1113-21.

### Lipid-Coated Mesoporous Silica Particles

The delivery vehicles can contain or be composed entirely of lipid-coated mesoporous silica particles. Lipid-coated mesoporous silica particles may comprise a mesoporous silica nanoparticle core and a lipid membrane shell. The silica core may have a large internal surface area, leading to high cargo loading capacities. In some embodiments, pore sizes, pore chemistry, and overall particle sizes may be modified for loading different types of cargos. The lipid coating of the particle may also be modified to maximize cargo loading, increase circulation times, and provide precise targeting and cargo release. Examples of lipid-coated mesoporous silica particles include those described in Du X, et al. (2014). Biomaterials 35:5580-90; Durfee P N, et al. (2016). ACS Nano 10:8325-45.

### Inorganic Nanoparticles

The delivery vehicles can contain or be composed entirely of inorganic nanoparticles. Examples of inorganic nanoparticles include carbon nanotubes (CNTs) (e.g., as described in Bates K and Kostarelos K. (2013). Adv Drug Deliv Rev 65:2023-33.), bare mesoporous silica nanoparticles (MSNPs) (e.g., as described in Luo G F, et al. (2014). Sci Rep 4:6064), and dense silica nanoparticles (SiNPs) (as described in Luo D and Saltzman W M. (2000). Nat Biotechnol 18:893-5).

### Exosomes

The delivery vehicles can contain or be composed entirely of exosomes. Exosomes include membrane bound extracellular vesicles, which can be used to contain and delivery various types of biomolecules, such as proteins, carbohydrates, lipids, and nucleic acids, and complexes thereof (e.g., RNPs). Examples of exosomes include those described in Schroeder A, et al., J Intern Med. 2010 January;267 (1): 9-21; El-Andaloussi S, et al., Nat Protoc. 2012 December;7 (12): 2112-26; Uno Y, et al., Hum Gene Ther. 2011 June;22 (6): 711-9; Zou W, et al., Hum Gene Ther. 2011 April;22 (4): 465-75.

In some examples, the exosome forms a complex (e.g., by binding directly or indirectly) to one or more components of the cargo. In certain examples, a molecule of an exosome may be fused with first adapter protein and a component of the cargo may be fused with a second adapter protein. The first and the second adapter protein may specifically bind each other, thus associating the cargo with the exosome. Examples of such exosomes include those described in Ye Y, et al., Biomater Sci. 2020 Apr. 28. doi: 10.1039/d0bm00427h.

Other non-limiting, exemplary exosomes include any of those set forth in Alvarez-Erviti et al. 2011, Nat Biotechnol 29:341; El-Andaloussi et al. (Nature Protocols 7:2112-2126 (2012); and Wahlgren et al. (Nucleic Acids Research, 2012, Vol. 40, No. 17 e130).

In some embodiments, the exosomes are as described in WO 2020/028439.

In some embodiments, the exosomes are milk derived exosomes. In some embodiments, the milk derived exosomes are isolated using a procedure detailed in International Patent Application No. PCT/US2022/017554.

### Spherical Nucleic Acids (SNAs)

Spherical nucleic acids (SNA) are three-dimensional arrangements of nucleic acids, with densely packed and radially arranged oligonucleotides on a central nanoparticle core. In its simplest form the SNA is composed of oligonucleotides and a core. In some embodiments, the delivery vehicle can contain or be composed entirely of SNAs. SNAs are three dimensional nanostructures that can be composed of densely functionalized and highly oriented nucleic acids that can be covalently attached to the surface of spherical nanoparticle cores. The core may be a hollow core which is produced by a 3-dimensional arrangement of molecules which form the outer boundary of the core. For instance, the molecules may be in the form of a lipid layer or bilayer which has a hollow center. In other embodiments, the molecules may be in the form of lipids, such as amphipathic lipids, i.e., sterols which are linked to an end the oligonucleotide. Sterols such as cholesterol linked to an end of an oligonucleotide may associate with one another and form the outer edge of a hollow core with the oligonucleotides radiating outward from the core. The core may also be a solid or semi-solid core.

The oligonucleotides to be delivered can be associated with the core of an SNP. An oligonucleotide that is associated with the core may be covalently linked to the core or non-covalently linked to the core, i.e., potentially through hydrophobic interactions. For instance, when a sterol forms the outer edge of the core an oligonucleotide may be covalently linked to the sterol directly or indirectly. When a lipid layer forms the core, the oligonucleotide may be covalently linked to the lipid or may be non-covalently linked to the lipids e.g., by interactions with the oligonucleotide or a molecule such as a cholesterol attached to the oligonucleotide directly or indirectly through a linker.

A spherical nucleic acid (SNA) can be functionalized in order to attach a polynucleotide. Alternatively or additionally, the polynucleotide can be functionalized. One mechanism for functionalization is the alkanethiol method, whereby oligonucleotides are functionalized with alkanethiols at their 3′ or 5′ termini prior to attachment to gold nanoparticles or nanoparticles comprising other metals, semiconductors or magnetic materials. Such methods are described, for example Whitesides, Proceedings of the Robert A. Welch Foundation 39^{th} Conference On Chemical Research Nanophase Chemistry, Houston, Tex., pages 109-121 (1995), and Mucic et al. Chem. Commun. 555-557 (1996). Oligonucleotides can also be attached to nanoparticles using other functional groups such as phosophorothioate groups, as described in and incorporated by reference from U.S. Pat. No. 5,472,881, or substituted alkylsiloxanes, as described in and incorporated by reference from Burwell, Chemical Technology, 4, 370-377 (1974) and Matteucci and Caruthers, J. Am. Chem. Soc., 103, 3185-3191 (1981). In some instances, oligonucleotides are attached to nanoparticles by terminating the polynucleotide with a 5′ or 3′ thionucleoside. In other instances, an aging process is used to attach oligonucleotides to nanoparticles as described in and incorporated by reference from U.S. Pat. Nos. 6,361,944, 6,506,569, 6,767,702 and 6,750,016 and PCT Publication Nos. WO 1998/004740, WO 2001/000876, WO 2001/051665 and WO 2001/073123. In some embodiments, the core is a metal core. In some embodiments, the core is an inorganic metal core. In some embodiments, the core is a gold core.

In some instances, the oligonucleotide is attached or inserted in the SNA. A spacer sequence can be included between the attachment site and the oligonucleotide. In some embodiments, a spacer sequence comprises or consists of an oligonucleotide, a peptide, a polymer or an oligoethylene glycol. In a preferred embodiment, the spacer is oligoethylene glycol and more preferably, hexaethyleneglycol.

Non-limiting, exemplary SNAs can be any of those set forth in Cutler et al., J. Am. Chem. Soc. 2011 133:9254-9257, Hao et al., Small. 2011 7:3158-3162, Zhang et al., ACS Nano. 2011 5:6962-6970, Cutler et al., J. Am. Chem. Soc. 2012 134:1376-1391, Young et al., Nano Lett. 2012 12:3867-71, Zheng et al., Proc. Natl. Acad. Sci. USA. 2012 109:11975-80, Mirkin, Nanomedicine 2012 7:635-638 Zhang et al., J. Am. Chem. Soc. 2012 134:16488-1691, Weintraub, Nature 2013 495: S14-S16, Choi et al., Proc. Natl. Acad. Sci. USA. 2013 110 (19): 7625-7630, Jensen et al., Sci. Transl. Med. 5, 209ra152 (2013) and Mirkin, et al., U.S. Pat. App. Pub. US20210002640 and US20200188521.

### Self-Assembling Nanoparticles

In some embodiments, the delivery vehicle contains or is composed entirely of a self-assembling nanoparticle. The self-assembling nanoparticles can contain one or more polymers. The self-assembling nanoparticles can be PEGylated. Self-assembling nanoparticles are known in the art. Non-limiting, exemplary self-assembling nanoparticles can any as set forth in Schiffelers et al., Nucleic Acids Research, 2004, Vol. 32, No. 19, Bartlett et al. (PNAS, Sep. 25, 2007, vol. 104, no. 39; Davis et al., Nature, Vol 464, 15 Apr. 2010.

### Supercharged Proteins

In some embodiments, the delivery vehicle contains or is composed entirely of supercharged protein. As used herein “Supercharged proteins” are a class of engineered or naturally occurring proteins with unusually high positive or negative net theoretical charge. Non-limiting, exemplary supercharged proteins can be any of those set forth in Lawrence et al., 2007, Journal of the American Chemical Society 129, 10110-10112.

### Targeted Delivery

In some embodiments, the delivery vehicle is configured for targeted delivery of cargo(s) (e.g., (e.g., the SCN1B mimetic polypeptides, SCN1B encoding polynucleotides, and/or other polypeptides and/or polynucleotides, vectors and/or the like of the present disclosure)) to a specific cell, tissue, organ, or system. In such embodiments, the delivery vehicle can include one or more targeting moieties that can direct targeted delivery of the cargo(s). In an embodiment, the delivery vehicle comprises a targeting moiety, such as on its surface. Exemplary targeting moieties include, without limitation, small molecule, polypeptide, and/or polynucleotide ligands for cell surface molecules, antibodies, affibodies, aptamers, or any combination thereof. In some embodiments, a targeted delivery vehicle can be generated by coupling, conjugating, attaching, or otherwise associating a targeting moiety with a delivery vehicle described elsewhere herein. In some embodiments, multiple targeting moieties with different targets are coupled to a delivery vehicle. In some embodiments a multivalent approach can be employed. Multivalent presentation of targeting moieties (e.g., antibodies) can also increase the uptake and signaling properties of targeting moiety fragments. In some embodiments, targeted delivery can be to one cell type or to multiple cell types. Methods of coupling conjugating, attaching, or otherwise associating a targeting moiety with a delivery vehicle are generally known in the art.

In some embodiments, the targeting moiety is an aptamer. Aptamers are ssDNA or RNA oligonucleotides that impart high affinity and specific recognition of the target molecules by electrostatic interactions, hydrogen bonding and hydrophobic interactions as opposed to the Watson-Crick base pairing, which is typical for the bonding interactions of oligonucleotides. Aptamers as a targeting moiety can have advantages over antibodies: aptamers can demonstrate higher target antigen recognition as compared with antibodies; aptamers can be more stable and smaller in size as compared with antibodies; aptamers can be easily synthesized and chemically modified for molecular conjugation; and aptamers can be changed in sequence for improved selectivity and can be developed to recognize poorly immunogenic targets.

Targeted delivery includes intracellular delivery. Delivery vehicles that utilize the endocytic pathway are entrapped in the endosomes (pH 6.5-6) and subsequently fuse with lysosomes (pH<5), where they undergo degradation that results in a lower therapeutic potential. The low endosomal pH can be taken advantage of to escape degradation. Fusogenic lipids or peptides, which destabilize the endosomal membrane after the conformational transition/activation at a lowered pH can be included in the delivery vehicle. Such lipids or peptides can include amines, which are protonated at an acidic pH and cause endosomal swelling and rupture by a buffer effect, pore-forming protein listeriolysin O, histidine-rich peptides have the ability to fuse with the endosomal membrane, resulting in pore formation, and can buffer the proton pump causing membrane lysis, and/or unsaturated dioleoylphosphatidylethanolamine (DOPE) that readily adopt an inverted hexagonal shape at a low pH and causes fusion of liposomes to the endosomal membrane. Inclusion of such molecules can result in an efficient endosomal release and/or may provide an endosomal escape mechanism to increase cargo delivery by the vehicle.

In some embodiments, the delivery vehicle is or includes modified CPP(s) that can facilitate intracellular delivery via macropinocytosis followed by endosomal escape. CPPs are described in greater detail elsewhere herein.

In some embodiments, targeted delivery is organelle-specific targeted delivery. A delivery vehicle can be surface-functionalized with a targeting moiety that can direct organelle specific delivery, such as a nuclear localization sequence, ribosomal entry sequence, mitochondria specific moiety, and/or the like. The invention further comprehends a lipid entity of the invention targeting the nucleus, e.g., via a DNA-intercalating moiety.

In some embodiments, the targeted delivery is multifunctional targeted delivery that can be accomplished by attaching more than one targeting moiety to the surface of the delivery vehicle. In some embodiments, such an enhances accumulation in a desired site and/or promotes organelle-specific delivery and/or target a particular type of cell and/or respond to the local environmental stimuli such as temperature (e.g., elevated), pH (e.g., acidic or basic), respond to targeted or localized externally applied stimuli such as a magnetic field, light, energy, heat or ultrasound (e.g., responsive delivery, which is described in greater detail elsewhere herein) and/or promote intracellular delivery of the cargo.

Exemplary targeting moieties are generally known in the art, and include without limitation, those described in e.g., in e.g., Deshpande et al, “Current trends in the use of liposomes for tumor targeting,” Nanomedicine (Lond). 8 (9), doi: 10.2217/nnm.13.118 (2013), International Patent Publication No. WO 2016/027264, Lorenzer et al, “Going beyond the liver: Progress and challenges of targeted delivery of siRNA therapeutics,” Journal of Controlled Release, 203:1-15 (2015); Surace et al, “Lipoplexes targeting the CD44 hyaluronic acid receptor for efficient transfection of breast cancer cells,” J. Mol Pharm 6 (4): 1062-73; doi: 10.1021/mp800215d (2009); Sonoke et al, “Galactose-modified cationic liposomes as a liver-targeting delivery system for small interfering RNA,” Biol Pharm Bull. 34 (8): 1338-42 (2011); Torchilin, “Antibody-modified liposomes for cancer chemotherapy,” Expert Opin. Drug Deliv. 5 (9), 1003-1025 (2008); Manjappa et al, “Antibody derivatization and conjugation strategies: application in preparation of stealth immunoliposome to target chemotherapeutics to tumor,” J. Control. Release 150 (1), 2-22 (2011); Sofou S “Antibody-targeted liposomes in cancer therapy and imaging,” Expert Opin. Drug Deliv. 5 (2): 189-204 (2008); Gao J et al, “Antibody-targeted immunoliposomes for cancer treatment,” Mini. Rev. Med. Chem. 13 (14): 2026-2035 (2013); Molavi et al, “Anti-CD30 antibody conjugated liposomal doxorubicin with significantly improved therapeutic efficacy against anaplastic large cell lymphoma,” Biomaterials 34 (34): 8718-25 (2013), Zhao et al., 2020. Cell 181:151-167, particularly at tables 1-5; Liu et al., Front. Bioeng. Biotechnol. 2021. 9:701504. doi: 10.3389/fbioe.2021.701504; US20210379192 (describes exemplary skeletal muscle cell targeting moieties), Snow-Lisy et al., Drug. Deliv. Transl. Res. 1: 351 (2011); US20060263336 (describes exemplary progenitor cell targeting moieties) each of which and the documents cited therein are hereby incorporated herein by reference.

Other exemplary targeting moieties are described elsewhere herein, such as epitope tags, reporter and selectable markers, and/or the like which can be configured for and/or operate in some embodiments as targeting moieties.

### Responsive Delivery

In some embodiments, the delivery vehicle can allow for responsive delivery of the cargo(s) (e.g., the SCN1B mimetic polypeptides, SCN1B encoding polynucleotides, and/or other polypeptides and/or polynucleotides, vectors and/or the like of the present disclosure). Responsive delivery, as used in this context herein, refers to delivery of cargo(s) by the delivery vehicle in response to an external stimuli. Examples of suitable stimuli include, without limitation, an energy (light, heat, cold, and the like), a chemical stimuli (e.g., chemical composition, etc.), and a biologic or physiologic stimuli (e.g., environmental pH, osmolarity, salinity, biologic molecule, etc.). In some embodiments, a targeting moiety is responsive to an external stimuli and facilitate responsive delivery. In other embodiments, responsiveness is determined by a non-targeting moiety component of the delivery vehicle.

In some embodiments, the responsive delivery is stimuli-sensitive, e.g., sensitive to an externally applied stimuli, such as magnetic fields, ultrasound or light; and pH-triggering can also be used, e.g., a labile linkage can be used between a hydrophilic moiety such as PEG and a hydrophobic moiety such as a lipid entity of the invention, which is cleaved only upon exposure to the relatively acidic conditions characteristic of the a particular environment or microenvironment such as an endocytic vacuole or the acidotic tumor mass. pH-sensitive copolymers can also be incorporated in embodiments of the invention can provide shielding; diortho esters, vinyl esters, cysteine-cleavable lipopolymers, double esters and hydrazones are a few examples of pH-sensitive bonds that are quite stable at pH 7.5, but are hydrolyzed relatively rapidly at pH 6 and below, e.g., a terminally alkylated copolymer of N-isopropylacrylamide and methacrylic acid that copolymer facilitates destabilization of a lipid entity of the invention and release in compartments with decreased pH value; or, the invention comprehends ionic polymers for generation of a pH-responsive lipid entity of the invention (e.g., poly(methacrylic acid), poly(diethylaminoethyl methacrylate), poly(acrylamide) and poly(acrylic acid)).

In some embodiments, the responsive delivery is temperature-triggered delivery. Many pathological areas, such as inflamed tissues and tumors, show a distinctive hyperthermia compared with normal tissues. Utilizing this hyperthermia is an attractive strategy in cancer therapy since hyperthermia is associated with increased tumor permeability and enhanced uptake. This technique involves local heating of the site to increase microvascular pore size and blood flow, which, in turn, can result in an increased extravasation of embodiments of the invention. Temperature-sensitive lipid entity of the invention can be prepared from thermosensitive lipids or polymers with a low critical solution temperature. Above the low critical solution temperature (e.g., at site such as tumor site or inflamed tissue site), the polymer precipitates, disrupting the liposomes to release. Lipids with a specific gel-to-liquid phase transition temperature are used to prepare these lipid entities of the invention; and a lipid for a thermosensitive embodiment can be dipalmitoylphosphatidylcholine. Thermosensitive polymers can also facilitate destabilization followed by release, and a useful thermosensitive polymer is poly(N-isopropylacrylamide). Another temperature triggered system can employ lysolipid temperature-sensitive liposomes.

In some embodiments, the responsive delivery is redox-triggered delivery. The difference in redox potential between normal and inflamed or tumor tissues, and between the intra- and extra-cellular environments has been exploited for delivery, e.g., GSH is a reducing agent abundant in cells, especially in the cytosol, mitochondria and nucleus. The GSH concentrations in blood and extracellular matrix are just one out of 100 to one out of 1000 of the intracellular concentration, respectively. This high redox potential difference caused by GSH, cysteine and other reducing agents can break the reducible bonds, destabilize a lipid entity of the invention and result in release of payload. The disulfide bond can be used as the cleavable/reversible linker in a lipid entity of the invention, because it causes sensitivity to redox owing to the disulfide-to-thiol reduction reaction; a lipid entity of the invention can be made reduction sensitive by using two (e.g., two forms of a disulfide-conjugated multifunctional lipid as cleavage of the disulfide bond (e.g., via tris(2-carboxyethyl) phosphine, dithiothreitol, L-cysteine or GSH), can cause removal of the hydrophilic head group of the conjugate and alter the membrane organization leading to release of payload. Calcein release from reduction-sensitive lipid entity of the invention containing a disulfide conjugate can be more useful than a reduction-insensitive embodiment.

Enzymes can also be used as a trigger to release payload. Enzymes, including MMPs (e.g. MMP2), phospholipase A2, alkaline phosphatase, transglutaminase or phosphatidylinositol-specific phospholipase C, have been found to be overexpressed in certain tissues, e.g., tumor tissues. In the presence of these enzymes, specially engineered enzyme-sensitive lipid entity of the invention can be disrupted and release the payload. an MMP2-cleavable octapeptide (Gly-Pro-Leu-Gly-Ile-Ala-Gly-Gln (SEQ ID NO: 46) can be incorporated into a linker, and can have antibody targeting, e.g., antibody 2C5.

In some embodiments, the responsive delivery is light- or energy-triggered delivery, e.g., the lipid entity of the invention can be light-sensitive, such that light or energy can facilitate structural and conformational changes, which lead to direct interaction of the lipid entity of the invention with the target cells via membrane fusion, photo-isomerism, photofragmentation or photopolymerization; such a moiety therefor can be benzoporphyrin photosensitizer. Ultrasound can be a form of energy to trigger delivery; a lipid entity of the invention with a small quantity of particular gas, including air or perfluorated hydrocarbon can be triggered to release with ultrasound, e.g., low-frequency ultrasound (LFUS). Magnetic delivery: A lipid entity of the invention can be magnetized by incorporation of magnetites, such as Fe304 or γ-Fe203, e.g., those that are less than 10 nm in size. Targeted delivery can be then by exposure to a magnetic field.

Responsive delivery to the testis has been described. See e.g., He et al., 2015. Oncol. Rep. 34 (5)-2318 (describes ultrasound microbubble-mediated delivery to the testis); Li et al., Curr. Drug. Deliv. 2020 17 (5): 438-446 (describes heat stress and pulsed unfocused ultrasound delivery into testicular seminiferous tubules), which can be adapted for use with the present disclosure to provide responsive delivery to the testis or testicular cell.

### Cells and Organisms

Described herein are engineered cells that are engineered to express and/or secrete one or more of the SCN1B mimetic peptides described herein. In some embodiments, engineered cells can be administered as part of a therapy to a subject in need thereof. In some embodiments, the engineered cells are used to produce SCN1B mimetic peptides described herein for subsequent harvesting and administration. In some embodiments, the engineered cells are part of an in vitro production system for producing the SCN1B mimetic peptides. In some embodiments, engineered non-human animals or plants are generated that express the SCN1B mimetic peptides, such as in a biological fluid or harvestable plant part, are used as bioreactors for the production of the SCN1B mimetic peptides.

In general, an SCN1B mimetic peptide encoding polynucleotide is expressed, such as via a vector or vector system, or is otherwise integrated into the genome of the cell, such that the SCN1B mimetic peptides are expressed in the cell and optionally secreted. Methods and techniques of modifying animal and plant cells are generally known in the art and can be used to engineer the cells to express and optionally secrete the SCN1B mimetic peptides of the present disclosure.

### Cells for Cell-Based Therapies

In some embodiments, engineered cells can be administered as part of a therapy to a subject in need thereof. In some embodiments, the cells are allogeneic to a subject in which they are delivered. In some embodiments, the cells are autologous to a subject in which they are delivered. In some embodiments, the cells are engineered ex vivo to express and/or secrete one or more of the SCN1B mimetic peptides described herein prior to administration to a subject in need thereof. The cells can provide production of the SCN1B mimetic peptides described herein within the subject in need thereof.

The use of autologous cells can minimize graft-versus-host disease (GVHD) issues. In some embodiments, allogenic cells are modified to reduce alloreactivity and prevent graft-versus-host disease. This approach can have the advantage as being able to provide “off the shelf” solutions and cells for cell-based therapies and delivery of the SCN1B mimetic peptides and provide a scalable alternative to autologous approaches. Platforms for manufacturing allogenic and autologous cells (including modificed cells) are generally known in the art. See e.g., Abbasalizadeh Expert Opin Biol Ther. 2017 October; 17 (10): 1201-1219; Pigeau et al., Front Med (Lausanne). 2018 Aug. 22; 5:233; Abraham et al., Adv Biochem Eng Biotechnol. 2018; 165:323-350. Exemplary modifications that can be introduced allogenic cells to reduce complications to alloreactivity are known in the art. See e.g., Perez et al., Front Immunol. 2020 Nov. 11; 11:583716. doi: 10.3389.

In some embodiments, the engineered cells for delivery of the SCN1B mimetic peptides can be engineered to include a transgenic safety switch, in the form of a transgene that renders the cells vulnerable to exposure to a specific signal. For example, in some embodiments, the cells can be engineered with a thymidine kinase (TK) gene that, in response to a nucleoside prodrug (e.g., ganciclovir or acyclovir), causes cell death (see e.g., Greco, et al., Improving the safety of cell therapy with the TK-suicide gene. Front. Pharmacol. 2015; 6:95). Alternative safety switch constructs include inducible caspase 9, for example triggered by administration of a small-molecule dimerizer that brings together two nonfunctional icasp9 molecules to form the active enzyme. A wide variety of alternative approaches to implementing cellular proliferation controls have been described (see U.S. Patent Publication No. 20130071414; PCT Patent Publication WO2011146862; PCT Patent Publication WO2014011987; PCT Patent Publication WO2013040371; Zhou et al. BLOOD, 2014, 123/25:3895-3905; Di Stasi et al., The New England Journal of Medicine 2011; 365:1673-1683; Sadelain M, The New England Journal of Medicine 2011; 365:1735-173; Ramos et al., Stem Cells 28 (6): 1107-15 (2010)).

In the instance of allogenic cells, the allogenic cells can be engineered to be resistant to immunosuppressive agents (e.g., calcineurin inhibitor, a target of rapamycin, an interleukin-2 receptor α-chain blocker, an inhibitor of inosine monophosphate dehydrogenase, an inhibitor of dihydrofolic acid reductase, a corticosteroid or an immunosuppressive antimetabolite), which are often given to subjects receiving an allogenic therapy. In some embodiments, immunosuppressive resistance is conferred to the allogeneic cells by inactivating the target of the immunosuppressive agent in T cells. As non-limiting examples, targets for an immunosuppressive agent can be a receptor for an immunosuppressive agent such as: CD52, glucocorticoid receptor (GR), a FKBP family gene member and a cyclophilin family gene member.

The engineered cells can be any suitable cell type. The engineered cells can be cultured and expanded as desired prior to delivery to the subject. Collection, isolation, selection, culture, expansion, and storage methods for the engineered cells are generally known in the art.

### Engineered Cells and Organisms for SCN1B Mimetic Peptide Production

In some embodiments, the cells can be engineered to express and produce SCN1B mimetic peptides described herein for subsequent harvesting and delivery to a subject in need thereof. Various cell and organism based bioreactors for recombinant protein production are generally known in the art and can be employed for production of the SCN1B mimetic peptides of the present disclosure. Exemplary cells and organism are discussed below.

### Plants

In some embodiments, the engineered organism is a plant or algae. Plants have been engineered for the production of therapeutics, such as therapeutic proteins. Plant-based biofactories are an established production system with the benefits of cost-effectiveness, high scalability, rapid production, enabling post-translational modification, and being devoid of harmful pathogens contamination. See e.g., Sethi et al., Mol Biotechnol. 2021 December;63 (12): 1125-1137; Yusibov et al., Annu Rev Plant Biol. 2016 Apr. 29; 67:669-701; Huang and McDonald. Biotechnol Adv. 2012 March-Apr;30 (2): 398-409; Yang et al., Biomolecules. 2021 Jan. 13;11 (1): 93; Vitale et al., Mol Interv. 2005 August;5 (4): 216-25. doi: 10.1124/mi.5.4.5; Singh et al., Curr Mol Biol Rep. 2017;3 (4): 306-316; Decker et al., Curr Opin Plant Biol. 2004 April; 7 (2): 166-70; Fischer et al., Curr Opin Plant Biol. 2004 April;7 (2): 152-8; Yemets et al., Cell Biol Int. 2014 September;38 (9): 989-1002; Daniell et al., Trends Plant Sci. 2009 December; 14 (12): 669-79, which are incorporated herein by reference and can be adapted for use with the present disclosure.

The engineered plants (and plant cells) include, but are not limited to, monocotyledonous and dicotyledonous plants (and plant cells), such as crops including grain crops (e.g., wheat, maize, rice, millet, barley), fruit crops (e.g., tomato, apple, pear, strawberry, orange), forage crops (e.g., alfalfa), root vegetable crops (e.g., carrot, potato, sugar beets, yam), leafy vegetable crops (e.g., lettuce, spinach); flowering plants (e.g., petunia, rose, chrysanthemum), conifers and pine trees (e.g., pine fir, spruce); plants used in phytoremediation (e.g., heavy metal accumulating plants); oil crops (e.g., sunflower, rape seed) and plants used for experimental purposes (e.g., Arabidopsis). Engineered plant cells and tissues include, without limitation, roots, stems, leaves, flowers, and reproductive structures, undifferentiated meristematic cells, parenchyma, collenchyma, sclerenchyma, xylem, phloem, epidermis, and germplasm. In some embodiments, the dicotyledonous plants belong to the order Magniolales, Illiciales, Laurales, Piperales, Aristochiales, Nymphaeales, Ranunculales, Papeverales, Sarraceniaceae, Trochodendrales, Hamamelidales, Eucomiales, Leitneriales, Myricales, Fagales, Casuarinales, Caryophyllales, Batales, Polygonales, Plumbaginales, Dilleniales, Theales, Malvales, Urticales, Lecythidales, Violales, Salicales, Capparales, Ericales, Diapensales, Ebenales, Primulales, Rosales, Fabales, Podostemales, Haloragales, Myrtales, Cornales, Proteales, San tales, Rafflesiales, Celastrales, Euphorbiales, Rhamnales, Sapindales, Juglandales, Geraniales, Polygalales, Umbellales, Gentianales, Polemoniales, Lamiales, Plantaginales, Scrophulariales, Campanulales, Rubiales, Dipsacales, or Asterales. In some embodiments, the monocotyledonous plants (or plant cells) belong to the order Alismatales, Hydrocharitales, Najadales, Triuridales, Commelinales, Eriocaulales, Restionales, Poales, Juncales, Cyperales, Typhales, Bromeliales, Zingiberales, Arecales, Cyclanthales, Pandanales, Arales, Lilliales, and Orchid ales, or belonging to Gymnospermae, e.g those belonging to the orders Pinales, Ginkgoales, Cycadales, Araucariales, Cupressales and Gnetales.

In some embodiments, the engineered plant is a dicot, monocot, or gymnosperm genera hereunder: Atropa, Alseodaphne, Anacardium, Arachis, Beilschmiedia, Brassica, Carthamus, Cocculus, Croton, Cucumis, Citrus, Citrullus, Capsicum, Catharanthus, Cocos, Coffea, Cucurbita, Daucus, Duguetia, Eschscholzia, Ficus, Fragaria, Glaucium, Glycine, Gossypium, Helianthus, Hevea, Hyoscyamus, Lactuca, Landolphia, Linum, Litsea, Lycopersicon, Lupinus, Manihot, Majorana, Malus, Medicago, Nicotiana, Olea, Parthenium, Papaver, Persea, Phaseolus, Pistacia, Pisum, Pyrus, Prunus, Raphanus, Ricinus, Senecio, Sinomenium, Stephania, Sinapis, Solanum, Theobroma, Trifolium, Trigonella, Vicia, Vinca, Vilis, and Vigna; and the genera Allium, Andropogon, Aragrostis, Asparagus, Avena, Cynodon, Elaeis, Festuca, Festulolium, Heterocallis, Hordeum, Lemna, Lolium, Musa, Oryza, Panicum, Pannesetum, Phleum, Poa, Secale, Sorghum, Triticum, Zea, Abies, Cunninghamia, Ephedra, Picea, Pinus, or Pseudotsuga.

In some embodiments the engineered cells are “algae” or “algae cells”; including for example algae selected from several eukaryotic phyla, including the Rhodophyta (red algae), Chlorophyta (green algae), Phaeophyta (brown algae), Bacillariophyta (diatoms), Eustigmatophyta and dinoflagellates as well as the prokaryotic phylum Cyanobacteria (blue-green algae). The term “algae” includes for example algae selected from: Amphora, Anabaena, Anikstrodesmis, Botryococcus, Chaetoceros, Chlamydomonas, Chlorella, Chlorococcum, Cyclotella, Cylindrotheca, Dunaliella, Emiliana, Euglena, Hematococcus, Isochrysis, Monochrysis, Monoraphidium, Nannochloris, Nannnochloropsis, Navicula, Nephrochloris, Nephroselmis, Nitzschia, Nodularia, Nostoc, Oochromonas, Oocystis, Oscillartoria, Pavlova, Phaeodactylum, Playtmonas, Pleurochrysis, Porhyra, Pseudoanabaena, Pyramimonas, Stichococcus, Synechococcus, Synechocystis, Tetraselmis, Thalassiosira, and Trichodesmium.

Also encompassed herein are gametes, seeds, germplasm, embryos, either zygotic or somatic, progeny or hybrids of plants comprising the recombinant SCN1B peptides and/or encoding polynucleotides, which are produced by traditional breeding methods.

### Bacterial Cells

In some embodiments, the engineered cells are bacterial cells. Suitable bacterial cell systems for recombinant protein production are generally known in the art See e.g., Baeshen et al., Microb Cell Fact. 2014 Oct. 2; 13:141, Mergulhão et al., Biotechnol Adv. 2005 May;23 (3): 177-202. doi: 10.1016/j.biotechadv.2004.11.003; Sørensen and Mortensen. J Biotechnol. 2005 Jan. 26;115 (2): 113-28; Porowińska et al., Postepy Hig Med Dosw (Online). 2013 Mar. 1; 67:119-29, which are incorporated herein by reference and can be adapted for use with the present disclosure.

### Fungal and Insect Cells

In some embodiments, the engineered cells are fungal or insect cells. Suitable fungal and insect cell systems for recombinant protein production are generally known in the art. See e.g., Mattanovich et al., Methods Mol Biol. 2012; 824:329-58, Ahmad et al., et al., Appl Microbiol Biotechnol. 2014 June;98 (12): 5301-17; Vandermies and Fickers. Microorganisms. 2019 Jan. 30;7 (2): 40; Ferrer-Miralles et al., Methods Mol Biol. 2015; 1258:1-24; Yang and Zhang. Biotechnol Adv. 2018 January-Feb;36 (1): 182-195; Karbalaei et al., J Cell Physiol. 2020 Sep;235 (9): 5867-5881; Baghban et al., Mol Biotechnol. 2019 May;61 (5): 365-384; Manfrão-Netto et al., Front Bioeng Biotechnol. 2019 May 1; 7:94; Baeshen et al., Microb Cell Fact. 2014 Oct. 2; 13:141, Chambers et al., Curr Protoc Protein Sci. 2018 Feb. 21; 91:5.4.1-5.4.6; Irons et al., Curr Protoc Protein Sci. 2018 Feb. 21; 91:5.5.1-5.5.22, which are incorporated herein by reference and can be adapted for use with the present disclosure.

### Non-Human Animals

In some embodiments, the engineered cells are non-human animal cells, optionally non-human mammalian cells. Suitable mammalian cells and systems for recombinant protein expression and production are generally known in the art. See e.g., O'Flaherty R. Biotechnol Adv. 2020 Nov. 1; 43:107552; Wurm. Nat Biotechnol. 2004 November;22 (11): 1393-8; Li et al., MAbs. 2010 September-Oct;2 (5): 466-79; Dyson et al., Adv Exp Med Biol. 2016; 896:217-24; Panavas et al., Methods Mol Biol. 2009; 497:303-17; Chevallier et al., Biotechnol Bioeng. 2020 April; 117 (4): 1172-1186; Dumont et al., Crit Rev Biotechnol. 2016 December;36 (6): 1110-1122; Hacker and Balasubramanian. Curr Opin Struct Biol. 2016 June;38:129-36; Bielser et al., Biotechnol Adv. 2018 July-Aug;36 (4): 1328-1340; Lalonde and Durocher et al., J Biotechnol. 2017 Jun. 10; 251:128-140, which are incorporated herein by reference and can be adapted for use with the present disclosure.

In some embodiments, the engineered organism is a non-human animal, such as a swine, bovine, ovine, caprine, camelid, equine, avian and/or the like. In some embodiments, a non-human animal bioreactor is used to produce the SCN1B mimetic peptides. See e.g., Houdebine et al., Transgenic Res. 2000;9 (4-5): 305-20; Demain and Vaishnav. Biotechnol Adv. 2009 May-Jun;27 (3): 297-306; Woodfint et al., Mol Biotechnol. 2018 December;60 (12): 975-983; Lubon et al., Transfus Med Rev. 1996 April; 10 (2): 131-43; Redwan el-RM. J Immunoassay Immunochem. 2009; 30 (3): 262-90; Lubon, H. Biotechnol Annu Rev. 1998; 4:1-54; Bertolini et al., Transgenic Res. 2016 June;25 (3): 329-43; Monzani et al., Bioengineered. 2016 April; 7 (3): 123-31; Ivarie et al., ends Biotechnol. 2006 March;24 (3): 99-101; Lillico et al., Drug Discov Today. 2005 Feb. 1;10 (3): 191-6; Dyck et al., Trends Biotechnol. 2003 September;21 (9): 394-9; Sid and Schusser et al 2018. Front. Genet. Doi.org/10.3389/fgene.2018.00456; Scott et al. 2010. ILAR J. 51 (4): 353-361; Yum et al., 2016. Scientific Reports. 6:27185; Tait-Burkard et al. 2018. Genome Biology. 19:2014; Kalds et al., 2019. Front. Genet. Doi.org//10.3389/fgene.2019.00750; Hryhorowicz et al., Genes (Basel). 2020 June 19;11 (6): 670; Monzani et al., Bioengineered. 2016 April;7 (3): 123-31, which are incorporated herein by reference and can be adapted for use with the present disclosure.

### Pharmaceutical Formulations

Also described herein are pharmaceutical formulations that can contain an amount, effective amount, and/or least effective amount, and/or therapeutically effective amount of one or more compounds, molecules, compositions, vectors, vector systems, cells, or a combination thereof (which are also referred to as the primary active agent or ingredient elsewhere herein) described in greater detail elsewhere herein and a pharmaceutically acceptable carrier or excipient. As used herein, “pharmaceutical formulation” refers to the combination of an active agent, compound, or ingredient with a pharmaceutically acceptable carrier or excipient, making the composition suitable for diagnostic, therapeutic, or preventive use in vitro, in vivo, or ex vivo. As used herein, “pharmaceutically acceptable carrier or excipient” refers to a carrier or excipient that is useful in preparing a pharmaceutical formulation that is generally safe, non-toxic, and is neither biologically or otherwise undesirable, and includes a carrier or excipient that is acceptable for veterinary use as well as human pharmaceutical use. A “pharmaceutically acceptable carrier or excipient” as used in the specification and claims includes both one and more than one such carrier or excipient. When present, the compound can optionally be present in the pharmaceutical formulation as a pharmaceutically acceptable salt. In some embodiments, the pharmaceutical formulation can include, such as an active ingredient, one or more of the SCN1B mimetic polypeptides and/or SCN1B encoding polynucleotides of the present disclosure.

In some embodiments, the active ingredient is present as a pharmaceutically acceptable salt of the active ingredient. As used herein, “pharmaceutically acceptable salt” refers to any acid or base addition salt whose counter-ions are non-toxic to the subject to which they are administered in pharmaceutical doses of the salts. Suitable salts include, hydrobromide, iodide, nitrate, bisulfate, phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, napthalenesulfonate, propionate, malonate, mandelate, malate, phthalate, and pamoate.

The pharmaceutical formulations described herein can be administered to a subject in need thereof via any suitable method or route to a subject in need thereof. Suitable administration routes can include, but are not limited to auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal (dental), intracoronary, intracorporus cavernosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratympanic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, subarachnoid, subconjunctival, subcutaneous, sublingual, submucosal, topical, transdermal, transmucosal, transplacental, transtracheal, transtympanic, ureteral, urethral, and/or vaginal administration, and/or any combination of the above administration routes, which typically depends on the disease to be treated and/or the active ingredient(s).

Where appropriate, compounds, molecules, compositions, vectors, vector systems, cells, or a combination thereof described in greater detail elsewhere herein can be provided to a subject in need thereof as an ingredient, such as an active ingredient or agent, in a pharmaceutical formulation. As such, also described are pharmaceutical formulations containing one or more of the compounds and salts thereof, or pharmaceutically acceptable salts thereof described herein. Suitable salts include, hydrobromide, iodide, nitrate, bisulfate, phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, napthalenesulfonate, propionate, malonate, mandelate, malate, phthalate, and pamoate.

In some embodiments, the subject in need thereof has or is suspected of having a hematopoietic disease or a symptom thereof. In some embodiments, the subject in need thereof has or is suspected of having, a neurobiological disease or disorder, a psychiatric disease or disorder, a cancer, an autoimmune disease or disorder, a thrombosis disease, a heart disease, a kidney disease, a lung disease, or a blood vessel disease, or a combination thereof. In some embodiments, the disease is an arrythmia. As used herein, “agent” refers to any substance, compound, molecule, and the like, which can be biologically active or otherwise can induce a biological and/or physiological effect on a subject to which it is administered to. As used herein, “active agent” or “active ingredient” refers to a substance, compound, or molecule, which is biologically active or otherwise, induces a biological or physiological effect on a subject to which it is administered to. In other words, “active agent” or “active ingredient” refers to a component or components of a composition to which the whole or part of the effect of the composition is attributed. An agent can be a primary active agent, or in other words, the component(s) of a composition to which the whole or part of the effect of the composition is attributed. An agent can be a secondary agent, or in other words, the component(s) of a composition to which an additional part and/or other effect of the composition is attributed.

### Pharmaceutically Acceptable Carriers and Secondary Ingredients and Agents

The pharmaceutical formulation can include a pharmaceutically acceptable carrier. Suitable pharmaceutically acceptable carriers include, but are not limited to water, salt solutions, alcohols, gum arabic, vegetable oils, benzyl alcohols, polyethylene glycols, gelatin, carbohydrates such as lactose, amylose or starch, magnesium stearate, talc, silicic acid, viscous paraffin, perfume oil, fatty acid esters, hydroxy methylcellulose, and polyvinyl pyrrolidone, which do not deleteriously react with the active composition.

The pharmaceutical formulations can be sterilized, and if desired, mixed with agents, such as lubricants, preservatives, stabilizers, wetting agents, emulsifiers, salts for influencing osmotic pressure, buffers, coloring, flavoring and/or aromatic substances, and the like which do not deleteriously react with the active compound.

In some embodiments, the pharmaceutical formulation can also include an effective amount of secondary active agents, including but not limited to, biologic agents or molecules including, but not limited to, e.g. polynucleotides, amino acids, peptides, polypeptides, antibodies, aptamers, ribozymes, hormones, immunomodulators, antipyretics, anxiolytics, antipsychotics, analgesics, antispasmodics, anti-inflammatoires, anti-histamines, anti-infectives, chemotherapeutics, and combinations thereof.

### Effective Amounts

In some embodiments, the amount of the primary active agent and/or optional secondary agent can be an effective amount, least effective amount, and/or therapeutically effective amount. As used herein, “effective amount” refers to the amount of the primary and/or optional secondary agent included in the pharmaceutical formulation that achieve one or more therapeutic effects or desired effect. As used herein, “least effective” amount refers to the lowest amount of the primary and/or optional secondary agent that achieves the one or more therapeutic or other desired effects. As used herein, “therapeutically effective amount” refers to the amount of the primary and/or optional secondary agent included in the pharmaceutical formulation that achieves one or more therapeutic effects. In some embodiments, the one or more therapeutic effects are reducing or altering an arrythmia.

The effective amount, least effective amount, and/or therapeutically effective amount of the primary and optional secondary active agent described elsewhere herein contained in the pharmaceutical formulation can be any non-zero amount ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pg, ng, ug, mg, or g or be any numerical value or subrange within any of these ranges.

In some embodiments, the effective amount, least effective amount, and/or therapeutically effective amount can be an effective concentration, least effective concentration, and/or therapeutically effective concentration, which can each be any non-zero amount ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pM, nM, uM, mM, or M or be any numerical value or subrange within any of these ranges.

In other embodiments, the effective amount, least effective amount, and/or therapeutically effective amount of the primary and optional secondary active agent be any non-zero amount ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 IU or be any numerical value or subrange within any of these ranges.

In some embodiments, the primary and/or the optional secondary active agent present in the pharmaceutical formulation can be any non-zero amount ranging from about 0 to 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.9, to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the pharmaceutical formulation or be any numerical value or subrange within any of these ranges.

In some embodiments where a cell or cell population is present in the pharmaceutical formulation (e.g., as a primary and/or or secondary active agent), the effective amount of cells can be any amount ranging from about 1 or 2 cells to 1×10¹/mL, 1×10²⁰/mL or more, such as about 1×10¹/mL, 1×10²/mL, 1×10³/mL, 1×10⁴/mL, 1×10⁵/mL, 1×10⁶/mL, 1×10⁷/mL, 1×10⁸/mL, 1×10⁹/mL, 1×10¹⁰/mL, 1×10¹¹/mL, 1×10¹²/mL, 1×10¹³/mL, 1×10¹⁴/mL, 1×10¹⁵/mL, 1×10¹⁶/mL, 1×10¹⁷/mL, 1×10¹⁸/mL, 1×10¹⁹/mL, to/or about 1×10²⁰/mL or any numerical value or subrange within any of these ranges.

In some embodiments, the amount or effective amount, particularly where an infective particle is being delivered (e.g., a virus particle having the primary or secondary agent as a cargo), the effective amount of virus particles can be expressed as a titer (plaque forming units per unit of volume) or as a MOI (multiplicity of infection). In some embodiments, the effective amount can be about 1×10¹ particles per pL, nL, μL, mL, or L to 1×10²⁰/particles per pL, nL, μL, mL, or L or more, such as about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, to/or about 1×10²⁰ particles per pL, nL, μL, mL, or L. In some embodiments, the effective titer can be about 1×10¹ transforming units per pL, nL, μL, mL, or L to 1×10²⁰/transforming units per pL, nL, μL, mL, or L or more, such as about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, to/or about 1×10²⁰ transforming units per pL, nL, μL, mL, or L or any numerical value or subrange within these ranges. In some embodiments, the MOI of the pharmaceutical formulation can range from about 0.1 to 10 or more, such as 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10 or more or any numerical value or subrange within these ranges.

In some embodiments, the amount or effective amount of the one or more of the active agent(s) described herein contained in the pharmaceutical formulation can range from about 1 μg/kg to about 10 mg/kg based upon the bodyweight of the subject in need thereof or average bodyweight of the specific patient population to which the pharmaceutical formulation can be administered.

In embodiments where there is a secondary agent contained in the pharmaceutical formulation, the effective amount of the secondary active agent will vary depending on the secondary agent, the primary agent, the administration route, subject age, disease, stage of disease, among other things, which will be one of ordinary skill in the art.

When optionally present in the pharmaceutical formulation, the secondary active agent can be included in the pharmaceutical formulation or can exist as a stand-alone compound or pharmaceutical formulation that can be administered contemporaneously or sequentially with the compound, derivative thereof, or pharmaceutical formulation thereof.

In some embodiments, the effective amount of the secondary active agent, when optionally present, is any non-zero amount ranging from about 0 to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the total active agents present in the pharmaceutical formulation or any numerical value or subrange within these ranges. In additional embodiments, the effective amount of the secondary active agent is any non-zero amount ranging from about 0 to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the total pharmaceutical formulation or any numerical value or subrange within these ranges.

### Dosage Forms

In some embodiments, the pharmaceutical formulations described herein can be provided in a dosage form. The dosage form can be administered to a subject in need thereof. The dosage form can be effective generate specific concentration, such as an effective concentration, at a given site in the subject in need thereof. As used herein, “dose,” “unit dose,” or “dosage” can refer to physically discrete units suitable for use in a subject, each unit containing a predetermined quantity of the primary active agent, and optionally present secondary active ingredient, and/or a pharmaceutical formulation thereof calculated to produce the desired response or responses in association with its administration. In some embodiments, the given site is proximal to the administration site. In some embodiments, the given site is distal to the administration site. In some cases, the dosage form contains a greater amount of one or more of the active ingredients present in the pharmaceutical formulation than the final intended amount needed to reach a specific region or location within the subject to account for loss of the active components such as via first and second pass metabolism.

The dosage forms can be adapted for administration by any appropriate route. Appropriate routes include, but are not limited to, oral (including buccal or sublingual), rectal, intraocular, inhaled, intranasal, topical (including buccal, sublingual, or transdermal), vaginal, parenteral, subcutaneous, intramuscular, intravenous, internasal, and intradermal. Other appropriate routes are described elsewhere herein. Such formulations can be prepared by any method known in the art.

Dosage forms adapted for oral administration can discrete dosage units such as capsules, pellets or tablets, powders or granules, solutions, or suspensions in aqueous or non-aqueous liquids; edible foams or whips, or in oil-in-water liquid emulsions or water-in-oil liquid emulsions. In some embodiments, the pharmaceutical formulations adapted for oral administration also include one or more agents which flavor, preserve, color, or help disperse the pharmaceutical formulation. Dosage forms prepared for oral administration can also be in the form of a liquid solution that can be delivered as a foam, spray, or liquid solution. The oral dosage form can be administered to a subject in need thereof. Where appropriate, the dosage forms described herein can be microencapsulated.

The dosage form can also be prepared to prolong or sustain the release of any ingredient. In some embodiments, compounds, molecules, compositions, vectors, vector systems, cells, or a combination thereof described herein can be the ingredient whose release is delayed. In some embodiments the primary active agent is the ingredient whose release is delayed. In some embodiments, an optional secondary agent can be the ingredient whose release is delayed. Suitable methods for delaying the release of an ingredient include, but are not limited to, coating or embedding the ingredients in material in polymers, wax, gels, and the like. Delayed release dosage formulations can be prepared as described in standard references such as “Pharmaceutical dosage form tablets,” eds. Liberman et. al. (New York, Marcel Dekker, Inc., 1989), “Remington—The science and practice of pharmacy”, 20^{th} ed., Lippincott Williams & Wilkins, Baltimore, MD, 2000, and “Pharmaceutical dosage forms and drug delivery systems”, 6^{th} Edition, Ansel et al., (Media, PA: Williams and Wilkins, 1995). These references provide information on excipients, materials, equipment, and processes for preparing tablets and capsules and delayed release dosage forms of tablets and pellets, capsules, and granules. The delayed release can be anywhere from about an hour to about 3 months or more.

Examples of suitable coating materials include, but are not limited to, cellulose polymers such as cellulose acetate phthalate, hydroxypropyl cellulose, hydroxypropyl methylcellulose, hydroxypropyl methylcellulose phthalate, and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Coatings may be formed with a different ratio of water-soluble polymer, water insoluble polymers, and/or pH dependent polymers, with or without water insoluble/water soluble non-polymeric excipient, to produce the desired release profile. The coating is either performed on the dosage form (matrix or simple) which includes, but is not limited to, tablets (compressed with or without coated beads), capsules (with or without coated beads), beads, particle compositions, “ingredient as is” formulated as, but not limited to, suspension form or as a sprinkle dosage form.

Where appropriate, the dosage forms described herein can be a liposome. In these embodiments, primary active ingredient(s), and/or optional secondary active ingredient(s), and/or pharmaceutically acceptable salt thereof where appropriate are incorporated into a liposome. In embodiments where the dosage form is a liposome, the pharmaceutical formulation is thus a liposomal formulation. The liposomal formulation can be administered to a subject in need thereof.

Dosage forms adapted for topical administration can be formulated as ointments, creams, suspensions, lotions, powders, solutions, pastes, gels, sprays, aerosols, or oils. In some embodiments for treatments of the eye or other external tissues, for example the mouth or the skin, the pharmaceutical formulations are applied as a topical ointment or cream. When formulated in an ointment, a primary active ingredient, optional secondary active ingredient, and/or pharmaceutically acceptable salt thereof where appropriate can be formulated with a paraffinic or water-miscible ointment base. In other embodiments, the primary and/or secondary active ingredient can be formulated in a cream with an oil-in-water cream base or a water-in-oil base. Dosage forms adapted for topical administration in the mouth include lozenges, pastilles, and mouth washes.

Dosage forms adapted for nasal or inhalation administration include aerosols, solutions, suspension drops, gels, or dry powders. In some embodiments, a primary active ingredient, optional secondary active ingredient, and/or pharmaceutically acceptable salt thereof where appropriate can be in a dosage form adapted for inhalation is in a particle-size-reduced form that is obtained or obtainable by micronization. In some embodiments, the particle size of the size reduced (e.g., micronized) compound or salt or solvate thereof, is defined by a D50 value of about 0.5 to about 10 microns as measured by an appropriate method known in the art. Dosage forms adapted for administration by inhalation also include particle dusts or mists. Suitable dosage forms wherein the carrier or excipient is a liquid for administration as a nasal spray or drops include aqueous or oil solutions/suspensions of an active (primary and/or secondary) ingredient, which may be generated by various types of metered dose pressurized aerosols, nebulizers, or insufflators. The nasal/inhalation formulations can be administered to a subject in need thereof.

In some embodiments, the dosage forms are aerosol formulations suitable for administration by inhalation. In some of these embodiments, the aerosol formulation contains a solution or fine suspension of a primary active ingredient, secondary active ingredient, and/or pharmaceutically acceptable salt thereof where appropriate and a pharmaceutically acceptable aqueous or non-aqueous solvent. Aerosol formulations can be presented in single or multi-dose quantities in sterile form in a sealed container. For some of these embodiments, the sealed container is a single dose or multi-dose nasal or an aerosol dispenser fitted with a metering valve (e.g., metered dose inhaler), which is intended for disposal once the contents of the container have been exhausted.

Where the aerosol dosage form is contained in an aerosol dispenser, the dispenser contains a suitable propellant under pressure, such as compressed air, carbon dioxide, or an organic propellant, including but not limited to a hydrofluorocarbon. The aerosol formulation dosage forms in other embodiments are contained in a pump-atomizer. The pressurized aerosol formulation can also contain a solution or a suspension of a primary active ingredient, optional secondary active ingredient, and/or pharmaceutically acceptable salt thereof. In further embodiments, the aerosol formulation also contains co-solvents and/or modifiers incorporated to improve, for example, the stability and/or taste and/or fine particle mass characteristics (amount and/or profile) of the formulation. Administration of the aerosol formulation can be once daily or several times daily, for example 2, 3, 4, or 8 times daily, in which 1, 2, 3 or more doses are delivered each time. The aerosol formulations can be administered to a subject in need thereof.

For some dosage forms suitable and/or adapted for inhaled administration, the pharmaceutical formulation is a dry powder inhalable-formulations. In addition to a primary active agent, optional secondary active ingredient, and/or pharmaceutically acceptable salt thereof where appropriate, such a dosage form can contain a powder base such as lactose, glucose, trehalose, mannitol, and/or starch. In some of these embodiments, a primary active agent, secondary active ingredient, and/or pharmaceutically acceptable salt thereof where appropriate is in a particle-size reduced form. In further embodiments, a performance modifier, such as L-leucine or another amino acid, cellobiose octaacetate, and/or metals salts of stearic acid, such as magnesium or calcium stearate. In some embodiments, the aerosol formulations are arranged so that each metered dose of aerosol contains a predetermined amount of an active ingredient, such as the one or more of the compositions, compounds, vector(s), molecules, cells, and combinations thereof described herein.

Dosage forms adapted for vaginal administration can be presented as pessaries, tampons, creams, gels, pastes, foams, or spray formulations. Dosage forms adapted for rectal administration include suppositories or enemas. The vaginal formulations can be administered to a subject in need thereof.

Dosage forms adapted for parenteral administration and/or adapted for injection can include aqueous and/or non-aqueous sterile injection solutions, which can contain antioxidants, buffers, bacteriostats, solutes that render the composition isotonic with the blood of the subject, and aqueous and non-aqueous sterile suspensions, which can include suspending agents and thickening agents. The dosage forms adapted for parenteral administration can be presented in a single-unit dose or multi-unit dose containers, including but not limited to sealed ampoules or vials. The doses can be lyophilized and re-suspended in a sterile carrier to reconstitute the dose prior to administration. Extemporaneous injection solutions and suspensions can be prepared in some embodiments, from sterile powders, granules, and tablets. The parenteral formulations can be administered to a subject in need thereof.

For some embodiments, the dosage form contains a predetermined amount of a primary active agent, secondary active ingredient, and/or pharmaceutically acceptable salt thereof where appropriate per unit dose. In an embodiment, the predetermined amount of primary active agent, secondary active ingredient, and/or pharmaceutically acceptable salt thereof where appropriate can be an effective amount, a least effect amount, and/or a therapeutically effective amount. In other embodiments, the predetermined amount of a primary active agent, secondary active agent, and/or pharmaceutically acceptable salt thereof where appropriate, can be an appropriate fraction of the effective amount of the active ingredient.

### Co-Therapies and Combination Therapies

In some embodiments, the pharmaceutical formulation(s) described herein are part of a combination treatment or combination therapy. The combination treatment can include the pharmaceutical formulation described herein and an additional treatment modality. The additional treatment modality can be a chemotherapeutic, a biological therapeutic, surgery, radiation, diet modulation, environmental modulation, a physical activity modulation, and combinations thereof.

In some embodiments, the co-therapy or combination therapy can additionally include but not limited to, polynucleotides, amino acids, peptides, polypeptides, antibodies, aptamers, ribozymes, hormones, immunomodulators, antipyretics, anxiolytics, antipsychotics, analgesics, antispasmodics, anti-inflammatoir anti-histamines, anti-infectives, chemotherapeutics, and combinations thereof.

### Administration of the Pharmaceutical Formulations

The pharmaceutical formulations or dosage forms thereof described herein can be administered one or more times hourly, daily, monthly, or yearly (e.g., 1, 2, 3, 4, 5, 6, 7, 8, 9, 10, 11, 12, 13, 14, 15, 16, 17, 18, 19, 20, or more times hourly, daily, monthly, or yearly). In some embodiments, the pharmaceutical formulations or dosage forms thereof described herein can be administered continuously over a period of time ranging from minutes to hours to days. Devices and dosages forms are known in the art and described herein that are effective to provide continuous administration of the pharmaceutical formulations described herein. In some embodiments, the first one or a few initial amount(s) administered can be a higher dose than subsequent doses. This is typically referred to in the art as a loading dose or doses and a maintenance dose, respectively. In some embodiments, the pharmaceutical formulations can be administered such that the doses over time are tapered (increased or decreased) overtime so as to wean a subject gradually off of a pharmaceutical formulation or gradually introduce a subject to the pharmaceutical formulation.

As previously discussed, the pharmaceutical formulation can contain a predetermined amount of a primary active agent, secondary active agent, and/or pharmaceutically acceptable salt thereof where appropriate. In some of these embodiments, the predetermined amount can be an appropriate fraction of the effective amount of the active ingredient. Such unit doses may therefore be administered once or more than once a day, month, or year (e.g., 1, 2, 3, 4, 5, 6, or more times per day, month, or year). Such pharmaceutical formulations may be prepared by any of the methods well known in the art.

Where co-therapies or multiple pharmaceutical formulations are to be delivered to a subject, the different therapies or formulations can be administered sequentially or simultaneously. Sequential administration is administration where an appreciable amount of time occurs between administrations, such as more than about 15, 20, 30, 45, 60 minutes or more. The time between administrations in sequential administration can be on the order of hours, days, months, or even years, depending on the active agent present in each administration. Simultaneous administration refers to administration of two or more formulations at the same time or substantially at the same time (e.g., within seconds or just a few minutes apart), where the intent is that the formulations be administered together at the same time.

### Kits

Any of the compounds, compositions, formulations, particles, cells, described herein or a combination thereof can be presented as a combination kit. As used herein, the terms “combination kit” or “kit of parts” refers to the compounds, compositions, formulations, particles, cells and any additional components that are used to package, sell, market, deliver, and/or administer the combination of elements or a single element, such as the active ingredient, contained therein. Such additional components include, but are not limited to, packaging, syringes, blister packages, bottles, and the like. When one or more of the compounds, compositions, formulations, particles, cells, described herein or a combination thereof (e.g., agents) contained in the kit are administered simultaneously, the combination kit can contain the active agents in a single formulation, such as a pharmaceutical formulation, (e.g., a tablet) or in separate formulations. When the compounds, compositions, formulations, particles, and cells described herein or a combination thereof and/or kit components are not administered simultaneously, the combination kit can contain each agent or other component in separate pharmaceutical formulations. The separate kit components can be contained in a single package or in separate packages within the kit.

In some embodiments, the combination kit also includes instructions printed on or otherwise contained in a tangible medium of expression. The instructions can provide information regarding the content of the compounds, compositions, formulations, particles, cells, described herein or a combination thereof contained therein, safety information regarding the content of the compounds, compositions, formulations (e.g., pharmaceutical formulations), particles, and cells described herein or a combination thereof contained therein, information regarding the dosages, indications for use, and/or recommended treatment regimen(s) for the compound(s) and/or pharmaceutical formulations contained therein. In some embodiments, the instructions can provide directions for administering the compounds, compositions, formulations, particles, and cells described herein or a combination thereof to a subject in need thereof. In some embodiments, the subject in need thereof can need a treatment or prevention for a cardiac arrythmia and/or other cardiac contractility/fibrillation disorder and/or dysfunction, an epilepsy, a neurodegenerative disease (including, but not limited to, Alzheimer's Disease, dementias, and/or the like), a neuropathy, pain, cancer (including but not limited to cervical cancer, breast cancer, and prostate cancer), an Autism spectrum disorder, a mood disorder, or any combination thereof.

### Methods of Using Scn1B Mimetic Peptides

In some embodiments, the SCN1B mimetic peptides inhibit or otherwise modulate the function and/or activity of a native or endogenous SCN1B polypeptides. In some embodiments, the SCN1B mimetic peptides inhibit or otherwise modulate the function and/or activity of a voltage gated sodium channel formed of at least one beta 1 subunit. In some embodiments, native or endogenous SCN1B polypeptide activity is reduced 1-1,000 or more fold by one or more of the SCN1B mimetic peptides. In some embodiments, activity and/or function of a voltage gated sodium channel that incorporates a beta 1 subunit is reduced 1-1,000 or more fold by one or more of the SCN1B mimetic peptides.

Described in several example embodiments are methods of using the SCN1B mimetic peptides, encoding polynucleotides, nucleic acid constructs, cells, and/or the like for treatment and/or prevention of a disease, disorder, condition or symptom thereof whose pathophysiology can be modulated by inhibiting the function and/or activity of the native SCN1B subunit via the SCN1B mimetic peptides. Exemplary diseases, disorders, conditions include, without limitation cardiac arrythmias and other cardiac contractility/fibrillation disorders and/or dysfunctions, epilepsies neurodegenerative diseases (including, but not limited to, Alzheimer's Disease, dementias, and/or the like), neuropathies and pain, cancer (including but not limited to cervical cancer, breast cancer, and prostate cancer), and others (e.g., Autism spectrum and other mood disorders).

In some embodiments, a method of treating a SCN1B disease, condition, or disorder in a subject in need thereof, the method including administering, to the subject in need thereof, one or more engineered SCN1B mimetic polypeptides described herein, one or more SCN1B mimetic polypeptide encoding polynucleotide described herein, a vector or vector system containing one or more SCN1B mimetic polypeptides encoding polynucleotide described herein, a delivery vehicle containing one or more engineered SCN1B mimetic polypeptides described herein, one or more SCN1B mimetic polypeptide encoding polynucleotide described herein, a vector or vector system containing one or more SCN1B mimetic polypeptides encoding polynucleotide described herein, a cell or cell population containing or capable of expressing one or more engineered SCN1B mimetic polypeptides described herein, one or more SCN1B mimetic polypeptide encoding polynucleotide described herein, a vector or vector system containing one or more SCN1B mimetic polypeptides encoding polynucleotide described herein, a pharmaceutical formulation thereof, or any combination thereof. Administration can be by any suitable route. Exemplary administration routes are described elsewhere herein.

In some embodiments, administration is acute (i.e., administration occurs one or more times over a time period of 0 (single dose) to 2 hours or occurs such that activity of the SCN1B mimetic polypeptide(s) occurs for up to 2 hours). In some embodiments, administration is long-term (i.e., administration occurs one or more times over a period of time more than 2 hours, (e.g., 2, 4, 6, 8, 12, 24, 48, or more hours) or occurs such that the activity of the SCN1B mimetic polypeptide(s) occurs for more than 2 hours (e.g., 2, 4, 6, 8, 12, 24, 48, or more hours). In some embodiments, acute administration and/or activity of the SCN1B mimetic polypeptide(s) can have different therapeutic effects than long-term administration and/or activity of the SCN1B mimetic polypeptide(s).

In some embodiments, the subject in need thereof has or is suspected of having a cardiac arrythmia and/or other cardiac contractility/fibrillation disorder and/or dysfunction, an epilepsy, a neurodegenerative disease (including, but not limited to, Alzheimer's Disease, dementias, and/or the like), a neuropathy, pain, cancer (including but not limited to cervical cancer, breast cancer, and prostate cancer), an Autism spectrum disorder, a mood disorder, or any combination thereof.

Further embodiments are illustrated in the following Examples which are given for illustrative purposes only and are not intended to limit the scope of the invention.

### EXAMPLES

Now having described the embodiments of the present disclosure, in general, the following Examples describe some additional embodiments of the present disclosure. While embodiments of the present disclosure are described in connection with the following examples and the corresponding text and figures, there is no intent to limit embodiments of the present disclosure to this description. On the contrary, the intent is to cover all alternatives, modifications, and equivalents included within the spirit and scope of embodiments of the present disclosure. The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

### Example 1

The number of sudden cardiac deaths in the US is between 180,000 and 450,000 each year. (1, 2) Cardiac arrhythmias contribute to a high number of those. (4) Many drugs created to prevent arrhythmogenesis target ion channels in the heart, but there are often serious side effects. (5) For instance, the Cardiac Arrhythmia Suppression Trial (CAST), was successful in suppressing ventricular arrhythmias, but total deaths caused by arrhythmia increased during the trial. (6, 7) Thus, a critical barrier to progress in the field is design and use of anti-arrhythmic drugs that can successfully prevent arrhythmias, while not compromising overall cardiovascular health._The SCN1B-targeting peptides can improve upon current technology in preventing arrhythmias, and we contemplate doing so without the serious side effects. Without being bound by theory, this Example demonstrates that by mimicking adhesion function and targeting the natural breakdown of the voltage gated sodium channel (VGSC) β1 subunit using SCN1B mimetic peptides, it is possible to modulate β1 function in a manner that is beneficial to the treatment of cardiac arrhythmias and other diseases where β1 function modulation may be warranted. Moreover, the compositions of the present disclosure are contemplated to provide benefit in other diseases including those in which VGSCs are implicated. These include neurodegenerative diseases such as Alzheimer's disease, epilepsy, and cancer.

The perinexus is a specialized nano-domain of the cardiac intercalated disc directly adjacent to gap junctions. (8) The perinexus is within 30 nm in width under normal conditions and it has been shown that widening of the perinexus leads to arrythmogenesis, suggesting that the regulation of perinexal width is a target for treating arrhythmias. (3, 9) The subunits of the voltage gated VGSC complex, Nax1.5 and SCN1B (β1), localize in the perinexus. (3, 10) β1 has a variety of roles in regulating channel properties and as an adhesion molecule. (11) β1 is also implicated in a variety of cardiac pathologies, including Brugada syndrome and atrial fibrillation. (12, 13) Knock down of β1 in mouse heart results in widened perinexus and increased arrhythmogenesis. (3)

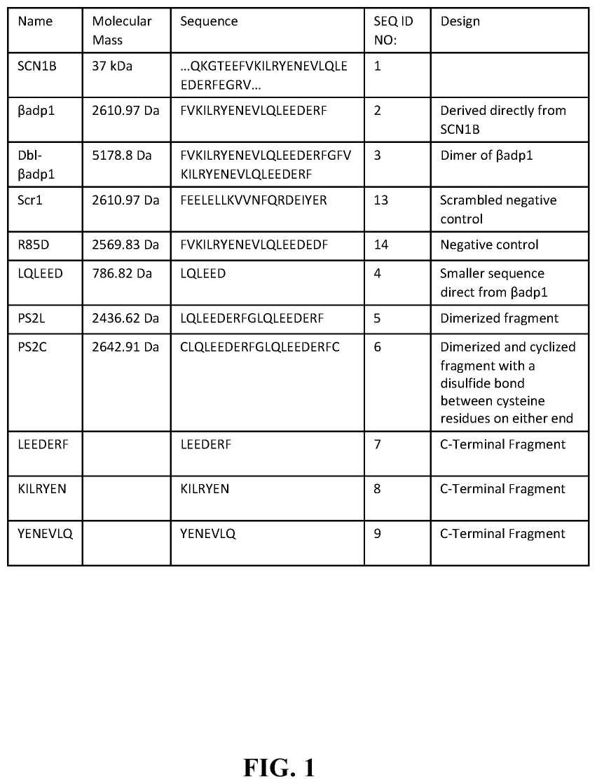

To target the β1 subunit, a 19 amino acid (AA) peptide was developed and named βadp1 that mimics the extracellular Ig loop domain of β1, which is responsible for its adhesion function. Isolated guinea pig hearts treated acutely with βadp1 showed decreased conduction velocity and a widened perinexus (3) To understand more about the mechanism by which βadp1 causes these physiological changes, we monitored cells treated with βadp1 over prolonged time periods up to 48 hours from the initial treatment. This resulted in the unexpected observation that at 48 hours β1 levels were notably increased. Earlier work carried out and reported by the laboratory had focused on earlier time-points (<2 hours), wherein βadp1 was found to cause perinexal de-adhesion. (3) The fragments of βadp1 were further tested, unexpectedly identifying utility of optimized smaller sequences of 6-9 AAs and a dimerized version of βadp1 called Db1-βadp1 was generated in addition to dimers of these short sequences called βS2L and βS2C. Short sequences from the N-terminus through to the C-terminus based on βadp1 were tested and it was unexpectedly found that only C-terminal peptides showed useful levels of activity. This identified previously unknown and unexpected peptide sequences based on SCN1B that can have therapeutic benefit in treating arrhythmias, as well as other SCN1B mediated diseases, conditions, and disorders. These exemplary sequences are shown in FIG. 1 in comparison to a portion of SCN1B (amino acids 61-90) with the single letter abbreviations commonly used for amino acids, as well as figures and tables of this disclosure.

Recent research has highlighted the regulated intramembrane proteolysis (RIP) of β1, in which β1 is sequentially cleaved by BACE1 and γ-secretase and the intracellular domain translocates to the nucleus altering transcription of multiple genes, including the VGSC complex as shown in FIG. 2. (14-16) This data indicates that βadp1 prompts a change in the abundance of β1 and the c-terminal fragment (CTF) after treatment, indicating that βadp1 acts on this pathway, which is a novel use for a peptide and a new mechanism by which βadp1 can mediate effects on cells, tissues and the heart as a whole. Interestingly, there is an endogenously expressed secreted isoform of β1 called β1b comprising solely the extracellular Ig domain. Loss-of-function mutations in β1b have been associated with increased susceptibility to atrial arrhythmias, Long Q T and Brugada syndromes, and epilepsy. (17, 18) βadp1 was rationally designed to mimic and adhere to Ig domain sequence found in both βl and β1b and may feasibly act as a novel ligand that recapitulates aspects of β1b in maintaining normal heart and brain function.

### Results

The short term effects of βadp1 on Chinese hamster lung 1610 cells that stably express SCN1B (β1) (1610ß1) was previously observed. (3) To determine the long-term effects of treatment, cells were treated for 6, 24, and 48 hours, the abundance and functionality of β1 at those timepoints were analyzed.

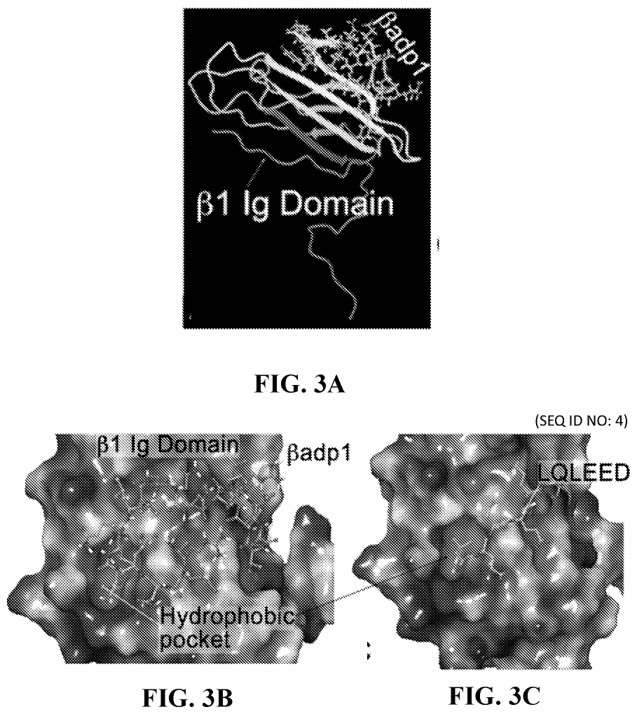

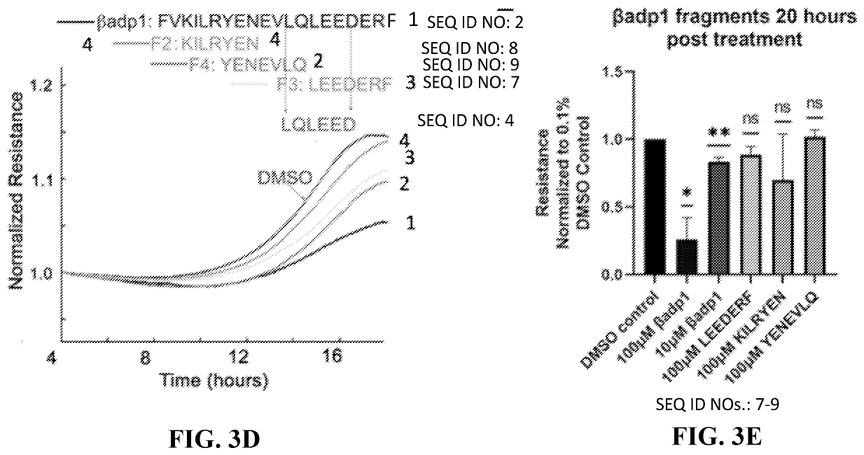

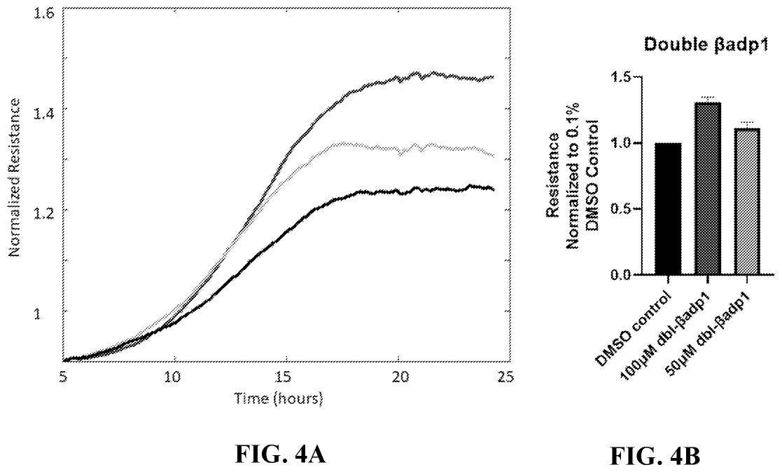

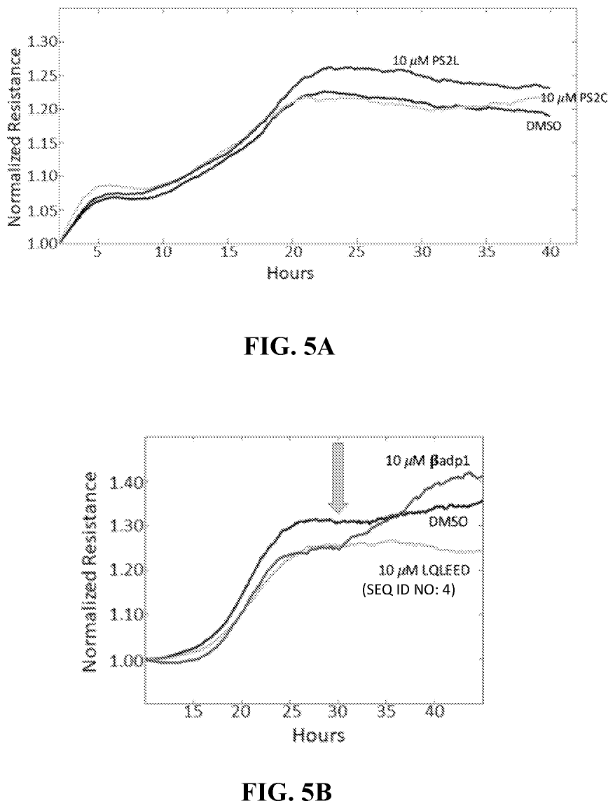

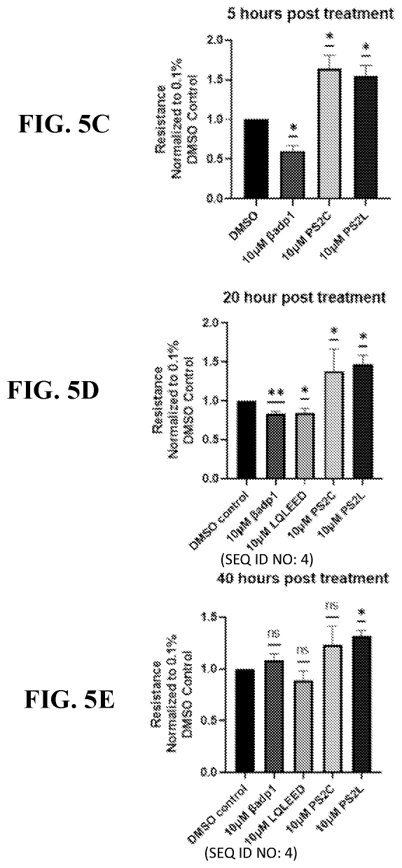

In this Example to evaluate the long term effects (effects after more than 2 hours post treatment) of βadp1 and derivatives on β1 cell adhesion, electric cell-substrate impedance sensing (ECIS) was employed. ECIS measures the impedance of electric current. 4000 Hz is the recommended frequency for measuring changes in impedance between cells, assaying intercellular adhesion levels, as mediated by β1. Resistance is calculated from impedance and used as a measure of β1 activity, with an increased resistance indicating increased β1 adhesion. To assist in focusing on the active sequence of βadp1 used to create the other peptides in this disclosure, ECIS experiments were completed using multiple small sequences as shown in FIGS. 3A-3E. From this, and in combination with the in silico experiments, it was determined that LQLEED (SEQ ID NO: 4) was the optimal sequence. It was also examined if Db1-βadp1 had similar or enhanced effects, and ECIS experiments unexpectedly showed that Db1-βadp1 increases resistance over 24 hours after treatment in 1610 cells, opposite to effects of monomer βadp1 (FIGS. 4A-4B). For the other dimerized peptides, ERF residues were included on the c-terminal end of the LQLEED (SEQ ID NO: 4) peptide because residue R85 in SCN1B is associated with atrial fibrillation. Cells were plated in 8 well plates lined with 40 electrodes and treated with a 10 μM concentration of βadp1 in a final concentration of 0.1% DMSO at the time of plating or DMSO vehicle. There are cell-free wells in each experiment to account for any potential impact the cell culture media has on impedance. βadp1 significantly decreased resistance compared to the DMSO control up to 20 hours, consistent with previous results (3) (FIGS. 5A-5E). However, by 40 hours the effects of βadp1 had reversed. The graph in FIG. 5B shows a clear change and inflection occurring around 30 hours after treatment as indicated by a green arrow. LQLEED (SEQ ID NO: 4) showed a similar profile, with decreased resistance up to 20 hours, and by 40 hours the effects of LQLEED (SEQ ID NO: 4) had reversed and resulted in promoting adhesion. Importantly and in contrast, the dimerized peptides βS2C and βS2L both increased resistance throughout the time course (excluding 48 hours with βS2C), showing facilitation of β1 adhesion by the dimers, with no acute de-adhesion effects detectable.

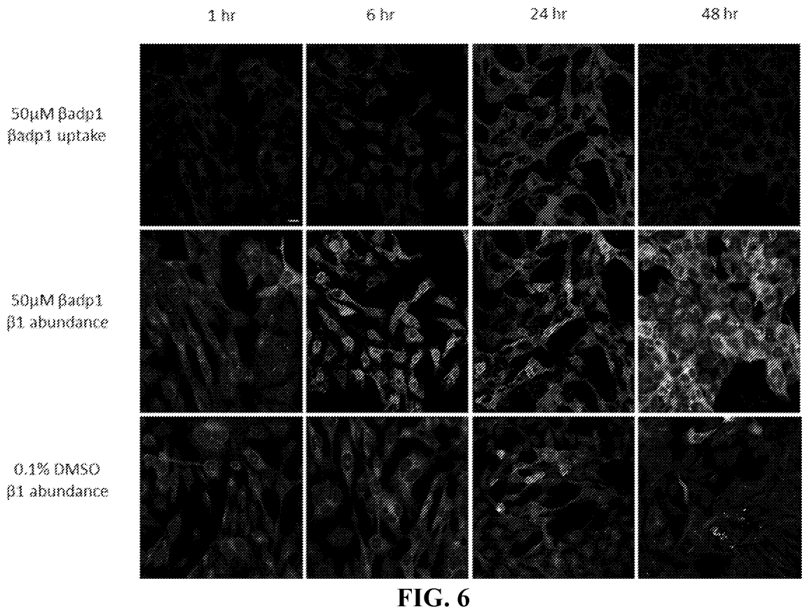

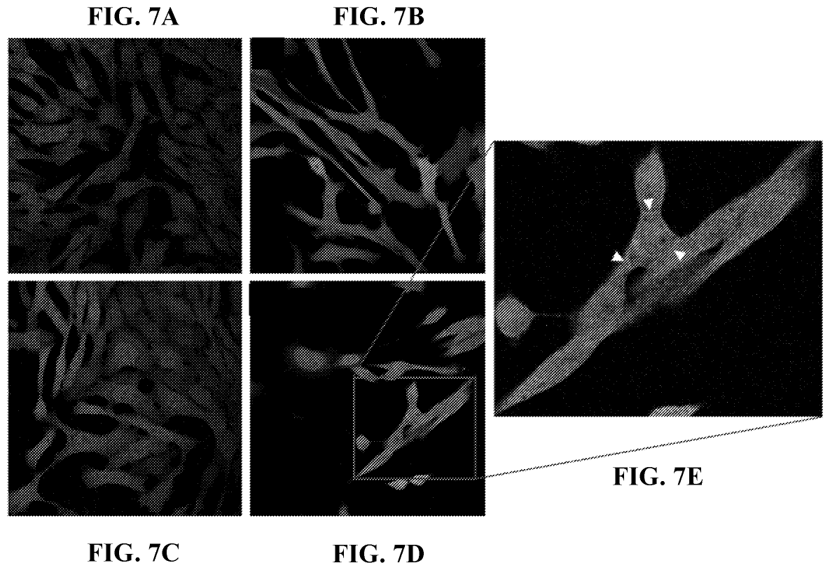

Next, the abundance of β1 protein was evaluated using confocal microscopy immunofluorescence (IF). 1610β1 cells were plated in 12 well plates on lysine coated coverslips and allowed to grow for 48 hours. Cells were then treated with a 50 μM concentration of βadp1, βS2L, βS2C, or LQLEED (SEQ ID NO: 4) or βadp1 conjugated with a Biotin molecule (B-βadp1) solubilized in a final concentration of 0.1% dimethyl sulfoxide (DMSO). Controls for the experiment were cells treated with only 0.1% DMSO vehicle. Cells were then fixed in 4% paraformaldehyde and stained with an antibody against the extracellular domain of β1 (Virginia Tech) and a streptavidin-Alexa647 (Invitrogen) conjugate to stain B-βadp1. FIG. 6 shows the greatest uptake of B-βadp1 occurs at 24 hours and there is an increase in β1 abundance following this uptake 24 hours later. FIGS. 7A-7E shows that βS2C, βS2L, and to a lesser extent LQLEED (SEQ ID NO: 4) also all show upregulation of β1 protein 48 hours after treatment. In addition, βS2L especially appears to increase the β1 present in the membrane and particularly in membranes of cells that are directly adjacent (white arrows). The DMSO-treated cells show little change in β1 levels following treatment.

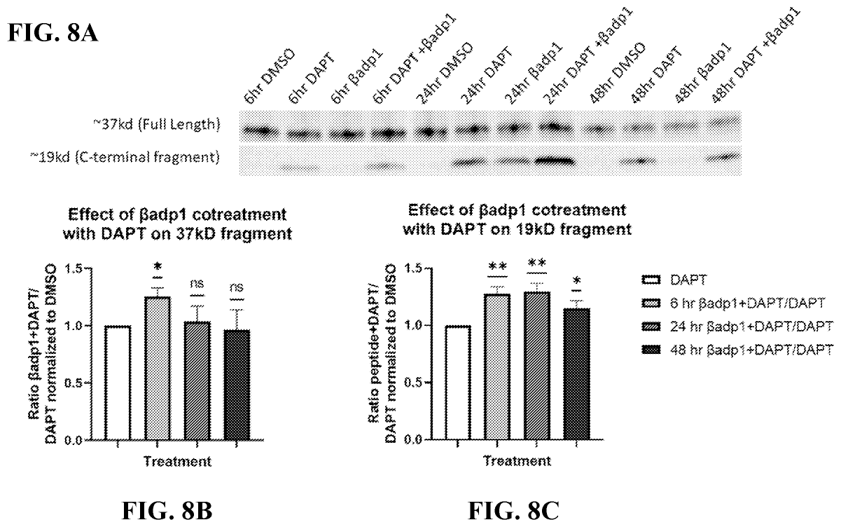

β1 abundance was further examined using Western blotting (WB) at time points of 6, 24, and 48 hours after initial βadp1 treatment. Cells were plated in 35 mm cell culture dishes and treated with 50 μM βadp1 or βS2L solubilized in a final concentration of 0.1% DMSO at the time of plating. The control was DMSO vehicle. To determine whether βadp1 affects the RIP of β1, cells were also treated with 1 μM DAPT, an inhibitor of γ-secretase, to cause an accumulation of the intermediate substrate in the RIP of β1, the CTF. Whole cell lysate was collected and run on stain free gels and transferred to a Polyvinylidene difluoride (PVDF) membrane. Membranes were labeled with an antibody against the c-terminal domain of β1 (Cell Signaling Technologies). FIGS. 8A-8C show that DAPT results in an accumulation of the 19KD CTF as shown previously by the Isom group (14). Furthermore, cotreatment with βadp1 and DAPT results in a greater accumulation of the CTF at every timepoint tested, 6, 24, and 48 hours, suggesting that βadp1 increases the RIP of β1.

The data shows that βadp1 sets in motion a cascade of events that leads to modulation of 1 expression and therefore effects on adhesion during various periods of treatment. Without being bound by theory, understanding that β1 undergoes RIP (14), and based on our data that βadp1 causes an increase in the intermediate fragment (CTF) of RIP, we contemplate that βadp1 and the optimized short and dimerized sequences can modulate RIP, resulting in effects on gene transcription and protein trafficking, including VGSC ion channels to the membrane. (FIG. 2). It is envisioned that other short sequences based on the CT of βadp1, with conservative substitutions of naturally occurring or non-naturally occurring amino acids (e.g., D and L isomers of AAs) can have the same beneficial effects, whether in single or dimerized forms.

Based on this information and without being bound by theory that βadp1 and its derivatives can unexpectedly be used as a new class of therapeutics for diseases, conditions, and disorders, where the SCN1B channel is implicated. For example, the βadp1 and its derivatives can unexpectedly be used as an anti-arrhythmic drug. It has been shown previously that disruption of and knockdown of β1 in mouse hearts leads to arrhythmogenesis. Thus, by increasing the level of β1 present in the cells, we can beneficially modulate β1 adhesion levels providing a means of treating arrhythmia.

Another exemplary use for βadp1, its derivatives, and formulations thereof can include as treatments for Alzheimer's disease. The proteolysis of amyloid precursor protein (APP) causes the creation and buildup of amyloid-β plaques, which are a hallmark of Alzheimer's disease. The proteolysis of APP follows the same sequential cleavage steps that β1 undergoes, suggesting potential for our Scn1b targeting peptide ligands to impact this cleavage process in a similar manner to that of β1 proteolysis.

The peptides of the present disclosure can also be indicated for treating generalized epilepsy with febrile seizures, as SCN1B mutations are found in patients with this disease (19) and for treating breast cancer, as it has been shown that β1 can promote tumor growth and metastasis in breast and other cancers the β subunit inhibitory peptides of the present disclosure can thus also be indicated for treating cancer. (20)

Scn1b is one of a family of 3 known VGSC beta subunits (i.e., in addition to β1b—these include Scn2b, Scn3b, and Scn4b) and has homologies to other Ig-containing cell adhesion proteins such as P0. It is envisioned that short Ig-based peptides based on adhesion domains within the Ig loop homologous to the βadp1 sequence (as well as dimers of these sequences) can have similar effects on RIP as the Scn1b mimetic peptides we describe herein, and related signaling processes, effecting gene transcription.

### References for Example 1

1. Deo R, Albert C M. Epidemiology and genetics of sudden cardiac death. Circulation. 2012; 125 (4): 620-37. Epub 2012 Feb. 2. doi: 10.1161/circulationaha.111.023838. PubMed PMID: 22294707; PMCID: PMC3399522.
2. Khurshid S, Choi S H, Weng L-C, Wang E Y, Trinquart L, Benjamin E J, Ellinor P T, Lubitz S A. Frequency of Cardiac Rhythm Abnormalities in a Half Million Adults. Circulation: Arrhythmia and Electrophysiology. 2018; 11 (7): e006273. doi doi: 10.1161/CIRCEP.118.006273.
3. Veeraraghavan R, Hoeker G S, Alvarez-Laviada A, Hoagland D, Wan X, King D R, Sanchez-Alonso J, Chen C, Jourdan J, Isom L L, Deschenes I, Smyth J W, Gorelik J, Poelzing S, Gourdie R G. The adhesion function of the sodium channel beta subunit (β1) contributes to cardiac action potential propagation. Elife. 2018; 7: e37610. doi: 10.7554/eLife.37610. PubMed PMID: 30106376.
4. Garcia-Elias A, Benito B. Ion Channel Disorders and Sudden Cardiac Death. Int J Mol Sci. 2018; 19 (3): 692. PubMed PMID: doi: 10.3390/ijms19030692.
5. Vidhya Rao M P N. Current and Potential Antiarrhythmic Drugs Targeting Voltage-Gated Cardiac Ion Channels. Cardiovascular Pharmacology: Open Access. 2015;04 (02). doi: 10.4172/2329-6607.1000139.
6. Echt D S, Liebson P R, Mitchell L B, Peters R W, Obias-Manno D, Barker A H, Arensberg D, Baker A, Friedman L, Greene H L, Huther M L, Richardson D W. Mortality and Morbidity in Patients Receiving Encainide, Flecainide, or Placebo. New England Journal of Medicine. 1991;324 (12): 781-8. doi: 10.1056/nejm199103213241201. PubMed PMID: 1900101.
7. Effect of the Antiarrhythmic Agent Moricizine on Survival after Myocardial Infarction. New England Journal of Medicine. 1992;327 (4): 227-33. doi: 10.1056/nejm 199207233270403. PubMed PMID: 1377359.
8. Rhett J M, Gourdie R G. The perinexus: a new feature of Cx43 gap junction organization. Heart Rhythm. 2012; 9 (4): 619-23. Epub 2011 Oct. 4. doi: 10.1016/j.hrthm.2011.10.003. PubMed PMID: 21978964.
9. Raisch T B, Yanoff M S, Larsen T R, Farooqui M A, King D R, Veeraraghavan R, Gourdie R G, Baker J W, Arnold W S, AlMahameed S T, Poelzing S. Intercalated Disk Extracellular Nanodomain Expansion in Patients With Atrial Fibrillation. Front Physiol. 2018; 9:398. Epub 2018 May 22. doi: 10.3389/fphys.2018.00398. PubMed PMID: 29780324; PMCID: PMC5945828.
10. Rhett J M, Ongstad E L, Jourdan J, Gourdie R G. Cx43 associates with Na (v) 1.5 in the cardiomyocyte perinexus. J Membr Biol. 2012; 245 (7): 411-22. Epub 2012 Jul. 19. doi: 10.1007/s00232-012-9465-z. PubMed PMID: 22811280.
11. Brackenbury W J, Isom L L. Voltage-gated Na+ channels: potential for beta subunits as therapeutic targets. Expert Opin Ther Targets. 2008; 12 (9): 1191-203. doi: 10.1517/14728222.12.9.1191. PubMed PMID: 18694383.
12. Watanabe H, Darbar D, Kaiser D W, Jiramongkolchai K, Chopra S, Donahue B S, Kannankeril P J, Roden D M. Mutations in sodium channel β1- and β2-subunits associated with atrial fibrillation. Circ Arrhythm Electrophysiol. 2009; 2 (3): 268-75. Epub 2009 Oct. 8. doi: 10.1161/circep. 108.779181. PubMed PMID: 19808477; PMCID: PMC2727725.
13. Watanabe H, Koopmann T T, Le Scouarnec S, Yang T, Ingram C R, Schott J J, Demolombe S, Probst V, Anselme F, Escande D, Wiesfeld A C, Pfeufer A, Kääb S, Wichmann H E, Hasdemir C, Aizawa Y, Wilde A A, Roden D M, Bezzina C R. Sodium channel β1 subunit mutations associated with Brugada syndrome and cardiac conduction disease in humans. J Clin Invest. 2008;118 (6): 2260-8. Epub 2008 May 10. doi: 10.1172/jci33891. PubMed PMID: 18464934; PMCID: PMC2373423.
14. Bouza A A, Edokobi N, Hodges S L, Pinsky A M, Offord J, Piao L, Zhao Y-T, Lopatin A N, Lopez-Santiago L F, Isom L L. Sodium channel β1 subunits participate in regulated intramembrane proteolysis-excitation coupling. JCI Insight. 2021; 6 (3). doi: 10.1172/jci.insight.141776.
15. Bouza A A, Philippe J M, Edokobi N, Pinsky A M, Offord J, Calhoun J D, Lopez-Florán M, Lopez-Santiago L F, Jenkins P M, Isom L L. Sodium channel β1 subunits are post-translationally modified by tyrosine phosphorylation, S-palmitoylation, and regulated intramembrane proteolysis. J Biol Chem. 2020; 295 (30): 10380-93. Epub 2020 Jun. 7. doi: 10.1074/jbc.RA120.013978. PubMed PMID: 32503841; PMCID: PMC7383382.
16. Wong H K, Sakurai T, Oyama F, Kaneko K, Wada K, Miyazaki H, Kurosawa M, De Strooper B, Saftig P, Nukina N. beta Subunits of voltage-gated sodium channels are novel substrates of beta-site amyloid precursor protein-cleaving enzyme (BACE1) and gamma-secretase. J Biol Chem. 2005;280 (24): 23009-17. Epub 2005 Apr. 13. doi: 10.1074/jbc.M414648200. PubMed PMID: 15824102.
17. Riuró H, Campuzano O, Arbelo E, Iglesias A, Batlle M, Pérez-Villa F, Brugada J, Pérez GJ, Scornik F S, Brugada R. A missense mutation in the sodium channel β1b subunit reveals SCN1B as a susceptibility gene underlying long QT syndrome. Heart Rhythm. 2014; 11 (7): 1202-9. Epub 2014 Mar. 26. doi: 10.1016/j.hrthm.2014.03.044. PubMed PMID: 24662403.
18. Edokobi N, Isom L L. Voltage-Gated Sodium Channel β1/β1B Subunits Regulate Cardiac Physiology and Pathophysiology. Front Physiol. 2018; 9:351. Epub 2018 May 10. doi: 10.3389/fphys.2018.00351. PubMed PMID: 29740331; PMCID: PMC5924814.
19. Scheffer I E, Harkin L A, Grinton B E, Dibbens L M, Turner S J, Zielinski M A, Xu R, Jackson G, Adams J, Connellan M, Petrou S, Wellard R M, Briellmann R S, Wallace R H, Mulley J C, Berkovic S F. Temporal lobe epilepsy and GEFS+ phenotypes associated with SCN1B mutations. Brain. 2007; 130 (Pt 1): 100-9. Epub 2006 Oct. 6. doi: 10.1093/brain/aw1272. PubMed PMID: 17020904.
20. Nelson M, Millican-Slater R, Forrest L C, Brackenbury W J. The sodium channel β1 subunit mediates outgrowth of neurite-like processes on breast cancer cells and promotes tumour growth and metastasis. Int J Cancer. 2014; 135 (10): 2338-51. Epub 2014 Apr. 26. doi: 10.1002/ijc.28890. PubMed PMID: 24729314.

### Example 2

βadp1 is a peptide mimetic of a 20 amino acid (aa) sequence from the immunoglobulin (Ig) domain of the voltage gated sodium channel (VGSC) β1 subunit involved in adhesive interactions [1]. Previously, Applicant has reported that βadp1 inhibits β1-mediated adhesion and gap junction-associated VGSC activity in cultured cells over time frames of 1 to 2 hours. Modeling in silico indicates that βadp1 likely mediates these effects by directly and selectively binding to the β1 extracellular Ig domain thereby inhibiting trans-adherent interactions between β1 molecules on neighboring cells (FIG. 3A-3B). Applicant synthesized a dimerized variant of the 20 aa βadp1 (FIG. 1) and tested it in monolayers of 1610 Chinese Hamster Lung cells stably expressing the β1 subunit (1610ß1) using electric cell substrate impedance sensing (ECIS) to determine whether it acted as an agonist that promoted β1-mediated adhesion. Consistent with previous results, 10 and 100 μM treatments with βadp1 monomer prompted significant decreases in ECIS-assayed adhesion in 1610β1 cells [1]. However, dimeric βadp1 (named Double-βadp1/Db1-βadp1) increased resistance compared to vehicle control (data not shown). This elevation was higher for 100 μM Db1-βadp1 versus the 50 μM treatment and was sustained over a 24 hour treatment period ECIS testing of Db1-βadp1 in a parental cell line of 1610 cells expressing negligible levels of β1 did not result in increased intercellular resistance.

### Rationale for Synthesis of Shorter Scn1b/β1 Ig Mimetic Inhibitory and Agonist Peptides

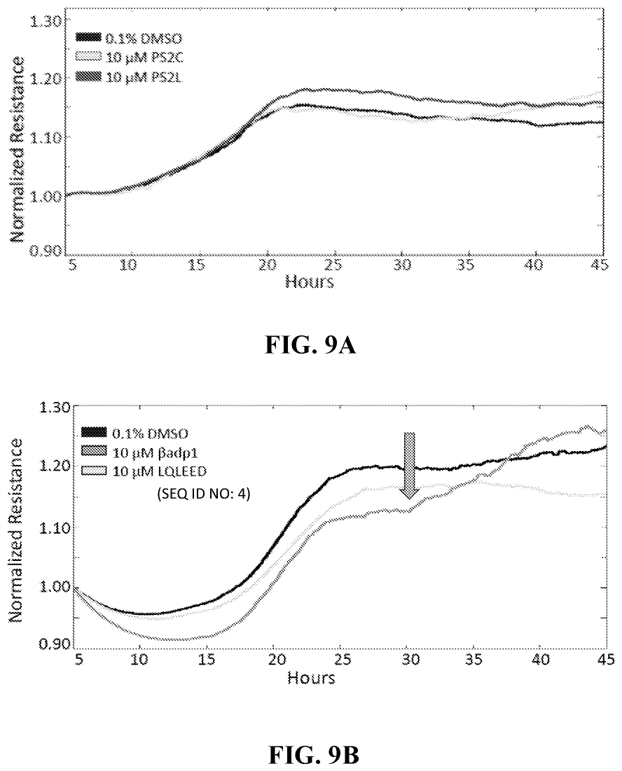

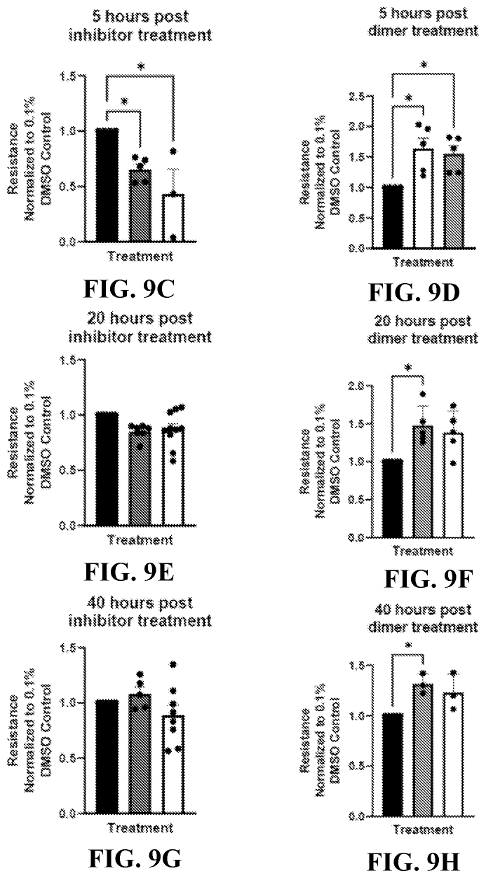

Db1-βadp is a 41 amino acid sequence comprising a flexible glycine linker between two βadp1 sequences. This dimer has a relatively large molecular mass for a peptide, exceeding 5000 daltons (FIG. 1). In the mammalian ventricle β1 is preferentially localized in intercalated discs between myocytes, which form a specialized compartment of extracellular space into which diffusion of molecules of 5 kD or more is likely restricted [1]. Thus, Db1-βadp1 is unlikely to readily diffuse into the tortuous and narrow confines of ID extracellular space. As a strategy to rationally design peptides that can efficiently target ID-localized β1 we used ECIS and 1610β1 cells to screen peptides based on the βadp1 sequence of less than 5 kD (see FIG. 1). It was found that 10 μM treatment with a short monomeric peptide, LQLEED (SEQ ID NO: 4), towards the carboxyl terminus (CT) of βadp1 decreased levels of intercellular resistance in 1610β1 cells in a manner similar to that observed for the larger 20aa peptide over an acute 5 hour time point (FIG. 9B). Whereas, more amino-terminally located peptides did not have inhibitory activity (data not shown). Modeling in silico suggested that LQLEED (SEQ ID NO: 4) organizes into a low energy pose with the Scn1b/β1 with its NT leucine residues embedded in a hydrophobic pocket on the putative adhesion surface of the Ig domain (FIG. 3B).

To investigate whether dimerization of shorter CT inhibitory monomers could generate agonists that functioned in a manner like Db1-βadp1, we synthesized LQLEEDERF-G-LQLEEDERF (SEQ ID NO: 5). This dimeric peptide, which is called βS2L, is a sequential repeat of the CT-most 9 amino acids of βadp1 including the LQLEED (SEQ ID NO: 4) sequence spaced by a glycine (G) linker. Applicant also synthesized a dimeric peptide of identical sequence, except that it included cysteine residues at its NT and CT. This novel peptide called βS2C (CLQLEEDERF-G-LQLEEDERFC (SEQ ID NO: 6)) was designed to be cyclizable via disulfide bonding between its cysteines. In ECIS at the acute 5 hours timepoint, resistance levels induced in 1610β1 cells by both the βS2L and βS2C dimers were significantly elevated over vehicle control (FIG. 9A). Notably, βS2L increased resistance to levels that exceeded those resulting from treatment by the LQLEED (SEQ ID NO: 4) inhibitory monomer by 5-6 fold.

### Differential Effects of βAdp1 and Derivatives on Cell Adhesion and β1 Immunolabeling Over 48 Hour Time Courses

With the effects of acute treatment with βadp1 and its derivatives established over time courses of 5 hours or less, we next sought to examine peptide effects on cellular adhesion over longer periods. 1610β1 cells were treated with 10 μM βadp1 and LQLEED (SEQ ID NO: 4) monomers and subjected to ECIS for 40 hours or more (FIG. 9B). Up to 20-hours, the effects of monomers cell adhesion were similar to that seen at acute timepoints previously [1], with significant decreases in resistance, compared to vehicle control (FIG. 9B). However, after 24-30 hours of treatment we noted a shift in the response, where resistance began to rise—as indicated by the arrow in FIG. 9B. In some ECIS runs, the rise in resistance in monomer-treated cells eventually exceeded that of control cells—as shown for βadp1 in FIG. 9B. Whilst this increase did not always move above control levels, it was sufficient on average that by 40 hours post-treatment effects of βadp1 and LQLEED (SEQ ID NO: 4) on cellular resistance were no longer significantly below those of controls, as had been the case at the earlier time points. Applicant performed similar long-term ECIS assays on the βS2L and βS2C dimeric peptides. βS2L performed as expected, significantly increasing resistance compared to vehicle control over the entire 40 hour time course of the experiment (FIG. 9A). Interestingly, βS2C also increased resistance at the 20-hour timepoint, but not at 40 hours post treatment (FIG. 9A), suggesting that βS2L may induce a consistent effect in 1610β1 cells over prolonged exposures. FIG. 9C-9H show the effect of treatment with the inhibitor (FIG. 9C, 9E, 9G) as compared to the dimer (FIG. 9D, 9F, 9H) at 5, 20, and 40 hours post treatment with the inhibitor or dimer.

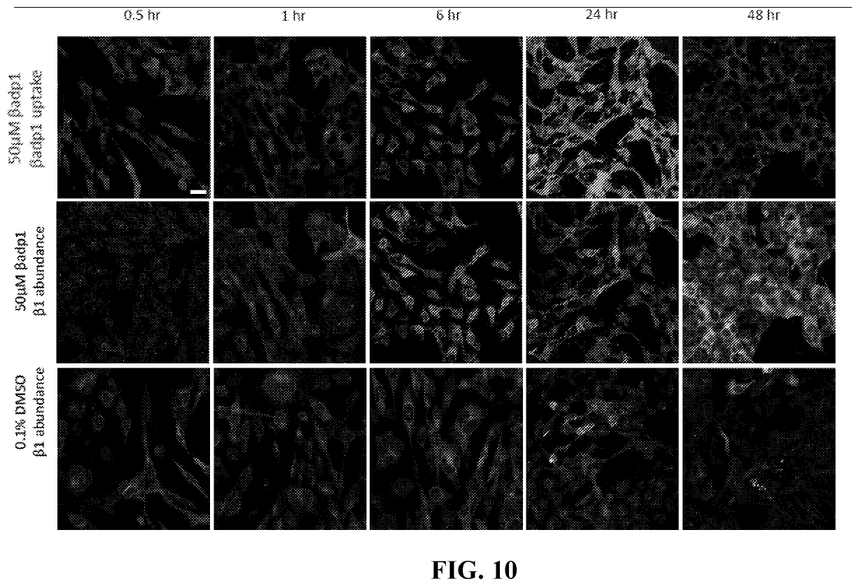

Applicant undertook immunolabeling studies to further probe the effect of the mimetic peptides on 1610β1 cells over time courses of up to 2 days. 1610β1 cells incubated with 50 μM Biotin-βadp1 showed accumulation of the peptide, which peaked at around 24 hours, declining at 48 hours (FIG. 10, top row). Interestingly, immunolabeling for the β1 protein itself indicated a steady increase in abundance in 1610β1 cells in response to βadp1, which reached maximal levels at 48 hours (FIG. 10, middle row), relative to control cells (FIG. 10, bottom row). Prompted by this observation we examined what effects of 10 μM concentrations of LQLEED (SEQ ID NO: 4) and βS2L on β1 immunolabeling at or 48 hours over the same time course. As was the case for βadp1, in response to LQLEED (SEQ ID NO: 4) β1 immunolabeling in 1610β1 cells appeared to increase over the 48 hour period (FIG. 7C). High magnification images indicated that this increased immunolabeling was cell-wide, including within the cytoplasm, with occasional evidence of increased intensity of immunolabeling signal at cell borders. βS2L treatment also led to cell-wide increases in β1 immunolabeling at 48-hours. However, importantly we noted that βS2L treatment prompted a distinctive increase in signal at cell borders, relative to controls and other peptides (FIG. 7D-7E).

### βAdp1 and βS2L Increase Cleavage of β1 Via Regulated Intramembrane Proteolysis

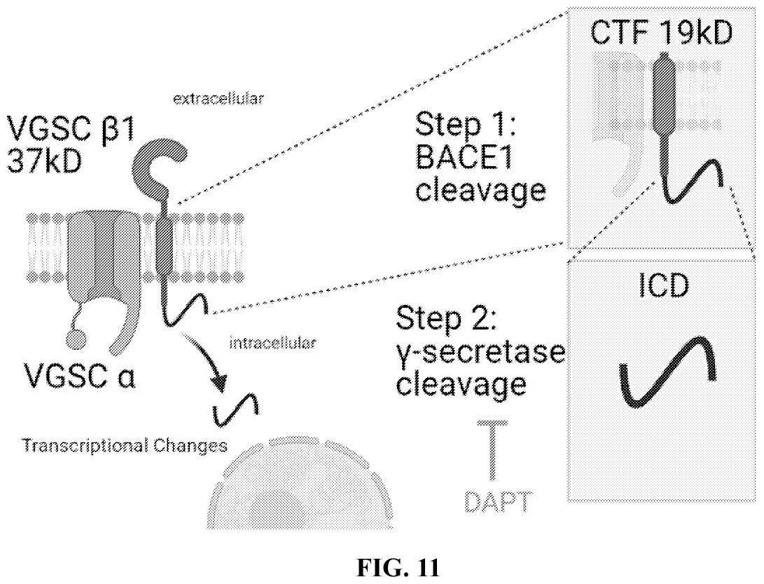

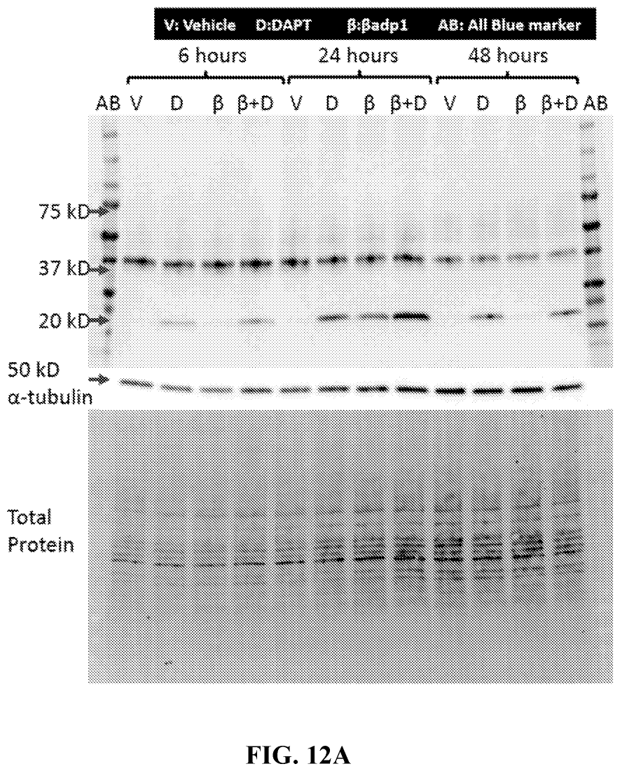

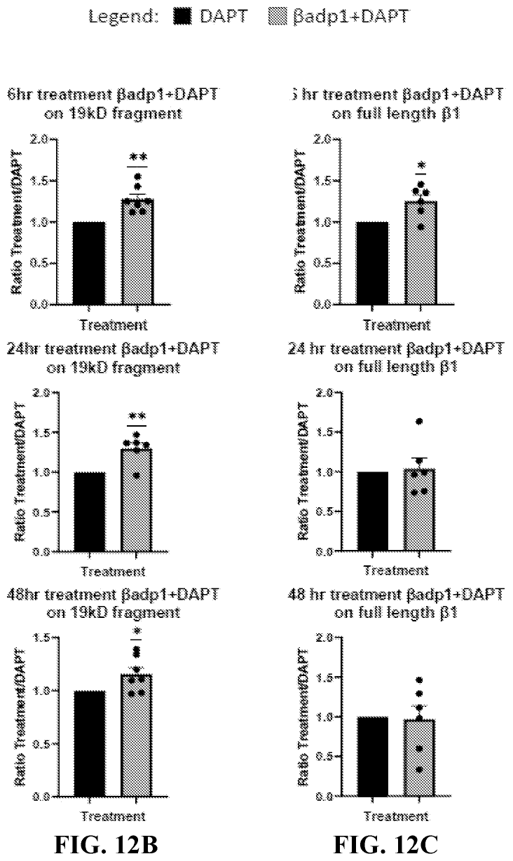

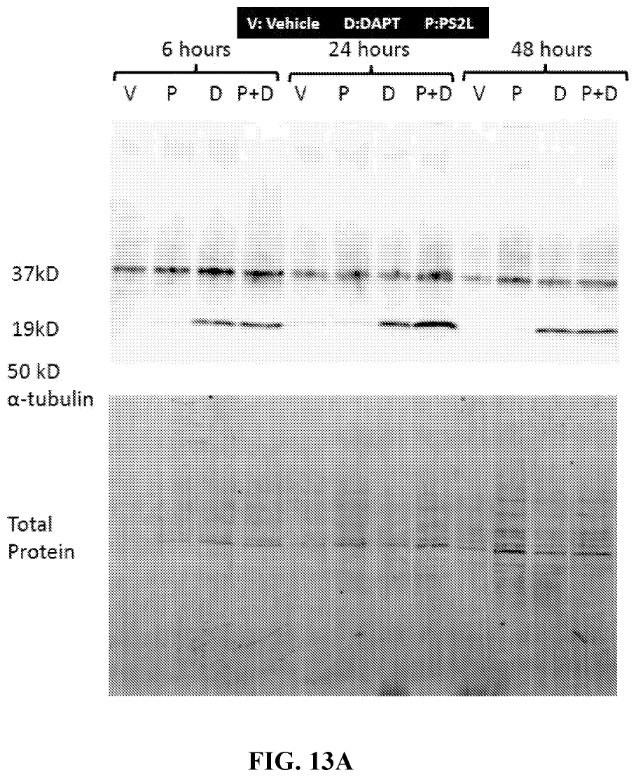

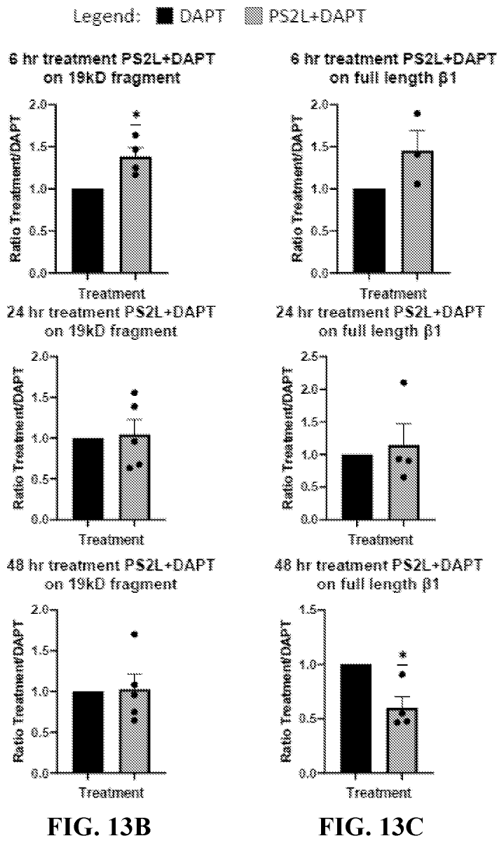

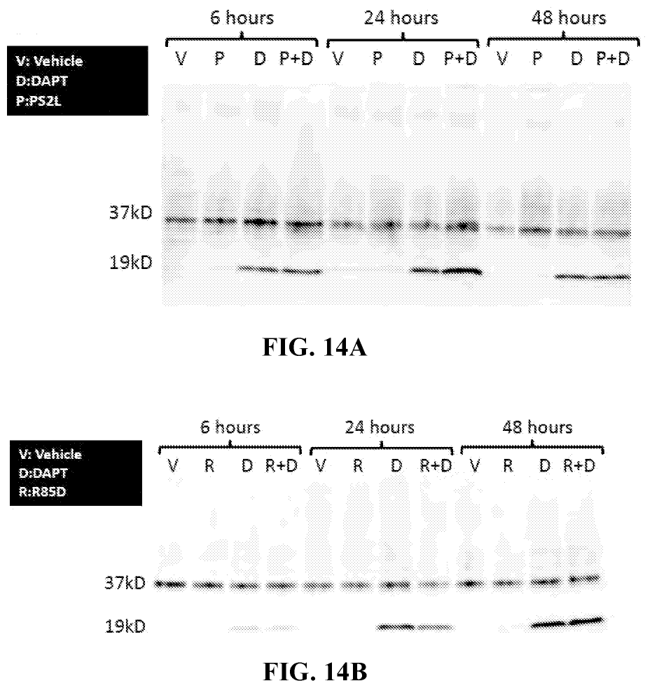

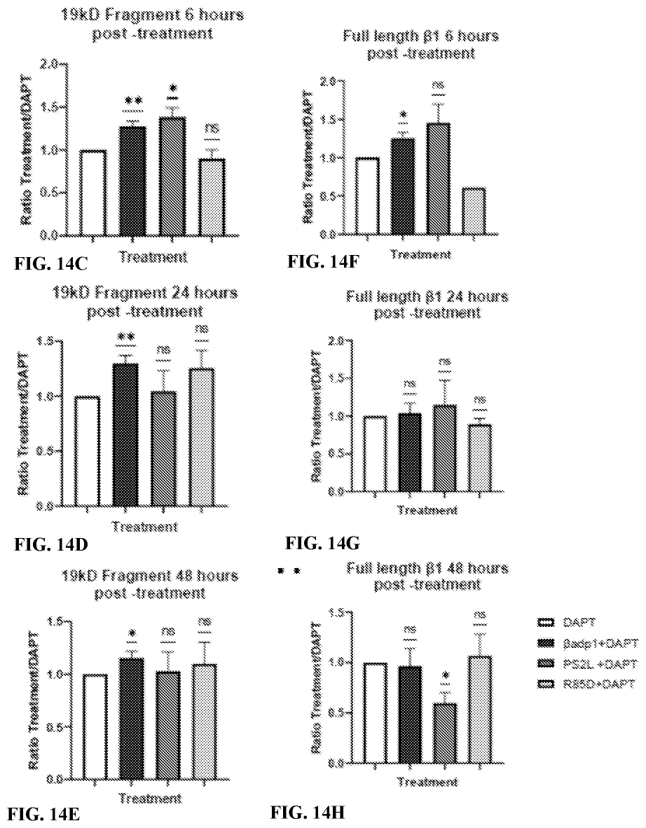

β1 has been reported to undergo a process of sequential intramembrane proteolysis (RIP) by BACE1 and γ-secretase, ultimately resulting in the production of a soluble intracellular domain (ICD) that is translocated to the nucleus, with correlated effects on gene transcription [2]. The RIP process is shown in FIG. 11. Applicant sought to determine if the β1 targeting peptides affect the RIP process in 1610β1 cells and whether any effect relates to changes in the distribution and abundance of β1 immunolabeling we observed in this model. First, to confirm that β1 underwent RIP in 1610β1 cells, Applicant treated cells with the y-secretase inhibitor DAPT. In the presence of DAPT, it was noted that the 19 kD Carboxyl-Terminal Fragment (CTF) accumulated in Western blots from cells sampled at 6, 24 and 48 hours following initiation of treatment, similar to results published by others in CHL cells and MDA-MB-231 breast cancer cells [2, 3] (FIG. 12A). Without DAPT treatment the 19 kD CTF was virtually non-detectable at any of the time points sampled (FIG. 12A). Next, Applicant co-treated cells with DAPT in the presence of βadp1 or βS2L. All treatments that included DAPT resulted in significantly increased levels of the CTF (FIG. 12A). However, compared to DAPT alone, βadp1+DAPT significantly increased CTF levels at 6, 24, and 48 hours post treatment (FIG. 12B). βS2L+DAPT significantly increased the CTF of β1 at 6 hours post treatment compared to DAPT alone, though not at 24 and 48 hours. Neither βadp1 or βS2L alone appeared to have consistent effects on CTF levels on immunoblots, as seen in FIGS. 12A-12C and 13A-13C. The abundance of the full length 1 (37 kD band) on blots also appeared to show no significant variation by treatment or by time point (FIGS. 12C and 13C). A scrambled control peptide, Scrl (FIG. 1) showed no effect on β1 c-terminal fragment or full length abundance (FIG. 14A-14H).

βAdp1+DAPT Decreases Resistance in ECIS Assay to Greater Degree than βAdp1 Alone

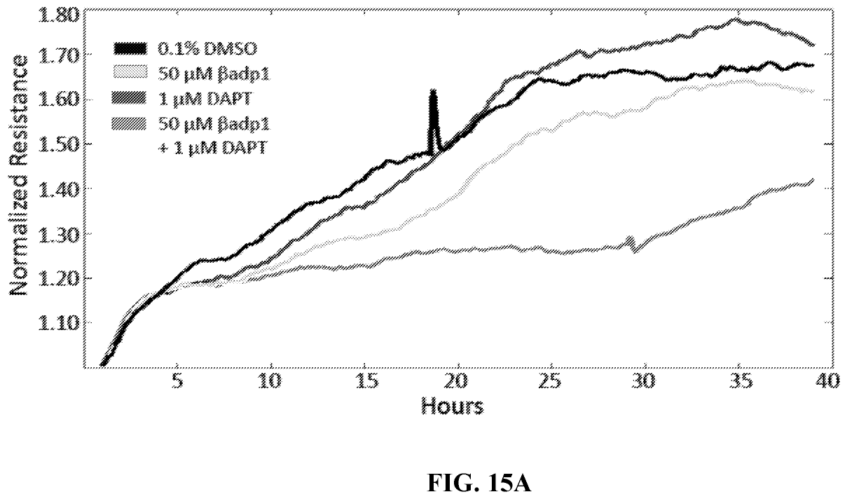

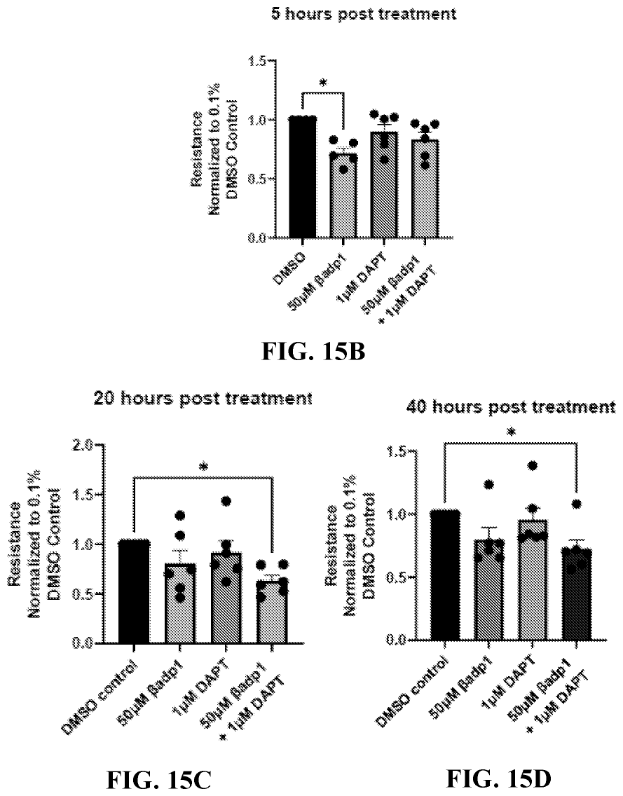

Applicant wanted to determine if inhibiting the RIP of β1 would indeed disrupt the later effects of βadp1 on the 1610β1 cells, resulting in increases in adhesion. Applicant repeated our previous ECIS experiments with βadp1 treatment over 48 hours, and included a treatment composed of 50 μM βadp1+1 μM DAPT (FIG. 15A). Applicant found that the cotreatment of βadp1 and DAPT resulted in disruption of the later effects of βadp1, with resistance decreased across the entire length of the experiment, with no increase in adhesion occurring after the 30 hour mark where we see an increase with βadp1 alone (FIG. 15B-15D).

### References for Example 2

1. Veeraraghavan, R., et al., The adhesion function of the sodium channel beta subunit (81) contributes to cardiac action potential propagation. eLife, 2018. 7: p. e37610.
2. Bouza, A. A., et al., Sodium channel 81 subunits participate in regulated intramembrane proteolysis-excitation coupling. JCI Insight, 2021. 6 (3).
3. Haworth, A. S., et al., Subcellular dynamics and functional activity of the cleaved intracellular domain of the Na. channel β1 subunit. Journal of Biological Chemistry, 2022: p. 102174.

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

Further attributes, features, and embodiments of the present invention can be understood by reference to the following numbered aspects of the disclosed invention. Reference to disclosure in any of the preceding aspects is applicable to any preceding numbered aspect and to any combination of any number of preceding aspects, as recognized by appropriate antecedent disclosure in any combination of preceding aspects that can be made. The following numbered aspects are provided:
1. An engineered polypeptide comprising or consisting of one or more βadp1 mimetic peptides or one or more functional domains thereof, one or more derivatives thereof, and/or one or more homologues thereof.
2. The engineered polypeptide of aspect 1, wherein the one or more βadp1 mimetic peptides or one or more functional domains thereof are any one of SEQ ID NO: 2-10, 38, 40, or 42.
3. The engineered polypeptide of any one of aspects 1-2, wherein the one or more βadp1 mimetic peptides or one or more functional domains thereof do not comprise or consist of a monomeric peptide having a sequence identical to SEQ ID NO: 2.
4. The engineered polypeptide any one of aspects 1-3, wherein the engineered polypeptide comprises a homodimer dimer comprising two monomers of any one of SEQ ID NO.: 2, 4, 7, 8, 9, 10, 38, 40, or 42.
5. A polynucleotide encoding any one or more of the engineered polypeptides of any one of aspects 1-4.
6. The polynucleotide of aspect 5, wherein the polynucleotide is DNA or RNA.
7. A vector or vector system comprising one or more of the polynucleotides of any one of aspects 5-6.
8. A delivery vehicle comprising one or more engineered polypeptides of any one of aspects 1-4, the polynucleotide of any one of aspects 5-6, the vector or vector system of aspect 7, or any combination thereof.
9. The delivery vehicle of aspect 8, wherein the delivery vehicle is a liposome, a micelle, or an exosome.
10. A cell or cell population comprising and/or capable of producing one or more engineered polypeptides of any one of aspects 1-4, the polynucleotide of any one of aspects 5-6, the vector or vector system of aspect 7, a delivery vehicle of any one of aspects 8-9, or any combination thereof.
11. A pharmaceutical formulation comprising one or more engineered polypeptides of any one of aspects 1-4, the polynucleotide of any one of aspects 5-6, the vector or vector system of aspects 7, the delivery vehicle of any one of aspects 8-9, the cell or cell population of aspect 10, or any combination thereof; and a pharmaceutically acceptable carrier.
12. A method of treating a SCN1B disease, condition, or disorder in a subject in need thereof, the method comprising: administering, to the subject in need thereof, one or more engineered polypeptides of any one of aspects 1-4, the polynucleotide of any one of aspects 5-6, the vector or vector system of aspect 7, the delivery vehicle of any one of aspects 8-9, the cell or cell population of aspect 10, a pharmaceutical formulation of aspect 11, or any combination thereof.
13. The method of aspect 12, wherein administering occurs for a time period of up to 2 hours.
14. The method of aspect 12, wherein administering occurs for a time period of 2 hours or more, optionally for 2, 4, 6, 8, 12, 24, 48, or more hours.
15. The method of any one of aspects 12-14, wherein the SCN1B mediated disease, condition, or disorder is a cardiac arrythmia and/or other cardiac contractility/fibrillation disorder and/or dysfunction, an epilepsy, a neurodegenerative disease, a neuropathy, pain, cancer, an Autism spectrum disorder, a mood disorder, or any combination thereof.

1. An engineered polypeptide comprising or consisting of one or more βadp1 mimetic peptides or one or more functional domains thereof, one or more derivatives thereof, and/or one or more homologues thereof.

2. The engineered polypeptide of claim 1, wherein the one or more βadp1 mimetic peptides or one or more functional domains thereof are any one of SEQ ID NO: 2-10, 38, 40, or 42.

3. The engineered polypeptide of claim 1, wherein the one or more βadp1 mimetic peptides or one or more functional domains thereof do not comprise or consist of a monomeric peptide having a sequence identical to SEQ ID NO: 2.

4. The engineered polypeptide of claim 1, wherein the engineered polypeptide comprises a homodimer dimer comprising two monomers of any one of SEQ ID NO.: 2, 4, 7, 8, 9, 10, 38, 40, or 42.

5. A polynucleotide encoding any one or more of the engineered polypeptides of claim 1.

6. The polynucleotide of claim 5, wherein the polynucleotide is DNA or RNA.

7. A vector or vector system comprising the polynucleotide of claim 1.

8. A delivery vehicle comprising one or more engineered polypeptides of claim 1, a polynucleotide encoding the one or more engineered polypeptides, a vector or vector system comprising the polynucleotide, or any combination thereof.

9. The delivery vehicle of claim 8, wherein the delivery vehicle is a liposome, a micelle, or an exosome.

10. A cell or cell population comprising and/or capable of producing one or more engineered polypeptides of claim 1: a polynucleotide encoding the one or more engineered polypeptides; a vector or vector system comprising the polynucleotide; a delivery vehicle comprising the one or more engineered polypeptides, the polynucleotide, the vector or vector system; or any combination thereof; or any combination thereof.

11. A pharmaceutical formulation comprising one or more engineered polypeptides of claim 1; a polynucleotide encoding the one or more engineered polypeptides; a vector or vector system comprising the polynucleotide; a delivery vehicle comprising the one or more engineered polypeptides, the polynucleotide, the vector or vector system, or any combination thereof; a cell or cell population comprising the one or more engineered polypeptides, the polynucleotide, the vector or vector system, the delivery vehicle, or any combination thereof; or any combination thereof; and
a pharmaceutically acceptable carrier.

12. A method of treating a SCN1B disease, condition, or disorder in a subject in need thereof, the method comprising:
administering, to the subject in need thereof, one or more engineered polypeptides of claim 1; a polynucleotide encoding the one or more engineered polypeptides; a vector or vector system comprising the polynucleotide; a delivery vehicle comprising the one or more engineered polypeptides, the polynucleotide, the vector or vector system, or any combination thereof; a cell or cell population comprising the one or more engineered polypeptides, the polynucleotide, the vector or vector system, the delivery vehicle, or any combination thereof; a pharmaceutical formulation comprising a polynucleotide encoding the one or more engineered polypeptides; a vector or vector system comprising the polynucleotide; a delivery vehicle comprising the one or more engineered polypeptides, the polynucleotide, the vector or vector system, or any combination thereof; a cell or cell population comprising the one or more engineered polypeptides, the polynucleotide, the vector or vector system, the delivery vehicle, or any combination thereof, and a pharmaceutically acceptable carrier; or any combination thereof.

13. The method of claim 12, wherein administering occurs for a time period of up to 2 hours.

14. The method of claim 12, wherein administering occurs for a time period of 2 hours or more, optionally for 2, 4, 6, 8, 12, 24, 48, or more hours.

15. The method of claim 12, wherein the SCN1B mediated disease, condition, or disorder is a cardiac arrythmia and/or other cardiac contractility/fibrillation disorder and/or dysfunction, an epilepsy, a neurodegenerative disease, a neuropathy, pain, cancer, an Autism spectrum disorder, a mood disorder, or any combination thereof.
